SEQUENCE LISTING (113 sequences)

SEQ ID NO: 1 - DNA, 1248 bp, Bactericera cockerelli
atgcagtcttctgcgcctctggtgctcatagcgttgcttacgaccctggtcactctggctagcaaggaggtacaaagcaaaatcaagcccggccgtgggagaggcagtaattggtggggaatagcgaaggccggcgaacccaacaacctcctcccaatgtcccccggtatggcgtacatggaccccaccgtacattcgacactcagacgaaagcagcggcggctggtgcgagagaaccccggggtctttctggccgtctccaaaggagccaatttagccatcaacgaatgccaattccagttcaggaatcgaaggtggaactgttcgacgcggaatttcttgcgcgggaaaaatctcttcggaaaaattgtagatagaggatgcagggaaacggctttcatctacgccatcaccagcgcggccgtcacgcacacggtggcacggtcgtgcgccgaaggcagcatagaatcgtgcacgtgcgactacagtcaccagtccaaaggtcaacggccgaagaccacgctcaacaatgtcgcgggcgtgcgagattgggagtgggggggctgttcggataacatcggatttggattcaagttttcgcgcgagttcgtagacacgggcgaacgaggtcgaagtctaagggagaagatgaacctgcacaacaatgaagctgggcgagcgcatgtcatggcagagatgcgacaagagtgcaagtgtcacggcatgtcgggctcgtgcacggtgaagacctgctggatgaggcttcccaacttccgagtcattggagacgcgctcaaagatcgctttgacggcgcgtcgagagtgatggtgagcaatgaaggaaacgtgcgaggcaccatcggcggcaagaagagtcggtacaacttccagctgaatccatacaacccggagcacaagccccccggggtgaaggaccttgtgtacctggagacttcgccagggttctgtgagaagaaccccgcgttgggcatccagggaacccacggacggcagtgcaacgacaccagcatcggcgtagacggctgtgacctcatgtgttgtgggcggggctatagaacgcaggaaatcaccgtggtggaacggtgcgcttgcgcgtttatctggtgctgtgaggtcaaatgtaaaacatgtagaacaaagaaaaccattcacacttgcttgtaaataatacgagtgagaagtattccggcctgggactagtcatccatgactttgtactaagctatg

SEQ ID NO: 2 - DNA, 1283 bp, Bactericera cockerelli
atgcctcacgtgagcagcgtctcaggccctggtgacgacctggggagcacagatgaggtgaaggtgttcaaggatgagggggaggacgagaagagatcctcggagaatttgaccgaggacaagaccagcttgatagatctgacggaatcacaggaaaaaagtgtctcccctaattacacctcgaataaaaatgcatcgaggtcagatctcagccctgttttcggcggcaaacttgaacccacccctcatacctctagttttaatatgggctaccttgtgtcgccctacccctatcctaacggcacgaccggacccctgcctgtctctatggcgagcaagatgggtctgggagccgcccacccgttcttcgtccacaacggtgaccccctggggcagcccccacccgcccacatgggcatccccccctaccacttggacaaggccgccggtgagtactggcggaccccacccatccaagatattaagcgactactagctgcctcaggactatcaaggccgagcatgtacccattctcagccggtcagtatccgtacccaatcctgagtcctgatatgagccaggttgcagcgtcctggcactcccctagcatgtaccccacgatctcatcaggtggctccggattccggggtgcctacccatcctcattgcctattacaagtacaagtttacctagtgatttctatagattctcgcccaccagcttgatggcggcacaccccggactgagcccccactcccacgccgccattgttacccccgggcccaaacaagaactcctctcagaccacaaccataggtcacatcataatgaacataaaaattcttcctcttcaaataacaatagtgataatacgaaatctcaagagtcagcccatagtaataataatagtaatagtaatgataaaaagaaacctcatataaagaaaccattaaatgcgtttatgttgtatatgaaagaaatgagggcaaaagttgtagctgaatgcacactgaaagagagtgcggccattaaccaaattctaggaagacggtggcacgccctgggcagggacgaacaggccaagtattatgagttagcccggcgggagcgtcagctccatatgcagctctaccccgactggtcctcccgcgcgaaccaaacccgcggcaagaagaggaaacgaaataacaaacaagagctgagcgattcaggcaatagtatgaagaagtgccgagcccggtacggactggaccagcagagtcaatggtgcaaaccttgcag

SEQ ID NO: 3 - DNA, 2322 bp, Bactericera cockerelli
ctacaagtccgtgtcgtaccaggcggccacctggtcgttgttctgtggcggctgaggcagggagttgccgagggggttgaagtctagctcgcctccgtagttcgagtccagatccagaccttccagagaggggagggtgtcgtagccttgctgatagttacgtggggccggggcgtagtcactgtacccctggtccggctccaacatgtcctgtagatcggggcccatgcccaggtcgcccccgttccagttcatgtgatcatccgagcggaacagagagttggtgagctccagactgagccgcttcttgtagtcattgggcttatcctcggacatgcggaacagaactgcggccgcgtaggtggcaactccctcgttgcgagagtgcagcagatcggtgagcggagcggtggctccctcagcctctatggcctcggctccttccttgtcctgtgcgagctcacacaacaccccagcagcgacgcgctgaatgttctctatctcgttgaacagcagagatacggccagacgaatcacgtgctgggcgcggatcagggcccggttgttcgactcccgggccagtatgtgcagagcccccaccgtgccctccactatctcctccatgcgcacaccatccgggtaggtgcccccgccgccgcccacactgccgccgcccggtgccgaggcggagctcgccacggaacgccgttgcgtgtccgtgaaggcgcggttcagcaggatgacgagcagatgaatggccccgtactcgcggagcgggctgtggttggcctgacacagggccaggttgcggatcaacccgatcacggctttgacgaggggccagcgcgagggagggttgagcaggttgacgatggtctggatgccgtagtggaggcgcacggcgttctgcgccatctccgattcgacgtggcgcgaggtgaggtggcggagggcgcagaccgcgggctccgtgatctcctctctgtccccggcgttcacaatggtttgcaccaaggcctccactccacccacttgacacaccgtcaccttgttacgctggttgttgcaggtcaggttggagaggactccagccgcgcaggtgatgacgttgatgtcttgcgaggccagcagctgcaccagagactgcaggagagactccagaccatccaccttggtgccagcatcggacaggttacgcagggtccagagacagttctgcaccaggcgttggctcgggtggcccaggtgcatggctagggcttgcatgccaccagcctccacaatggctggcttgttggatgagcacacactcaacactttgagcacccgactggtgcaccagagcagcttctcgtagtcgtaactgcgcatgatccgcaccagttccaccgggccctgcgaggcgagaatgatgagcttgctctcctggttcccgtaggcgaggatctggaggcagtcggtcacgatggccaggaacttgacgttgtttcggccgaggaggaggaccatcttctggaggccgcccgcgaggcggacagccatcttggagccctcctggtgcaggagtaggttgtgcagggtggtgatggcgtagaagaggacggactccaccggggagctcagaagtttcaccagggccggaatgcctccgctcttgaagatggccagcagtccctgtcggtgatgggacaagttgtgcagcgtgcccacggctcccttggtggtctccagatcgttcgagttggatatggcgtgcaccaaagctgccaccatctgagggctgttcatgatagcgtgtctagaagcctccttcttggagagctgatgaaccatcatggcggcttgtgagaccaccacctggtcctcgtcattgaggagtttgatgagctccgggatagcacgagtagccaggtcagcatcatcctggtagttgatcaggttcaccacagcatgcttcagcatttgactgggctccgtcaagcgctgcacagcagtgggagctgccgtatcaaattgagttgatggaatctcaatgccttcttccagtgtctcgggaaacatagcagcccgaacccgctgactccgagtctggctcagctgagaattcatttcatccacttgatcctgggtgaacccctgtccaaagccttggtccatctcaaacatgagctgatctccgtccatgtcctcctcctttcccgtgagtgacggagcattggtgttgactccagagtggattccagagtccaccagatagttctgttgctgccacatattgaccgagtggtcctttaaatttgtttgttgtcgagcaaagctcat

SEQ ID NO: 4 - DNA, 801 bp, Bactericera cockerelli
  misc_feature (147)..(147): n is a, c, g, or t
ttacgtaaatgtgtgacctggcgagcctacgcatacaagtgggtaggcatatagctcgagccttagcttacgcaagcaattgttagacaattcatgggatggctgggtccggggtgggctgggtcggatatatattacgaagcccancgcttccaattccttgatctcgtcctccacagagcggtccttggtgctggctttctcctttggtctctccttgggcgccgtggtggggatctctggcaactctcccccgggagtactcagcttcaggagatccttgtccagctcttcctgttccagggcctccagctccttctccaactcgtcctcatccacatcttgtccaaaggccacagggttggatatggcttctgatatctccttggctacgtcttgctgttccgcaatgtcatccatcatatcatgcacctggttcacatccatgtgtttatgtgctgccttgagtgcatcagctgcattcttcatggtggtaagtaccgcagtgtttgtgttagcaccctccagagcttcccgttgcatctcaatagttgacagggtgccatcaatttgttgcaattgtttctcataccttttctttcttttcaatgcttgaattgcagctcttttgtttttggttccattggttctagctatgttgatttcctgttctattttcttctccagaaactcctgtttctttataagcatgtcttctgtttctctcagtttttggatggcctctcctgtggtggggcccttttcctcagttttcccgaagatgtttttgaagaaactcat

SEQ ID NO: 5 - DNA, 1353 bp, Bactericera cockerelli
ttagccttcttgtccgaagtcctccatgaacttcctcagcttgaccatgtcatcatcattcactgtcggcttggaggtctccagagatttcaagatgtggttcatggacacagggggttccaaaagtttgtcggacggcacatccatgaagttcatttccctggcgcccggggtggagggggagcaaggggtcaacaggtcgttcatcgtcttgctcgggtccaccggtgaggggccgctggtgcggacgaaatgggtggcggactgccccagccgcaccggttgcatgagggcgtcccgcaccacaatgctgatgtcggctccggagtagccttcggtcctcttggccagttccttgaggttctcatcgctgatggtgttgcgggtgttgcccaggtggtgccggaacatgaagaggcgggcgttcagctcgggcagggcgatgtagattctcttctcgaagcggcggcggatggcggcgtccagcacccacgggatgttggtggctcccagcaccaggatgccgtcgttgtcgttgcccacaccttgcatttgaaccaagaactcggttttgattcgtctggcgctctcactttcgttatctgatcgggacgagcatagggagtcgacctcgtcaataaagatgatggagggcttgtgctgacgcgccagctcgaacagagtcttgacgagtttctcggactcgcccagccacttgctgacgaggtcggaggaggagacggagaagaaggttgagttgttggcctcggtggctactgccttggcgaggtaagatttacctgtaccaggcgggccaaagagtaggattcctttccagggtactcgttttcctgtgaatagctgcgggaacttgatgggcaagatgacggcttccttcaaggcctccttggctccttcaagccccgcaatgtccgtccacttcacattgggcttttccatcacgatggctccctccaaattggcctgcatctttttcttgtccgggtcctctccttcatcttcatcactctctttcttgtcatcctttgttcttgattcaccgtctttaactggttgtttctttcctttcttcagatattctttcagcttctctgctctggccaagtactcatcacatttctctcgtattgatgcctttccctctgctgatgtaatttcatacttgagtgcatggagaaaatattgcacactgtgtgtgtacagttttaatgcttcctcataattcttgtttttatctgcctcagtagccttgatggcaatattgattcctttctgcaagttagaagaatcatgactttcaatttgtggtaatttttgaaatgtgtctgacat

SEQ ID NO: 6 - DNA, 672 bp, Bactericera cockerelli
atgggggggttcttctctaaagccaagaagtccagggtcactgaacatgataaggccgttctgcaagtgaaacaacagcgggataaactgaagcagtaccagcagaaaatagagaagacacttgaatctgagagacagctagccagaaaactactccatgagggcaagaaagaccgagccaagctattgttacgtaaaaagaagtttggagaacaacttctctccaaaacagatagtcaactagagaacttggagaccatcatcaatgatcttgaatttgccacagttgaaaaagaggttctcaaaggacttcagactggaaacgaggcactgaagaaagtgaatgagctcatcagcatagaagatgtagagaggattctcgatgagactcgggagagtattgagaagcaacgggagattgatgagatgctgcaaggtgttctaacaactgaggatgaggaggatgttgagaaggaatacgagaagatgatggcagactccttggtgcctcagccggaacccagagtccccatcgcggagccagaggagtttgtcagcttacctgaggttccctcagaagagcccagcccagaggaaagtgtcgcgacggaaaccaaggctgtggagaaagagaaagccaagacaaaggaaaaggtgccggtgcttgcttaa

SEQ ID NO: 7 - DNA, 1848 bp, Bactericera cockerelli
atgtcaacagcattaggaaaaatggtggatgaagatagagaaggtagatttgggtttgtgtatgcagtatcaggtcctgtggtaacagcagagaaaatgtcagggtctgctatgtacgagctggttcgagtaggttactttgaactggtgggtgaaatcatccggctggagggtgatatggccaccattcaagtgtatgaagaaacatcgggtgtaactgttggtgaccctgtgttgaggacaggcaaacccttatctgtagagcttggtcctggtatcctgggtagtatttttgatggtatccaacgtccactgaaggacatttgtgagttgtctcagagcatttacatcccaaaaggagttaacattcctgccttgaacagagatgttagctgggagttcaatccaatgaacttaaagattggtagtcacatgaccggtggagatcagtatggtattgtacatgagaatacacttgttaaacataaaatgatcatgccacccaaagcaaagggaactgtaacatacattgctccagctggtaattacaaggtagatgaagttgttattgaaactgaatttgatggagagaagagtaaatacactatggttcaagtatggcctgtacgtcaacctcgccctgtcaccgaaaaactccctgcaaattacccccttctaacaggtcaacgagtccttgattctctgtttccttgtgttcttggaggaacaactgccattcctggtgcctttggctgtggtaaaactgtcatctcacaagctttatctaagtattccaattctgatgtgattgtctatgtaggatgtggtgaacgaggtaatgaaatggcagaagtattgagagatttccctgaactttcaattgaggtggatggagtcacagaatccatcatgaaacgtaccaccttggtagccaacacatcaaacatgcctgtagctgctcgagaagcttctatttacactggtatcacactgtctgagtacttcagggacatgggttacaatgtgtctatgatggctgactccacgtcacgatgggctgaggctttgagagaaatctcaggacgtctagctgagatgcctgctgacagtgggtatcctgcttacctgggtgcaagacttgcctccttctatgagcgtgctggtagagttaagtgtttgggtaacccagacagagagggttctgttagtattgtgggtgctgtgtctccccctggtggtgatttctcagaccctgtcacttcagcaacattgggtattgttcaagtgttctggggtcttgacaaaaaacttgctcagaggaaacattttccatccatcaactggctcatttcctacagtaagtacatgagagccctggatgatttctatgaaaaaaaccaccctgagtttgtacccctcagaacaaaagtaaaagaaattcttcaagaagaagaagatttgtcagaaattgtgcaactggttggtaaggcatccctggctgaatctgataagatcactttggaagttgccaaactgctgaaagatgatttcctccaacaaaacagttactccccctatgacaggttctgtcccttctacaaaactgttggtatgttgcgtaatactattgctttctatgatatgtcccgtcatgcagttgagtctactgcccaatcagaaaacaagatcacatggtctgtgataagggacagtatgaacaacatcttgtatcaactttcgtccatgaaattcaaagacccagtcaaggatggtgaagctaaaataagagcagactttgatcaactctatgaagacattcagcaagcattccgtaacttagaagactaa

SEQ ID NO: 8 - DNA, 1584 bp, Bactericera cockerelli
atgtcgatatctaccaagcaggctctacgggaaaacgtcctggctgtcacgcgggattacatttcgcagccgagaatcacatacaaaactgtgtctggtgtcaatggacccctggtcatcttggatgaagtgaagttccctaagtatgctgaaattgtacagctccgtttgaacgatggatcttaccgtgctggtcaagttctggaagtcagtggctctaaagctgtggtccaggtatttgaaggtacctctggaattgatgcgaagaacactgtctgtgagttcacgggggatatcttgagaacaccagtgtctgaagacatgttggggcgtgtgttcaacggaagtggaaagcctattgacaaaggaccccctatcctagctgaagactaccttgacattgaaggtcaacccatcaacccctactcaagaacctacccacaggaaatgattcagactggtatctccgctattgatgtgatgaactccattgcccgaggacagaagattcccatcttctccgctgctggtctaccccacaatgaaatcgccgcccagatctgtcgacaagctggcctggtcaagatgccgggcaagtcagttctggatgactcggaagataacttcgccattgtgtttgctgccatgggagtcaacatggagactgcccggttcttcaaacaagacttcgaggaaaacgggtccatggagaacgtgtgtctcttcttgaacttggccaacgacccgaccatagagcggatcatcactccccgcctcgccctgaccaccgccgagttcctcgcctaccagtgcgagaagcatgtgctcgtcattctcaccgacatgtcctcctacgccgaggctctgcgtgaggtgtcagctgcccgggaggaagtccccgggagacgtgggttccccggttacatgtacaccaacttggctactatctacgagagggccggacgtgtggagggcaggaacgggtccatcactcagattcccatcctgaccatgcccaacgacgacatcacccatcccatccctgacttgaccggctacatcaccgagggccagatctacgtggacagacagctccacaacagacagatctacccccctatcaatgtgctgccctccctgtcccgtctcatgaagtctgccatcggcgaggacatgacacgcaaggatcactccgacgtttccaaccagttgtacgcctgctacgccataggcaaggacgtgcaagccatgaaggcggtggtgggagaggaagctctgacacccgacgatctgctgtatctggagttcctcaccaagttcgagaagaactttgtgagccaagggaactacgagaaccgcaccgtgtacgagagtctggacatcggctggcagctcctgcgtatcttccccaaggagatgctgaagagaatccctgccgccacgctggccgagttctacccgcgtgactccagacacactggcgccaagttaacacgcgcgctggtcacccctccacccccctaccattgtctagacaagtgggaaccttacagcccctctaccccccctaccattgtggtctag

SEQ ID NO: 9 - DNA, 732 bp, Bactericera cockerelli
atgtcaggtaaagatagacttgccatttttccgtcccgaggagctcagtccctcatgaagtcccgtctcaagggggctcagaagggacacagtttgcttaagaagaaagctgatgctctccagatgagattccgtatgatcctgagcaaaatcatcgagacaaaaaccctcatgggtgaagtcatgaaagaagctgccttctccttagcagaggcgaaattcacaacaggggatttcaatcaggtagtcctacaaaatgtaaccaaggcacaaatcaaaatacgcactaagaaagacaatgttgcgggtgttactcttccagtgtttgagagttaccaggatggtacggatacctacgagctagctggtcttgccagaggaggtcaacagctcgcaaagctgaagaaaaactatcagacagccatcaaactccttgttgagctagcctctctacaaacatcctttgtaaccctagatgatgttattaaaattaccaaccgcagagtgaacgctattgagcatgtcatcattcctcgtattgaaagaactctggcgtatattatttctgagctggatgaactagagagagaagagttttaccgtttgaagaagatccaggataagaagaaggtgatcaaggcagcatcagaagcattcagaaagtcccgtaaatatgatgaggagcaagcaatgaatatgcttgatgaaggtgatgaagacattctgttctaa

SEQ ID NO: 10 - DNA, 681 bp, Bactericera cockerelli
ttaatcatcaaatttacggttagggttacgaccaaatatagcaactctaatgtcagggatgatctgttgtgcaatcaattccagacgagcctccaaggtattgctgatcttgatttttcctctttgagccaagagctcaatacctcctgtggtattaacaggtaagaattgttcactgtcaagtttcaagttaacttccttaccagcaacatcttggtattgcttggccaccgtaggtaagacaccattgacaagttctttgtcagcttcccgggcacggatcagaacattgggctccaataattgcagcaggccttgaaccattaacttttcaatgagttgggtgtacttggggcggtccttggacacctcacccagtctgttcctggcctcttccaacacatttctgacatgatcttccctcaccttcaaagccttcagtcgggcctgattcagcatgttagaggactgaatcttcttctgcagctcaacttgcttttctttcctgtcatagtattccataatcttgagacgctggtgttggaccaaacgccctttttcgatgttgaattcttcttctgccttagcatcgatttcctcagccttctcgttggcttcttgctcgatgaaagccatcatgtgattgatttgcttttgaacgtccgcatcgcttaaagccat

SEQ ID NO: 11 - DNA, 1278 bp, Bactericera cockerelli
caactcttggaattggtttttaaactgtgatgaaacccaggccagaccccatttcaagtttgtcaacataaattttagcgcttttgtccattgttcttgggaattgaattgatatttgacggaatatgtgttccctgtggtctcgtcttcgattttccccttcgagtccatattgtagggcaagtggaactcagtgtcccccttgcctaactcttccttgaactgctggaggcaatccagaaacgcgaccatggccatgtcaaatttggtgtcccagaagaatttggcacccccgctagcaaacagggggagatttttgtgctcggcagtatcctcaatgtaagagtggttgccgtagggcactatgcggtaccgctggaaggtgaggttgagtttcctggccagggctgtgagtaagagggccgtctgtccccaggccgcgttaatttcgctccagtccacaggggcggagggtagagtacccaggcggaagttgttgatggtgccaaagtggccactgtgccagatatggaaggtggcgttgaacacgtttgtgtttttgaacttgttcagctgagagatcgagtactggagctgcaactctaaactcttctgctgctcatcagtttgagaaagctcgtgttggtacttgcagtattcactccaatacttttcttcatcttgttttattctgtccttttcttcttcagctgattttatctctttttcgatgccttgttcctcatttttcaacatggctaactctcctagcaatctttcctcttcacactttaaatcgttaaattcttttgttaagaactccaaatcttcctctgaattagattctgaatccagcgttttcaagaaatcactgtattcctggaagtcctgctttgtgttttttaactctttgtccatcatgtcaagcagcgtatcagtgcattcttcacaaagtggatgatcaataccactgtttgaagataggaaatcgaaaagattagcatacactttcatctgatgtcctttagaattgcttgaatcatctttgccaagtaagcaaaatccattggaaccatttgttgaatctactagtttaaaggcaggaacaaaattgtctagactacttgcttgagattctaaatctatatcttgactagagcatattggtaaagagagttcagctaaggtatgacagttgatgttgtttagagattggtccagtatcaatggctgaaggcatcgctgacagacaaagcttacacttatacgttctaccat

SEQ ID NO: 12 - DNA, 1376 bp, Bactericera cockerelli
tttgcaaggtactctcggtcgtgcggcaacagatgtttgtctgtgatgaggaaatcgtcggttttgcaagcgtaggacacgcccttctgtttattcttatgctgtcgagaacttcgccacaactgaagccactccacatcctcggtgatgtcctcgcaccagtgttcacccgggtccaccatcttgttgtgaaccaccagagcgacactttgcttggtcgcagggtccaggacgattttccagaagatgtttggaactggcaggacgttgtccaagtacgggacgcgttgcaagtggatccgggctcggggaggctgatagaggtatggcttttcaccatacttgaggataccgctcacacctgtgtagacggtgagtggtcgcccccgctggatggtaaggcgtctgacgtcgctctccatggtgccatagttcccgctgttcagtttgtcccactggggcgcggagttgccgtagaagaaagacgccgtctgctccgtacggtatacgaaatctccttttggagccagatgaccacggttgaaagtcagtttacccgaatgcgtaagattgtcgtaggcggtctgtgtcatctgttcgcggagagtttgctgtgcgtgttggtagagtggcgacggatcctccatatgagggaaagccctaggaatacggaagttattgctcatagaaccatacatttgctgatatcttattgccttctggatctggtgaatggtatacttggcatggatcaagcgcggattgctatcgtcggcgcacacatcatacacattggtccaggccgatttcgaaggcgtttgccagaagcccaccttgtacatggtaagccgtttgtaacacttgaggctcgttttcctcatgatgactctcggaagattaatgcaggagtactcgttgtcgtacacaggacgacccaagtacatgactcttcctttggtacatttcacaatgacgagttttggcgggggactatgtctggacggtaatttggcctggaaattgttcaaatagttgggattcccctcatcttcctctgaacagatgagcatcacctcttgcccctcattcagcgctatgttctctccgtgctgagggtacactattgtgccaagcttaggatccacgaagaacgggtgctgccagagaagtttgttgatgttgatgtaacactttaggtcagaagatgacgaggctgatccgtagtagccagagttctctgtgtacgttgagggttgcaatgacttcatgaatggatgcttcttgactgatttcggttttctgttgtatccatgggattgtgacacttggtaatagatgactcccagccatatcatcacgggggcaagattgaccat

SEQ ID NO: 13 - DNA, 1695 bp, Bactericera cockerelli
atggttcaatattgggtcgttttagcgacatatttggtctcttttcatttcaaagaagtgttttgccgaaccacatgtagcattaatttgaacacggaatatcgcgcgtcagaggagcctttgtttttgagtcctgacggtgcgagtatcgtctaccctaacaacagagccagcattgaagtccacggaggagacaaactcagagtttactgtggacacaagaacttcaaggagctgaaatctcactcggacgactccctcttggttcaatgcaacagtgggaaatccttcaagctcctctctactcatctgcacaatgcccagtctgtccaatattctcaatctgacaaggacgtaccttccgacttctcaactggatcctcagatctccgtcgaactccgcactccagcgcttctctagaaccaaaatctttcgcctgctcgtctattgttcgctctgcaatacggcctggcaagccatgtgggttgaggggtagagaggagcatcatcaagagttcagcataggattcccgataagcaaaggatttcttcccattatatcagtatgtttcaataccgaactgaaatcggttctctggtccaggcatatcgtgtcacctacagtggaatatcacatgagcaaagtaccgcgcattacattcattcaggatagaatcccttacgatggatctcacctcaacgttgataagctttacaatcgacatcaagaaatagaaacaatcgcagctattctcggttccaaggatctagctgagaaatatattgaacccaaggggaacggattctttgcgaggggacatttggcacccaagggggactttgtgtatgctggggaacagctggctaccttccactttatcaatgtggcaccccagtggcagtacttcaatggaggacagtgggagaagatcgagtctactctccgcaactacgtcatcaggaccagggtgcaagccagggtactcactcttagcctcggcataacgcagctacaggacgaggagggggaggacagggacatctacctctactataaccagaggacgacggacaaactgatcaaagttcccaaactctacgccaagattatcaactttggcaacagctcagcatcatccaagagtgtggtcatagtgggggtgaatcagccctatgagagtcatgtgaaaatgtcaaccaatgatatcatctgcccggacgtttgcgacaggtacgaatggatagcgggtgtctcccagcacaatgaccagcatcatagttccggctacatctactgctgcactccctcagacttcatctcctacctacagtcaggacaaggctacaagctcgactcagctgcagccccaggcaactctgactcacatgaaaaagcactagattataatgctaaagctgaatcacatggtattcgttttcgattcagtgaaaatgttactttccaagatggttttggattgaatgagcctgctgattcgaataatcgttttggattaaatgaaacacccctgtcgtatggggatcgtgaatacaacaaaaatacggattctcttgcagatagttcaagtttggatgatttcagtaacagtgctctgcaggtcactgaggaagctgccgacacgctcataggacacctgagtgactttttaggtgctaccttctcatatagataa

SEQ ID NO: 14 - DNA, 1263 bp, Bactericera cockerelli
  misc_feature (1041)..(1042): n is a, c, g, or t
ttactgaaacttcagaatgcccttgatatccggcacaggtacaaatgaaacattggtgctcatctcacgaggatcacaacagtagcaaaatcctttcttgtagtcgtcttgcagttctgaccagttgttgctcgcacaaatgtcagttttacaaagcaactcaggagggttctccaggaaagggtcgttgctacacaccagggctatagccaggctggattgaggctcgtatatgattttgaagaactccgttggaactggtatgcccttgtttctaggatccaagtacacgggtgtgtttgcaagggtcagtattccgtgagtcccggtggttacgtccagagttgtgccaatattctccgccagctgtcgcacagccctctccaccttgagccagttgccggcgttgatgctctggaactgggggctcacgttggcgtaatagtaggtggcccactgccaggccgccatcaggaagtccccatcaggagccaggtggcctcgggacatgaacatcttgctgttgatgtacttctgggcctcggcctttgaaccgaggatattttcgaaaacgcgaacctgttgtgccaagttataagccttgttcggtgggagccctttgaacattgatttgtctcccactcggaagttgggtctcttgctctggctctgagcaccctgtatgctggggaagacctggtgtcgggtgtacagggtgcgggtctccttcacgttgtggcacaacgagaacaaagtagccaaagtatcgccctccacctggaagccaatattgaagatcaatgcattttccgaaccaccacattttgtttttgtggctttgatgtatgaatcagggtttttcctacacaccaaattatcttcagataagacatcgccgtctgcgttcacatttccgttatttccacattttcctgtgaccttgctctcctcgaactcctcgagtgaatttccttttccaggacacaagagaatgacctcctggtttttctgtaaagagagggccccatggtttttttttcnntggtttgtctgcaaagagagagccccatggttttttttcactgtgagttcaggaataatcaaattggagttactatcaacgaaaactggctgattatcaaacggaatgtcgtcctgcagatccagttggcattcttgtgaataagcagaattaaagaacactattgaaaagaaaataacggttgaacaacgaaaaaacaaacatctttgattggaatacat

SEQ ID NO: 15 - DNA, 1890 bp, Bactericera cockerelli
atgggtgccaaattgaaaggtggctttctgtttctcctggtggtgtgccaatgcagcggcgcctacatcaggagcgatgtaatcagcaacgatttgaacaggaacaatgttataagcaacgaattcaacagaaacgatatcagaaacgatgttatggttcgaggcaatgtggtcacggtgagaaaactaggacagagcctgacgagtgacctgcgactgttcgaagatttgccgccaatatgtgacagtctaatctactgccaaggagagttgctcaaggatgtctccttggcccgaatatacgaagactcaaagacttttgtggacaagaagctgaggttctcagagcgggagatcttgcagaagtatgcagcgctcaagggcaacgctggtaacagagccctcaccaaggagcagattcagaagttcgtggaggagaactttgaggacgcccaggagttggaggattggctgccgcccgacttcacggaccggcccgccctcatcagtctggtggcggactggaagtaccagaagtggctgcaactgctcaaccagatctggaagcagctgggcaagaagatgaacatcgatgtgctggtcaacgccgaccggcactcactcatctatgttgagaacggcttcttcattccaggaggacgctttctggagctgtactactgggacacctactggattgtgaggggggcgctgctgtgtgacatgcccagcactgctcgtggtatcatagagaacatcatatccattgtgaagcggtatggctacatgatgaatggcagtcgccgctactacataggacgctctcaacccccactactcatccaaatggccgccacttactacaccactactaacgatttgggattcatcaagaccaacatagggacgctggagcaggagttccagtggtggctgaagaaccgtctggtggcggtgaacaaggatggcaatgtgtacattctggcccactaccagacgaggacagcggggccccgacccgagtcctacagggaggactatgaaatagccgtcaacctgacttcggagtcacagaagaatcaggtgtacaacaatctcaaggcgggggcagagtctggctgggacttcagcagtcgcttcttcaatgataaggatggcaaagatacaggtggtttggagaacctggacacgccaaacttcatcgccgtagatctgaacgccatcctgcacgcgaatgctgtcactctggccgagtggttccacatcctggacgaccctgtcaagtctgcctactacaagaacctcgccaagcggttcctcatcggaattgaagcggttctctggtcggaggaggagggcatctggtttgactacgacctgcgcaatgaacaactgcgcaagaggttctaccccagcagtttcgcgcccctgtggaccggctcctacaccttccccaagtccctggtggctcggtccgccatacgccacttgatcaactacaatctcatcacgcccaactatacgccggtgtactacgggatgccctcctcgctacgcaacagcactcagcagtgggacttccccagctgttggccccccctccaagccatggtcattcagggcctggaccgcacctatgacatcaacgctcagctggtggcgtacaatctggcctccaagtgggtcttcaccaactacattgggtacagcaggacagggaccatgtttgagaagtacgacgccgtgaggctgggtgccccaggaggagggggggagtacatatcccagactggcttcggctggacgaatggcgtcattttcgagctgttcgcccggtgggggaacctgctcacctctgcgggcgacagcttccaaatcaaccacctctga

SEQ ID NO: 16 - DNA, 1842 bp, Bactericera cockerelli
atggcatctcctaactatgtggcccttctgggatgtttggccgcccttttactccaagtggtcacggctaaatacacacctctggattggtggcagacgtcaatcgtgtatcaggtgtatccgcgatcattcaaagatgtcaacggagacggtattggagacttgaaaggtatcatgcacaagataggctacttgaagaacctgggagttggcgccatctggatttcgcccatctacaagtctccgatggccgactttggctacgacatctcggactttcgggacatcgaacctgtgttcggcaccatcacagacttcaacaatctcctggccgagtgcaaagctaaaggcatcaaactcatcatggactttgtccccaaccacacgagtgacgaacaccagtggttcgtgaggtctgtggacaacattcagccctacagtgactactacatctggagaaacgccaagacggtcaacggtcaaagacagcctcccaacaactggctttcgaacttcggagggccagcatggacttggaatgagaagagacagcaatattactaccacgctttcgcccggcaacaacccgacctgaactatcgcaaccctcaggtggtcgaggaaatgaagaacatcatccgcttctggctagacaagggggtggatgggttccgcgtggatgcagtcccctacctgttcgagtctcctctcctggaggacgagccagtagctgaggacgcggcggagtacgaaccctcggaccacgactatctgaaccacacgctcaccaaggacctgccagagaccatcgacatgatctaccagttccgcgacctgttcgatgcgtacaaactgcaggacggatacaccagggtcttcctggcggaagcctacagccccctggacattctggtcaagttctacgggaacgccacgcgcaggggcgcccacttccccttcaactttctgcccatcaactatctgaaccgccaatccaacgccagagactttgcccgcgtcattctgaaatgggagaacagcaagcctgcctctgcttggagcaactgggtgatgggcaaccatgatcagaagcgtgtcgccagtcgcttcgacgcagagttcatcgacggcatcaacatgctcgccctcctcctgcctgggacggccatcacctacaatggtgacgagctaggcatggaggactccttcatccgctgggaccagtccgtggaccctcaggcactcaatgtgggaccgaagaggtacgagcagttcagtcgcgacttctgccggagccctttccagtgggacgccagccccaacgcaggcttcaccaccaacctctactcgtggctgcccgtcaacccgaactactggtacaagaacctcgcagtgcagcgtcgggataagcgctcccacttcaacgtgtacaaggctctggcgaggctgaggaagactgagaccatccagaggggcgagctcgatgtctatgacgtcaccgagtgggtgctctgcttcgtaaggagctacaaagaccaccccacctacattgtcgtcattaacatcggatctgaaatagaatacgcagacattctcactattcgtccttctctccctgagcagctgaacgtctacgccgccagcaagaactctgaccacgagcctgggaccactgtgaacacggacaagatccttctgaagcccaaagaagcacttaccttatccacggtggagttgtcactcgtccacaaacatgtggctagcggaccaaactacgaccctcacaacgccacattcctggcgatggccatgcctctagaagaagaggaagaagatttttaa

SEQ ID NO: 17 - DNA, 1878 bp, Bactericera cockerelli
atgccagcgattggaatcgatctgggaaccacctactcctgtgtggcggtgttccagcagggcaaggtggaaatcatcgccaatgaccagggcaacagaaccacccccagctatgtggctttcaccgacaccgaaagactgattggtgacgccgccaagaaccaggtagccatgaacccgagaaatacggtgttcgacgccaagcgactgatcggacgcaggttcgatgaccccaagatccaggcggacatgaagcactggcctttcaaggtggtggacgactgcaccaagcccaagctgcaggtggagttcaagggcgagaggaagacattcgcccccgaggagatcagctccatggtgctgacaaagatgaaggagacggcggaagcctacctcggacagagggtccgcgacgcggtcatcacagtccccgcctacttcaacgatgcccagcgtcaggcgactaaagacgcaggagccattgcaggtctgaacgtgctgcggattgtcaacgagcccacagccgctgcgctcgcctatggacttgacaagaacttgaaaggagaaagaaatgtgctcatcttcgacctgggcggtggcacatttgacgtttccatcttgagtattgacgaaggcgcgttgttcgaggtgcgcgccacagcaggggacacacatctgggcggggaggacttcgacaaccgtctggtgtcacatctcgcggaggagttcaagagaaagtacaagaaagacatgagtggcaacccccgggcgctgagacgcctgcgcacagcagctgagagagccaagcgcacgctttcctcaagcacggaggccagtctcgagattgacgcgctgtacgacggcgtggatttctacacaaagatctcccgcgcgcggttcgaagagttgtgcgcggatctattccgctccactcttcaacccgtggagaaagcgctgcaagacgcgaagatggacaagagtgctatccacgacgtcgtactggtgggcgggtccacgcgcatccccaaagtacaatccctgctgcagaccttcttctgtggcaagtcgctgaacctctccatcaaccctgacgaagcggtggcgtacggtgccgccgtgcaggctgccatcctaagcggcgaccagagtgccgctatacaagacgtgctactagtagacgtgacaccgctgtcgctgggattggaaacggcgggcggcgtcatgaccaaactcatcgagcgcaacacacgcatcccttgcaagcagacgcagacgttcacaacctactcggacaaccagccggccgtgacagtccaagtgtttgagggcgagcgcgccatgacgaaggacaacaacctgctgggcacgttcaacctgaccggcatcccacctgccccgcgcggggtgccccaaatcgacgtcacgtttgacttggacgcaaacggcatcctgaacgtgacggccaaggacaacagcagcggacgcagccagaacatcacgatcaagaacgacaaagggcgactgtcgaaagaggagattgataggatgattaacgatgcggagaggtacaaggacgaggatgagagacagaagcaacgaattgcggcaagaaacagcttggaagcttacgtgttcagcgtgaagcaggcgttggatgatgcgggcggcaagctaagcgagtctgagaagcgagcctgtcgggaggagtgcgacgcgacggtgaagtggctggacaacaacacgctggcggacagggaggagtaccaggagaagctgacgcagctgcagaagagctgcatgcctgtcatgcgcaagctgcatggcgcagccagtggaggcgacatgcccagtggtcccacggtggaggaggtggactga

SEQ ID NO: 18 - DNA, 1959 bp, Bactericera cockerelli
atgacaaaatttaccaagatgaagtggagtaaagttgaacaaaatgatgatgacggttccacggacccattgacacaggcgcaaatagccgaaatatatggagcgggctcccgtattcttagcacctaccacagatccaagcccaccgtaatgcagtctgccacctcctccacgtcttctgtggccactaatgcaggcagcacggcggcgctcatcaaactctccagcagcaagaaaagcaaaaccgtagctcaccagaagaaccgccagtacgcctatcagaagcacaacttgtacaacaagaacgccgccgccggcaaaatgatcgtggggacaaggaaggagatgaatctgctcctagcatcccccacagttcccgacatgctgtcggactctgaggacacttacaagcggtccagcgacagcgatgaaggcatccccactctcaattcaacagttattcgcatcaaagataggaaaaaggctgtcgtctacaagagagccattaaaaatcaagtattggcatcactcacagtttccctcggatcaatgatagtgggttttgcatccgcctacacctctcctgctcttccgtccatgaaccaaataggcagtcgagtttcccctaccgaggaagaggcctcatggataggtagtctcatgcctttgtccgctctcttcggtggaatggccggaggtccccttatagaatctcttggcaggagaactacaattctttctaccggtcttccgtttgttctttcatttctgttgattgccttggcaaatggcgtcccaatgatcttagctggacgctgtatagctggattctgtgttggcattgcatctctggctttacctgtatacttgggtgagaccgtgcaaccggaggttcgaggcactcttggactgctgcctacatttctaggaaatattggaattttagcatgctttgtcgctggaacttttctgaactggtacaagctggcgtttatgggagcatgtatccccattccgtttatcatctgtatgttcctcattccggagaccccgcggtggtacatcggcagaaacaaacagaagcaggctaggaaagcgctgcaatggctaagaggtgacgacgcagacatcagtagagagttcgcagagattgagaagatgaacaatgacggcaatgagggggatgacgagagttccacagggtgcagtgaggtgttcaaagcaatgtacatgaggccactcctcatcagcataggactcatgttcttccagcagatgagtggcatcaacgcagtcatcttctacacggtgaaaattttcaaggacgctggcagtaccattgacgagaactactgtaccatcatagtaggagttgtcaatcttctctccactctcattgccaccgcattcattgatagattgggtcgcaagatcctgctctacatatcatcgacggctatgtttattaccctggctactctaggaactttcttctattacaagagcttgggttatgacgtctcaacactgggctggttaccactgctcagttttgttatctatgtcattggattctcaatcggttttggctcgattccttggcttatgatgggcgaaattcttccagccaagatacgaggatcagccgcatccctagccacagcattcaactggacctgcaccttcgtggtcactaaaacattcaatgacatgacagcccttctcgggactcatggtgcattctggctgtttggtgtcatctgcctgataggtctatttttcgtgatcatcttcgttccagagacgcaaggcaaaagtttagaagacatagagaggaatctcaccggaggtggaggtggtagtagtagtgagggaggtccacgcgtgaaagtaccagtgagaaatgtcaggcggatgagttccattgctaacttgaagcccctacctataggagtatag

SEQ ID NO: 19 - DNA, 812 bp, Bactericera cockerelli
atgatccagcaaatagaatcatttcaaggtccacacgaaaaacgtcttcttgccaatctactcgacacttacaacactttggaaagaccggtagccaacgaatcggatccccttcaactgagctttggattgacgcttatgcaaattattgacgtggaatggaatgacgtgaatctaaggtggaactcgagcgagtacggcggaatcaaggacctcaggatacctccccacaggatatggaagccggatgtgcttatgtacaacagtgctgacgagggtttcgacagcacttatcccaccaatgtggtggtccggagcaatggtagctgcgtgtacattccaccgggcatcttcaagagcacgtgcaagatcgacatcacgtggttcccgttcgatgaccagcggtgcgaaatgaagttcggcagctggacgtacgatggattcaaggttgatcttcgccacatggatgagaagtcaggctccaacatagtggaggtgggcgtggacctgtccgagttctacatgtccgtggagtgggacattctcgaagtacctgccgtcagaaatgaaaagttctacacttgctgtgacgagccctacctggatatcacgttcaatatcaccatgcgccggaagacgctcttctacaccgtcaacatcatcatcccctgtatgggcatctccttcctcactgtgctcaccttctatctgccctccgatagtggagagaaggtgacgctgtccatctctattcttatcagtctccacgtgttcttcctcctagtggtggagattataccccccacctctct

SEQ ID NO: 20 - DNA, 867 bp, Bactericera cockerelli
  misc_feature (737)..(757): n is a, c, g, or t
atgccggagattaaaatcaagctcggactcaatgagctccaggacagcaagagcgggttgggcaaggctctcctcgcggagttgttcggcaacctcctgctcaactttttcgggtgtctgagttgtgtcagcctgctggagcagcccgccggcacgccgcccaatatcgtcctggtcgccttcacgttcggtctcgtcatcttcacgtccgtccaggctctgggtcacgtgagcggcggacacttcaaccccgccgtgacggtgggcatgctcgccacgggcaatgtgagcgtcatccgcggcgtgttctacgtggtggcgcagtgcctgggtgccatcgccggcagcctcatcctcaagtccctcacaccggtcgacttccagggcaacctgggcatgaccacgctcaacaagcacctgacccccacgcaggggatgggcattgagttcttcctgggcttcgtgctggtcctggtcatcttcggcgtctgcgacggcaacaaaccccacgccaaggcgcccgccgcgctggctatcggactgacagtcgctctgggacacttagccgccattgacttcacaggagccagcatgaaccccgctagaacttttggatccgcagtggtagcaaacatctggaccgaccactgggtgtactgggtggggccctctctgggcggtctcgtcgccagtctcctctacacattcttctttgtggcgcccaggatcgaggagtnnnnnnnnnnnnnnnnnnnnnacggcacgtttcatttgacaaagtttcgatttcatcattactgcctctcgaaaatttcatgtttttactctcgattttatgttcaaattttaatatcaactcgttgctaa

SEQ ID NO: 21 - DNA, 1269 bp, Bactericera cockerelli
atgtctgcctctgccttgtacatcctggatgtgaagggcaaggtcctcatctcccgcaactaccggggggatgtggagatgggggtcatagaaaagttcatgccactcctcatggagaaggaggaagagggcatgcttacccccctcctacagacgagtgactgcacattcgcctacatcaagtacaacaacttattcattgtatcaaccaccaagaagaatgccaatatagcgcttgtgtttgtcttcctcaacaagattgtgagagtattcacagaatatttcaaagaaatagaagaagaaagtattcgagacaactttgttgtaatctatgaactccttgatgagctgattgactttggctacccccagaccacagacagtaagatcctgcaggagtacatcacacaggaaggacacaagctggagatccagcccagaatccccatggcagtcaccaatgctgtgtcctggaggtccgagggcatcaagtacaggaagaacgaggtgttcctggatgtcattgagagcgtgaacctcctggccaactcgaatggcaatgtcctacgcagtgaaatagtaggtgctatcaagatgcgggtgtacctgtcaggtatgccagaactccggctgggactcaatgataaggttctgttcgagagtacaggacgaggcaagtccaagtctgtggagctggaggatgtcaagttccaccagtgtgtcagactctctcgctttgagaatgatcgcaccatctcgttcataccacccgatggcgagtttgagctcatgtcctatcgattgaatacccatgtcaaaccactgatctggattgaaagcgtgatcgagaggtttgtccactcgagagttgagtatatgatcaaagccaagtctcagttcaagagaagatccactgccaacaacgtggagattgtgatcccggtaccagcagatgcggactcccccaagttcaagacaacaataggcagtgtcaagtatacgccggaacaaagcgctatcacctggacaatcaagtcattccccggaggtaaggagtacctcatgagagctcacttcggcctccctagcgtggagaatgaggacgcagaggggaaacctcccatccaggtcaagttcgagattccctacttcaccacatcagggatacaggtgcggtacttgaagatcatagagaagagtggctaccaggcgctgccctgggtcagatacatcactcagaatggagactatcagttgaggaccacttga

SEQ ID NO: 22 - DNA, 4712 bp, Bactericera cockerelli
  misc_feature (4545)..(4548): n is a, c, g, or t
atgacacaaccattgcccatcaagttccaggaactactccagctcaccagtgtgggcatcagtctggggagcatcagcttcaacaccctcaccatggagtctgacaagttcatctgcgtgcgagagaagatcgcggattccgcgcaggtggtcatcatcgacatgaacgaccccaccacccccatccggagacccatcagtgccgactccgccatcatgaacccagccagcaaggtgatagctctcaagggcaaggctggcaacgacaataatcccaacgcgcccaagacgctgcagatcttcaacatcgagatgaagtcgaagatgaaggctcaccccatgaccgacgacgtggtcttctggaagtggatctcgcccaacaccctggccctcgtgacggagacctcggtgtaccactggagcatggagggcgactcccagccggagaaaatgttcgaccgccactccacgctgaacgggtgtcagatcatcaactaccgcacagacccaaagcagacctggctcctcctggtgggaatcagtgccgttcagaacagagtggtgggggcgatgcagctctactctgtggagcgcaagtgctcccagcctattgaaggccacgccgcctcgttcgccacgttcaagtccgaggggaacccggagcccgccactctgttctgcttcgcggtgagaacggcggcgggcggcaagctgcacatcatagaggtgggcaccccgcccgccggcaatacgccgctggcgaagaaggcggtggacgtgttctttccgcccgaagccgccactgacttcccggtggcgatgcaggtgagcagcaagtatgacgtcatctacctgatcactaagtacggctacatccacgtgtacgacatagagacggccacctgcatctacatgaaccgcattagcagcgatacgatcttcgtgacggcgccccacgaggcgacgggcggcattctgggcgtcaaccggaagggccaggtgctctcggtgagcgtggacgaggacaatatcatcccctacatcaacggcaccctacagaaccctgacctggccctgcggatagctgtgaggaacaatctggccggcgctgaggacctgttcgtacgcaagttcaatcttctcttcaccaacgggcagtactctgaggccgccaaggtggcagccaacgcgcccaagggcatcctgcgcaccccccagaccatagtcaagttccagcaggtgcccacccctgcgggccagaactcccccctcctccagtacttcggtatcctgctcgaccatggcaagctcaacaagcacgagagtctggagctgtgtcgcccagtgctggcccagggccggaagcatctgctggagaagtggctcaaggaggagaagctggagtgctcggaggagctgggggatctggtgaagcagtcggatccgaccctggctctgagtgtgtacctgcgagctaatgttcctaacaaggtgattcaaagcttcgccgagacgggccagttccagaagatcgtgctgtacgccaagaaggtgggctacacccccgactacgtgttcctgctgaggaatgtgatgcgggtcaatcccgaccagggggtgggctttgcgcagatgctggtgcaggatgacgagcccctggcagacataaaccagatcgtggacatcttcatggagcagaacatggtgcagcagtgcaccgccttcctgttggacgccctcaagaacaaccggcccggcgaggggccgctccagacccgcctcctcgagatgaacctcatctccgccccccaggtggccgatgccatactgggcaaccagatgttcacgcactatgaccgcgcccacattgccgccctgtgcgagaaggcggggcttctgcagcgcgccctggagcactacacagacctgtacgacatcaagcgggccgtggtgcacactcagctcctgaacgcggactggctggtgggctacttcggtaccctctccgtggaggactcgctggagtgtctcaaggccatgctcaccaacaacatccgcgccaacctgcagatctgcatccagatcgccaccaagtaccacgagcagttgaccaccaaggcgctgatagatctgttcgagtctttcaagagctacgagggtctcttctacttcctcgggtccatagtcaactacagccaggaccaggaggttcacttcaagtacatccagtcggcctgcaagacaggccagatcaaggaggtggagcgcatctgccgcgagtccaactgctacaacgcggagcgcgtcaagaacttcctcaaggaggcgaagctcaccgaccagctgccgctcatcatcgtgtgtgatcggttcgacttcgtccacgacctcgtcctctatctgtaccggaacaacctgcagaagtacatcgagatctacgtgcagaaggtgaatccctctcgtctaccagtggtggtggggggactactggatgtggactgtagcgaggacatcatcaagaatctgatcctggtggtgagaggacagttctccaccgatgagctggtagaagaggtggagaagcgcaaccgtctgaagctcctgctcccctggctggagtctcgcatacacgaggcctgcaccgagcccgccactcacaacgcgctcgccaagatctacatcgacagcaacaataatcccgagcggttcctcaaagagaaccagttctacgacagccgtgtggtggggaagtactgtgagaagcgggacccccacctagcctgcgtggcctacgagagaggagcctgcgaccgggaactcattcaggtgtgcaacgagaactccctgttcaagtccgaggcccgctacctggtgagacgcaaggaccccgagctctggctggaggtgctctccgaggctaacccgttcaagcgacagctcatcgatcaggtggtccagaccgccctctcagagactcaggacccagaggatatctccgtcacagtcaaggccttcatgacggcggatctgcccaatgaactcatcgagctgctggagaagatagtgctggacaacaatgtgttcagcgatcacaggaacctccagaacctcctgatcctgaccgccatcaaggcggaccagacgcgcgtgatggagtacatcacccgcctggacaactacgacgcccccgacatagccaacatcgccatcaacaaccagctgtacgaggaggcgttcgccatcttcaagaagttcgacgtgaacacgagcgccatccaggtgctgatcgagaatgtgggcaatttggaccgggcgtacgagtttgctgagagatgcaacgagccgggcgtgtggtctcagctggccaaggcccaactagcgcagggcctggtcaaggagtccatcgactctttcatcaaggcggacgacccgagcgcctacctagacgtggtggcgacggcgcaccagaccgagtcctgggaggacctggttcgatacctctcgatggcgcggaagaaggcgcgcgagagttatattgagagtgagctgatctatgcgtacgcgcgcaccaaccggctggcagacctggaggagttcatctcaggcccgaaccacgccgacatccagaagatcggcgaccgctgcttcgaggaccgcatgttcgagcccgccaagattctctacaacaacgtgtccaacttcgcccgcctcgctctcaccctcgtgcatctgagggagtaccagggcgccgtggactcggcgcgcaaggcgaacagcacacgcacctggaaggaggtgtgcttcgcgtgtgtggactccggggagttccgactggcgcagatgtgcggcctccacatagtggtgcacgcggacgagctggaggacctgatcaactactaccaggaccgcggccacttcgaggagctgatcaacctgctggaggccgcgctggggctggagagggcccacatggggatgttcaccgagctggccatcctctactccaagtacaagcccgccaagatgaaggagcacctcgagctgttctggtcccgcgtcaacattcccaaggtgctgagagcagcagagcaggcacacctctggtcagagctcgtgttcctgtatgataagtacgaggagtacgacaatgccgtcatcgccatgatgactcatccgactgaggcatggcgggagagtcatttcaaggatatcatcaccaaggttgccaacattgagctctactaccgcgccatccagttttacctggactacaagcctctgctcctgaacgacatgctgctcgttctcacaccccgtatggaccacacgcgctcggtagccttcttcgccaagagcgacaacctccagctcgtgaagccctacctgcgctccgtccagtgtctcaacaacaaggcgatcaacgaggcgctgaaccagctgctcatagacgaggaagactaccagggtctgcgcacaagcatcgacgcgttcgacaacttcgacacgatcgcgcttgcgcagaaactggagaagcacgagctgacnnnnaagaaggacagactgttcaaggacgcgatggagtacgcggccgagtccaagaacacagagctggccgaggagctgctcgcctggttcctggagcgtcgcaacttggactgcttcagcgccaccctgtaccagtgctacgacctgctgcggcctgacgtcatcct

SEQ ID NO: 23 - DNA, 4077 bp, Bactericera cockerelli
atggatggctttggtgataactttgatgacaatgatgttgatcccgcagcagaattcatagccagggaacagaatcaattagcaggcctggaagatgaagttcagccagctgtagccagtcttaatccagcaaacaatggtactctttccccgcctgacattaataatgactttgagtttgtatctaacactaaccctgaagacatggttcatattaatcattcagataatgttcttggaaatgaggaggctcaaagttctccagtggtttttaatgttgctgaccctgtggctcctgtactcaatggtcatagttcagctcatgatgaagaagccataccaccaccttcaaaagctactgacattagacctaaattgttcagtcagcccccaacacctgccaaaacaccaccaatacgagaggaacctgagaaaatcatcaaatggagagaagctcaaaagaaacgactagaagagaaagatgctgaagaagaaaagaaaaaggaagagatgagaaaagctgccaagcaagagctagaagaatggtatcaccatcatgcagaactcattgctaaaacgaaagctgccaataggaatgcagaaaagcagtttgtggcggaggcagacgacatagagcccgggacagagtgggagcgcatcgccaagctctgtgacttcaaccccaaggtggggagaaccaacaaagatgtgtcgcgcatgagatctataatcctacagctgaagcagactccgctgaggaaagactaagaccaatcctacagctgaaacagactccgctgaggaaagactaagaacaatcctacagctgaaacagactccgccgaggaaagactaagaacaatgccctcgaatttctgggtgtgtttccatgggaggaaaaactatacaatatctagggagctctgttcaaactatgagggtagagggacatcacaaatacctgaagtatgtatttacagctgaaacagactccgctgaggaaagactaagaacaatgccctcgaacttcttggtgtgtttccatgggagaaaaaactatacaatatccgagagtccctctagcaatatctagggagctctgtttaaactgtgagggtagagggacatcacaaatacctgaagtatgtatttacagctaaaacagactccttctatcatcttatataacaaatggaatgtgtttttttctttagttttcaagtttacatttttgaagctgtaggtattgttatgtatctccattattttaatatgtgcactcgccctcgtgagtttctcagtaatatataaataggtatgttaaatattcatcaatatcctaacgttagcgtgctactgagcgtatttccagtctttggaaagaaatcttgagacctaaatgtaataaactaaatggaaaaaaacctattaatctaacattggggtcgggttctacgaaaaacacctaacctaaaaaacacctgaaaaacacgaaagatgttacttccaaagctatcttgacgaatcctttggaactatcgtttttcaggtgtttttcgtagaacccgaccctattcaactgagtttaactcgaactttttttccaagttgtaataatgatattgtgtgacgaaaaattccacgggtacaccaccacctggagatgtttcaataaataagcgtctgtagctaccatttagctcttggctggatcttccacgagagtcggaatattgcatttctgtgaggttttgaattagttatgagttatgactacatgtgacagtgcgtcgaggtgtcggtgataactgcaagctaaatttcatatttgcaacacattctttctgtgggcattctatattacccatagatagctcattgaaaccgattcacccagagacttaaaccagtattctgttcataacatgtgtaaaccagtctagagtattgaaaactgaactttgtgtatcgcaaatgttctaacaagtcaaaaaaatatcctatcaagggctcttttccagtcatgattagttgattttaaaaataaatccaaacgaggtgttgtgtgaactattcgggactcggcagtcatttgatcgcctacattgaacactaaacttgtcataaatatttgtagagtatttaactccaaaattaaacattctaaatatttgtgggtatccaatatccatgttactgtatctatgtatttattgtaattcataaatcattattgggtgaggtgggtgatatagtcacgatgaacaaaaaaagtctagtagtttcaaataggaccggtactacaaagttatttttggcctttaccgagttactattattgtttgtttcatttagattagttcaaatttatcatctgatgtaattcaacttggttcaagtgaacgttattgtctttctagcattctaatgatttttgattacctcaaggaagtaaagcggtaggtagagaccacgtccctacaacgttgaatggtactgttcagaacgaagtaatgttttcgttttaattaacgatatttcccttcggatatacatgactaacaagtaaaagtcctcattctgaacatccgccatatttttgtgaagttaacaatatgttatgtaaaagcagtgcacatatgagaattttccaacagtatgctcacaaaactccacaagatcatgacatgagcttaaaatctcagtaagcctaacctctccttttcttgcttgccttgtcatgtaagaggacaattatttttttagtgataataataattcatttttgtacatgtccaagattataaaaatattacatacagagtgtagtaatgaacaattacttgaacttcatcttctataggtacaaaaagttggggttattctatttttagttattttaatgattaataagtaggtattctaattcagacaataataatcattatcacaccactatcagttaagtttaagatctatctaaagacaattacaagatctaaattattattattttactctttataaggacataatatatacaacgcagataattacaaaataatatacaacataagaagaagtaaaaagagaaataaattgtttttttttttttaatttatacatatgtcctaactaaagggcggggttattgcctttggcaatctcttccacccaaccctttaatctaaacctaaatgattcattagaggtgtgagaggtactgtgctttaaatttgtcgtaactttttgtatttttttaaataagtgtgaattattttgtttaaataggtattttttaattttcaaaatccaaaaagttctgatgataaaaatagaacttcccttacctccgcccttgaattattgtttaggatatgtattgtgattgcccttagattaaaaaaattgaagtcaattgatagtggtaaaattaatatcagactaaccactcagaaggtgaagtgaaaaaaactactcaatttttgcaaactcttcggtgggtcttcaacatattatatatttttgttgaattcaaggtcaaattgaaaggggtttatttcataataaacccccttttggttgacctgtagaatatttagttcaagtaattggacattattttgttacaccctgtagaaaattctgtgttatccagtttctaggtttaatattgcaacttatgttgtcataattataaatattatatgtttctgtttttgtactctttaagttccggccactactcaggggtaacatagaatacattccactaattaaaagtatgaaacatcatgaatgaacatgaaaattatgattctagttctgagaaagagttattcgaaatctaaactcatatacagagatgtacattttattaataatgaatgctacagttgctacatcttccactctacatgcgcttttacctggcaaaagtatcttaacagggcctttataatcgtcgagtcaggtggacgtaatctgaatggattccactttaatcacagattccaagaaaagttcaatgcaaactctgagtatggtggtcacatttataattgttatgttcaataa

SEQ ID NO: 24 - DNA, 1326 bp, Bactericera cockerelli
atggatgaagagtatgatgccattgttctagggactggactgaaagaatgcatcctcagtggaatgctgtcagtgtctggcaagaaggtgctacacattgacagaaataaatactatggtggagaatctgcttccattacaccacttgaagaattatttacaaagtttggttccactgtacctgatgaagtaacttttggtcgtggacgagactggaatgtagacctcattccaaaattcctcatggccaatggctctcttgtaaaactacttattcatactggtgtgacaagatatttggaattcaaatctgttgaaggaagctatgttttcaaaggtgggaaaatatccaaagtccctgttgatcaaaaagaagctttggcttctgatttaatgggactctttgaaaagagaagatttagaaatttcctggtatacattcaggagttcaatgaggcagaccccaagacatggaaagatatcaatcctcaaacttccaccactgcgcaactctatgaaaagtttggacttgatcccaatactcaagatttcacgggtcatgctctagctctctaccgaaatgatgaatatattagtgatcttgccattcatactataaaaagaatcaagctttacagtgactcactagcacgctatggcaagtctccatacttgtaccccatgtatggactgggagaactgcctcaaagttttgcccgtctaagtgcaatctatggtggaacctacatgttggataaacctgtagatgaaatagtgtttgagaatggtaaagtagttggtgtacgatctggcagtgaaacagctcgctgtaagcaagtgtattgtgatccatcctacgtacaagatagagtgaagaaattaggacaagtcatccgctgtatctgtcttatggatcatcctattcccaacacaaaggatgctctctcttgccaaattatcattccacagaagcaagtgaaccgcaagtctgacatctatgtctcacttgtgagttacacacatcaagtatctgccaagggttggttcattgctatggtatctaccacagtagaaactgacaatccagaggttgagatcaagcctggacttgatctcctcggctcttacaagaaaaaatttgtcattgtttctgattattttgaacccacagaccttggatcagaaagccaggtcttcatttcaagttcatatgatgctacaactcactttgagaccgtctgcacagatgtggtcaatttattcaaaagaggcacaggagaagattttgatttttccaagataaaattggaacttgaagaatcttga

SEQ ID NO: 25 - DNA, 354 bp, Bactericera cockerelli
atgtccggggacgcaggactacagccgggaggagcggggggggacgatggcatcctggggggaccgcggacgcctcaacagattgctgcgcagaagcggttgcagcaaacccaggctcaagtggacgaggtggtggacataatgaaaactaacgtggagaaagttctggaaagagatcaaaaactttctgaacttgacgacagagcggatgcacttcaacaaggagcttcacagtttgaacagcaggcgggaaaactgaaaaggaagttttggctgcaaaatttaaaaatgatgattattatgggtgttattggcctagtcatagttgccatcatagtgggtaagtttaactga

SEQ ID NO: 26 - DNA, 1331 bp, Bactericera cockerelli
atgaagatcatctccttcaacgcctccaccctgccccccctcgagtctgacagcatcaaacggaaaacccaggccactaccaccaccacacccctgcctgaagaggaagaagaagagttcgacacgacggaagtatacgaagatgtttcggccgagatcggcagcacggtggcctccatcaagaaggagattgccaacgagtcgatgaaggtggcggaaaagttcgccaaggagactgggatgcctacgtgggtggtggtgtccatatttttaggaatagctatactcattgtcggcatttgcggatgttgcgtctaccggtgctgtaaaaagcgtcgcgccaaggatggcaagaaaggcaaaggggtcgttgacctcaagtctgttcagctcctcggctcagcttacaaggaaaaggtgcaacccgacatggaggagctgactgagaatgcagaggacatagcagaggacggagacaagaaggaggagatcaagctggggaagctgcagtataagctggaatacgacttcaacgccaacagcttgtccgtgacggttctgcaggcagaggatctgccggccctggacatggggggcacctccgacccctatgtcaaagtctacctgcttccagacaagaagaagaagttcgagaccaaggttcatcgcaaaactttgaatcccgtctttaacgaaacgtttgttttcaaaggagttccatatgcagacgccatgaacaagactctggtgttcgccattttcgatttcgaccgattttccaagcacgatcaaatcggggaggtaaaagtggctctttgccagatcgatctggcccaaaccattgaagagtggcgggaactgcagagtgtagaaggagaaggaggacaggataacaagttgggagacatctgcttctctctgcgctacgtccccaccgctggaaaactcaccgtggtgattctggaggctaagaacctgaagaagatggacgtcggaggattatcagatccctacgtgaaaatcgccctcatgcagaatggaaaacgacttaagaaaaagaaaacgagcatcaagaagtgtacgctgaatccttactacaacgagtccttcacattcgaagtcccctttgaacagatacagaaagtgaacctccaagtgacggtggtggactacgatcgcattggcacctcggagcccattggcaaggtggtgctggggtacaacgccagtggcaccgagctgaggcattggtcagacatgctggcgtccccgcggggcctatcgcccagtggcacacgctgaaagacccggaggatgacaagaaagattaa

SEQ ID NO: 27 - DNA, 1526 bp, Bactericera cockerelli
  misc_feature (138)..(139): n is a, c, g, or t
  misc_feature (1341)..(1342): n is a, c, g, or t
tagatttactaatcctgttttagaatacaatcctaaattgtcattcagggaggagaggtagatgaatttcaagtcatgcagtaggtgttgatagagagtactggaatagtttcacttgatcctgattggctggcgagnnaggcgacgaggggagttgcagacgtctgtccagtgagacaggacggtgccagagctcttgtccccgcccagatcaagtcggccaatcacctcattcttggtgacgcggtcccaatccagcagcaacagttccagactcacgctgtccaagttgtccgcgggcacctcgaacacgaaggactcgttgtagacggggttgagggttcgcttcttgacgtgggtcttctttttggcgacccgctgtcccttgtacaggaggtacaccttcacgtacgggtctgctagtccggtgacgtccatcttgggtagattcctcgccttgaggatgacgatggtgaagcggttagcggccggctgccagcagagagacagcaggatctcaccccggccctgcgactttattcgcaggctgcggggctggatgtcgcgacagaaggagagggagtggccggtggtctcgaaggactgcagagagtagaagacctcgcccacaatgtcgtcccgggagtagcggtcgaagctgaacaccacgaagtggagggtggagcccttgagctggttgacggtgaccccggtgaaggtgaactcctcgttgtagacgggattgcgcgtcttgcgcaggacgcgcgtcttgaccttctgcttgtcgggcagcaggtgcaccttcacgtacgggtccgagctgcccgacgcgaggtccttgatgcagaggtcgcggcacttgatgacgcccaccaccagaaggttctccttcgcctggtactttatcttgaagaagatctggccgagtttgccctccgcgttgccgtcgcagtcgatgagggcggcctccttgctgccggccacggtcacgagggaggagtcgtcactctcgttgccgcccggggtgggcgtggtcgagccggcaatgtccatgaggagggagcggtccgggcacgctttttcgttctcggtggtgacaagggtgccgggcagagccacacagggctgcataggactgtggtggtgactgccgccgccaccggcggaatgggtggagccagtgggactgttgaccagcgacgccacgggcgtggcgtaggtgggcgagggcgatttcttgagatagtgcgagccgttgctgccggcaggcgagcgtacggcggtgggccgcttcaaggtcatctgagcgagagccaacttcttcgtctcggccgcggaggnnttagtttcggccgcggaggggcggcgcctacaatagtaggtggccagtcccacggtggcgaggaatacgaggccggccagacccaggcccagcactgtgtggcccgtcacgttgtcaagggactgaatgtccggaccctctccgttgtgcaccataatggccatcttgagaagtcctccaccag

SEQ ID NO: 28 - DNA, 1131 bp, Bactericera cockerelli
atgtgtgacgacgatgtagccgctttggtcgtggacaatggttccggtatgtgcaaggccggattcgccggtgatgacgcccccagagccgtgttcccctcaatcgtcggtagaccccgtcatcagggtgtcatggtgggtatgggtcaaaaagactcctacgtcggtgatgaggctcagtccaagagaggtatcctcaccctgaaataccccatcgagcacggtatcatcaccaactgggacgacatggagaagatctggcatcacaccttctacaacgagctgagagtcgcccccgaggagcaccccatcctgctgacggaggcacccctcaaccccaaagccaacagagagaagatgacccagatcatgtttgagacgttcaacacccccgccatgtacgtcgccatccaggctgtgctctccctgtacgcctccggtcgtaccaccggtatcgtgctcgactctggagatggtgtctcccacaccgtccccatctatgaaggttacgccctcccccacgccatcctccgtctggatctggctggtcgtgacttgaccgactacctgatgaagatcctcaccgagagaggttactccttcaccaccaccgctgagcgggaaatcgtccgtgacatcaaggagaagctctgctacgtcgccctggacttcgagcaggagatggccaccgccgccgcctccacctccctggagaagagctacgagctgcccgacggacaagtcatcaccatcggaaacgagagattccgttgtcccgaggctctgttccagccttccttcctgggtatggagtcttgcggtatccacgagaccgtgtacaactccatcatgaagtgcgatgtcgacatcagaaaggacctgtacgccaacactgtcctgtccggtggtaccaccatgtaccccggtatcgccgacagaatgcagaaggaaatcactgccctggctccttccaccatcaagatcaagatcatcgctccccccgagagaaagtactccgtatggatcggtggttccatcctggcctctctgtccaccttccagcagatgtggatctccaaacaggagtacgacgagtccggtcccggaatcgtccaccgcaaatgtttctaa

SEQ ID NO: 29 - DNA, 1803 bp, Bactericera cockerelli
atgtcctactcaaataagtacatttttgcaaccttacctagaactcagagaggtcaaccaattgtcttgggaggagaccctaagggaaagaactttctttacactaatggaaacagtgtcataatcaggaacatagagaatcctgccatttctgatgtctacacagaacattcttgtgccgtcaatgttgccaaatactctcctagtggcttctacatttcctcaggagatatttcaggaaaagttagaatctgggacactgtgaacaaggagcatattctaaagaatgaatttcatcctattggaggacctatcaaagatattgcttggtcacctgataaccagcgtatggttgttgtgggtgaaggaagagaaagatttggccatgtttttatggcagaaactggtacatcagtgggagaaatttctggccagtcaaagcccatcaactcttgtgatttcaaaccttctcgtcctttccgagtgatcactggtagtgaagataatacgattgctgtgtttgaggggccaccattcaaattcaaaatgaccaaacaggagcattccagatttgtccaagctgtgcggtattcgccatcaggtagtcattttgcctcagctggatttgatggaaaagttttcctatatgatggtggttctgcagatctagttgcagaacttggcagtcctgcacacaagggtggtgtttacggtgtcgcgtggaaaccagatggcacacaacttctcactgcttctggtgacaaaacctgcaggctgtgggatgttgaaaccaaatccgtggtgtctgagtttgttctgggaaatcaggtggaggaccagcaagtatcttgtctctggcaaggcccctatcttctcaccgtgtccctcagtggattcatcacttatttagatgtgaacaatccagacaaacccatccgtaccatcactggacacaacaagcctataacagcccttgctctgagtccggatagaagtacggtgtacaccggctcacatgatggcttcattacacgctggaatgccaaaactggagagaacgagcgtgtgcacggcgtgggacacgggaatcagatcaacgggatgaaggccacgggtgagctgctgtacacgtgcggcatagacgacaccatcaagcaggtggagctgacgagcaacgcgtacgggccgtatgacttgaagctggggtcccagccccggggcctggacattgacgagaacacattggtgacggtcacggtgaagcaaatcagcgtgattgaaaacggatccaaagtgtcttccctccccatccagtatgagccgtcctccatctccctggatacggaacatggactagtagcagtgggcggggctgatagtaaagtacacatctatgagctcaataataagaccttgagcccaaaaaccgaggtggaccatctcggcccagtcaccgactgcagtttctctcctaacaatgagtacctggtggcgtccgatgccaatcgcaaagtgatcctgtaccgagtacccacatttgagttggctcacaacaaagagtggggtttccacaacgccaaggtgaattgcgttgcttggtccccggattccgcccttgtagccagtggtagcttggacacgaccattattatctggagtcttgcctcacccgccaaacataccattattaaaaatgcacacccgcagagtcaaatcacgaggctccagtggctggacaatgatctgcttgtctccgtaggacaagactgcaatacgaaaatctgggagatctcaccattctaa

SEQ ID NO: 30 - DNA, 1257 bp, Bactericera cockerelli
atgacgggaaggctgccggcatgtgtcatagacgtaggaactgggtacacaaaattagggtttgctgccaacaaagagcctcagttcatcattccctctgccattgccataaaagaaactgccaaagttggagaccaagctatcaggagactcacaaaaggtgtagaagatcttgattttttcattggagatgaagcatttgatgccaaaggttactcagtcaagtacccggtgaggcatggcctggtggaggattgggacttgatggagagattcttcgagcagtgcattttcaagtacctgcgggcggaacccgaggaccactacttcctcttgacggagcccccactcaacacgccagagaatcgggagtacactgcagagatcatgttcgagtcattcaacgtgccgggcctgtacatcgccgtgcaggctgtgctggctctggcagcctcgtggaagtcgaggcccctggaggagagaatcctgacaggcattgtggtggacagcggggatggagtcacacacgtcattcctgtggcggagggctacgtgataggctcgtgcatcaagcacatcccgatcgcgggccgaaacatcacctacttcatccagtctctcctgcgggagagggagataggcatcccgcccgagcagagtctggagaccgccaagctcatcaaggagcgctactcctacatctgccccgacattgccaaggagttcgccaagtacgacgccgaccctgccaagtggatgagaaagtatgatggagtcaatcaagtaacgaagcaaccgttcgctgtggatgtaggatatgaaagatttttaggtcccgagattttcttccatcccgagttctcgaaccccgacttcaccacccctatctcggagatcgtggacacggtgattcagaactgcccgatagacgtgcgtcgcccgctctaccacaacatagtgctcagtggaggctccaccatgttccgggactttgggcggaggctgcagagagatatcaagcgagtggtggacgcgaggctgaaactgagcgaaaccttgagtggtggatacattaagcccacccccatagacgtgcaggtgatcacacaccacatgcagaggtatgcggtttggtttggcggctccatgttagcgtccacaccagaattctacgaggtgtgccacaccaaggccgcctacgaggagtatgggccgagtatctgtcggcacaaccctgtgttcggtaccatgacgtga

SEQ ID NO: 31 - DNA, 540 bp, Bactericera cockerelli
atgcctgcctaccattccactttgactgatttcaaccaatgtgttgggaacattgccatgctcccaataaagacacaatacagaggtccagctccacagttcactacaggggagcaagatatcatagaagaagcattgtactactttaaggccaatgtcttcttccgcacatatgaaattaaaagtgaagctgacagactcctgatttatatcaccctgtatataacagagtgcttgaagaaactgcagaaatgtccaaccaaagcacaaggtcaaaatgaaatgtactccctagccctagccaaatttgacattcctggagaacctggttttcctctgaactctgtctatgcaaggcctcagactcaaactgagactgaccttatgaagaactatctgacccaggttcgccaggagacaggactgcgtgtggcggatcgtgtgttcaacactccggacggaaagcccagtaaatggtggctctgctttgccaagaagaggttcatggacaaatcgctcacagccctaggccagtcttaa

SEQ ID NO: 32 - DNA, 747 bp, Bactericera cockerelli
atgaagctctttgttgttgtaaccagcgtggctacagctgggggagtggtttgctttcactttgcttccgaaatacttggtctggaccaaagttattactgcagtggccgatgtgctatcttgaattcagggcagagttgttctgaacatggcagtgacttcatcatccgagagcttcgagaacaatcattctctcctgtggaacgttctccccgtgattgtcttcttctccgtgttcttcacttcaggccgagctctgttcaacaccttgaggaagtccgaggtcttcgcccgcatgcgggagtgaatgtaggccttggaacatttgatgtggtagtgcagatagtcccggaacatgtggatcagattgatggtgttctctcgggccacccggtttgtgtgtctgggaaacagcacaaacgtaatgtagccaatgttgtccccttgtcgggcgtccgtgtctctcagctccaggggcggctccttgtgactgaacagcacctggggcgccgtgtgactcgcccgccgcccctccttgaactcctgcatgaacaccttgcctatgatgacgtcatcctcaccccggaatactgtgctgaacaccaccgtcacgcggtctgccttcgcctccacatacatagtttcttcattcctatagttgatcacggctctactctgaccttcctctcctctttcttgaaagtcaaagtatttttcaaacacagatgcaaaacaattcctctttaa

SEQ ID NO: 33 - DNA, 1269 bp, Bactericera cockerelli
atggaggacccaggatacaagaaagccacaagggttttcaagaagagttctccaaatggaaagatcactgtgtacctagctaaacgtgactttgttgatcatgttactcatgtggatcccatagatggtgtggtcctcattgactcagactatctaaaggacaggaaggtttttggtcatgttctggcagcattccgctacggtagagaggacttagatgttctgggcttaacattccgtaaagaactcttcgtgacatccgaccaaatatttccacctcttaataccccaaccacaaacaaaccattgaccaggctacaagagcggctaatgaagaagctgggtccgaacgcttttccttttttcttcgagctacctccgtcctgccccgcctcagtcacactacagcccgcccctggggacacaggcaaaccctgcggagttgactacgaactgaaagcttttgtgggagatacagctgaagataaaatacacaagagaaattcagtgaggctagcaataagaaaaatcatgtatgctccaagtaagcaaggagaacagccttccgtagaagttagcaaggaattcatgatgagtcccaacaagttgcatttggaggcgtccctagataaagagctgtattaccacggagaaagcattgctgtgaacgttcatgtagctaataattcaaataggacggtcaagaaaatcaaagtttctgtgaggcagtttgcagacatctgtttattttcaactgcccagtacaaatgtaccgttgctgagacagagagcgaggcgggctgtcccgtgagccccgggttcacgctcagcaaagtgttcgcgctcaagcccactctggactgtaacaagctgaagcgcggcctcgcgctggatgggcaactcaaggacgaggacacaaacctcgcctcgtcaaccataatcaaagatcccatggctagggaaagtttgggcattatagtgcagtacaaagtcaaagtaaaactgtgtcttggagctctgggaggagacttggtggcagaacttccatttacactgatgcaccccaaacctgatgatgaagaacttatccccgcatgctccccttcaggaaatgaatctaatgagttaaaatccaacgacaagatactcgaagctaacctcatacaattagatgacgcgtgttccccagaactaaagaacgaggatgatattatatttgaagactttgccagatttaggctgaaagcaggtggggagacggatgcctag

SEQ ID NO: 34 - DNA, 444 bp, Bactericera cockerelli
gcttcaggcgtaacggtgtccgacatttgcaagaccacctacgaggagatcaagaaggacaagaagcaccgctacgtggtgttcttcattcgcgacgagaagcagatcgacgtggagtacattggcgaccgcaacgccacctacgactcgttcctggaggatctgcagaaggcgggcaccggggagtgtcgctacggcctgttcgacttcgagtacacgcaccagtgtcaaggcaccaccgaggcttctaaaaaacagaaactgtttctaatgtcctggtgtcccgacactgccaaggtgaagaagaagatgttgtactcttccagctttgacgctctcaagaaatctctggttggagtccagaaatacattcaggctaccgatgcatccgaagcctcggaggaagcagtggaggagaagctacgagccaccgacagacagtag

SEQ ID NO: 35 - DNA, 1236 bp, Bactericera cockerelli
atgattagcacgatttcagtgaccgttgcattatttggcgcctgtctggctgccagcatagcacccacccccaccatcaagaacagtgtggtccatcccgccttcgtcaacgccggcagagttcctggcctcaagatctggagaatagagaaattcgaaccagtgcccgtaccagaaaagagctatggcaaattctactccggtgattcatacattgttttaaataccaaggaagaaaagggaaacaagaagaaaaccttctcatacgacatccactactggctgggcaaggaaacttctcaggatgaatctggagcggccgctatcctgactgtggacctggacgatagtctggggggaggtcctgtgcagcacagggaagtggaggaacatgagagccagctgtttctatcctacttcaaacccggagtccgttacatgcctggaggcgtgtcttccggtttcaaccatgtcgacatcaacgcgcctggggagaagaaactctaccaaatcaagggcaagaaaaacatcagagtccgccaggtggctctgacggtcggctcaatgaacaagggtgactgtttcgctctggacacgggcaaggaagtgctggtctatgtcgggtccaaagccgccagaaccgagcggctgaagtccatcagtgtggctaaccagattcgcgaccaggaccacaacggacgcgccaccatttctatcattgatgaaaacagcacgcctgtagacgtggccagattcttcactgagcttggttccgggtccaacagtcaggtggcagatgtgccctatggtggcgatgacgcggagttcgaaaccaaacaagataaagctgtcaagttgtacaaaatcagtgactccaccggcgccattaagtctgacgtcatagaacaaacccctctggcacagaagtcattgaatccaggcgacgcgttcatcctggacactgtcacctcagggatcttcgtgtggataggcaagggatccaccacagcggagaaggtggagagtctgaagcgaggacaagccttcctgacgaacaacaactatcctgcctggaccaagctgtctcgagttgtgcaaggcgctgagcccaccgctttccggcagtacttctcagactggagggatcaagacttcctgggaggactgggaggggggaagggaggcagtcccagtgagccagccaagaaagaagagaagaaattcagatccggataa

SEQ ID NO: 36 - DNA, 579 bp, Bactericera cockerelli
atggcagctatacggaagaagttggtcatcgttggtgatggcgcttgtggtaagacctgtcttttgattgtgttcagtaaggatcagtttccagaggtttacgtcccgactgtgtttgaaaactacgtcgctgacatagaagtggacgggaaacaagtggagctggctctgtgggacacggctgggcaagaggactatgacagactgaggcccctttcctaccctgacactgatgtcatcctgatgtgtttctccattgactcccctgacagcttagagaacataccagagaagtggacacccgaggtgaaacacttctgtcccaatgttccaatcattctggttggaaacaaaaaggatttgagaaatgatcccaacacaattaaagagctgagcaaaatgaagcaggaacccgtgaagcctgaggagggccgtgccatggctcaaaaaatcaatgcatttgcctacttggagtgttccgccaaaagtaaagaaggtgtgcgtgaagtgtttgaaacagcaacacgagctgctcttcaagttaaaaagaagaagaagggccgctgtagactcttgtag

SEQ ID NO: 37 - DNA, 576 bp, Bactericera cockerelli
atgcaaacaatcaagtgtgtagttgtgggagatggagctgtgggtaaaacttgtctgcttatttcatacacaacaaacaaattcccctcagagtatgttcccactgtgtttgacaattatgctgtcactgtcatgattggaggagaaccatacactttaggtctgtttgacacagcgggtcaggaagactatgacaggctgcgtcccctgagctacccccagaccgatgtatttcttgtgtgtttctctgttgtctccccttcctctttcgagaacgtgaaagaaaagtgggtgccagagattacacatcattgccagaagactccattcctgctggtgggaacacagattgatctgcgagaagatgcacccaccctggagaaactggccaagaacaaacagaaacccatctcatttgaacaaggggaaaagttggcaaaagaattgaaagctgtgaagtacgtggaatgctcagccctcactcagaaaggtttgaaaaacgtatttgacgaggctattcttgcagcgttagaacctcctgagactcccaagaagagaaagtgtttcatcttgtaa

SEQ ID NO: 38 - DNA, 2310 bp, Bactericera cockerelli
  misc_feature (368)..(369): n is a, c, g, or t
  misc_feature (2265)..(2266): n is a, c, g, or t
atgcctgtgtttcataccaagactattgaaagtattctggaaccagttgcccaacaggtatcccgacttgtcatcctccatgaagaggccgaagatggaaatgccatgccagacctggaacgtcctgtgcaggctgtcagtcgagcagtgactaatcttgtgaaggtggggaaggagacaatcaacagcagcgatgacccaatacttcgccaggacatgccctcctcattgcatcgggtggaaggtgcttcaaaacttcttgaagaggcctcagctatgctcaaaggcgatccttactcggggcctgccaggaagaagctgattgaaggctcccgcggaattctgcagggcacctcctcgctcctccnnctgcaaggcacctcctcgctcctcctgtgcttcgatgagtcagaggtgcgcaagatgatccgggagtgcaagaaggtgctagactatctggctgtggcggaggtgattgagtccatggaggatctggtgcagttcctcaaagatctgagtccatgcctgagcaaggtgtccagggaggtggatgcccgagagaaagagttgactcaccaggtccaccgggagattctcatccgctgtctggaccaggtcaagactttggcccccattctgatctgcagcatgaagatctacatccacatcatcagccagggaggcaagggggcggaggaggccgcggagaacaggaattacctgaccgcgaggatgacagatgagctgcatgagatcatccgagttctccagttgaccacctacgatgaagacgagtgggacgctgacaatctgaccgtgatgaagaaagcgcacagtgccatccagtccaagatgaggaccgcctacgattggctcgaggatccgctagctctgcggggcgggctgggcgagaagtcgctgcgtcagatcatcgagcacggcacgtcagtgggggagcgggctcttccacccgaccaggcagccatccgcaagctgtgctcggagatgacgaccatgacggacgccctgtgcgagctgcggcaggatgggaagggtgccacgccgcaggcggagtccctcgcccacagcatccaggagaagctggcctcgctgagctccggggtgggcaacgcagtggcgcggctagacaagcaggggcgggcaggcctggggggcacccagcccgcccacacggtgcaggggcggctggaccaagccagggcgtggctagcacacccggagcgggacgacggggggattggggtgcgggccatctcactcatcctggatgaggggaagaaggtggcggaaggtctccccggcgtgcagcgggcggagatcctctctctgtgtgacgaggtagaccggctggcgcgggggctgagtgagctgtgccgctcaggccaggggtccagccccgcggccagacagctggcctccagcctcggctccaagctgtctgagctcagggacaggatcagtggcgcggtggtgacgcgcgtggtcgaggatttcgtggacattggcagcccgctcaagctgttcacagaggcggtgctagcgccggaggacactccggggcgcgaggccaacttcgtagacaaagccaacaacctgtcggagtggtcccgtcgggccgccaagactggccgcatggtggcggctggcggctctggcggcaacaagaaactggccgaagctctggtggcggccgcaggacaggtggagtccctgacgccgcagctcgtgaatgcgggccgcatccgcatgacctacccgcagagcaaggccgcagacgagcactttgagaatctgcggaaccagtatgcgggctctgtgggtcggctgcgggacctgtgtgacgagaccattgacccggccgagtttgtcaagtactcggaggagcaaatgaagaagcacaccaacctgtgtgaggacggcatccagaggcatgacccgaacaagatggtggagcatacctcggccatcgcgcggctagccaaccgagtccttcaggtggccaaacaggaggccgacaacagtgaggacccggcctacgtggccgcgctcaacagagcagccaacgtgttgcagaacgcggttcctcccatggtgcacaacgccaagcaagtggctctcaacacgcgggaccccggggctgtgtccagatggagagaggccaacaaagctctgttggactctgtgggtcaggtgaggcaggctgtcaccgtggtgcctgatgtcaactctctgagccttcatganngacgagccattgtatcagaagcagatcaaatccaatgcgtatga

SEQ ID NO: 39 - DNA, 816 bp, Bactericera cockerelli
atgaaacctaacgcaacacctcctgaacagaaaacgtcaagtttgttgacacgagatgaaaatgaagctgtgttcaagcttttgggtaatagatgtcaggcgctttccacaactgtgatacagctgttcaccacagacggccctaacgataacgagtggcacaagaggtgtttcggcattctgtgccttgtcaaggataatccccgcaaatcctatttcttccgactctactgtttaacgaggagacaactggtttgggaacatgagctctataaaggcatgagctacatggccccacagaacttcctgcacacattcgaagccgaggattgtatcgtggccttcaactttgccaatgaggaagaggcccggcacatgagatacgtgattctggagaagcagaaacgattggagagaagacaccgggcctccactcagcctcgtcactcctccaccccgggcctggacagagagcgaagccgaaccctgcagccagccgccatgaccaacgggaccaaactgtcgcccgcggagagagcaagacatgtgcgatcctcgtccggcggaggaggcggaaaccggaagagggaggccaagcggcgaggcaacaagctgacgaaggcggacatatcctcccccacggggttcagacatgtgtcccacgtggggtttgaccccaacaaggggttcgacgctgtggacattcagaacagccccgagctggagatgttctttgagaaggcgggcgtctcgcagagtcagctgcaggaccgcaagacccgcgagttcatctacgacttcatctcccgcaacggc

SEQ ID NO: 40 - DNA, 426 bp, Bactericera cockerelli
  misc_feature (363)..(364): n is a, c, g, or t
atgcctctgcccaagcgagtgatagaacctgtccacgtagcccgaggcaccatccctgatgagctgtccgctagtctaccttcggaacttgaggccgccaccaatggaaccctggctaacacggttcgtcaactgtccagcctcagtcgccacgcggaggatatgttcggcgagctcacgcgggaagcccacggcatggctgtgagggccaactgcttgcaggtgcggctggaccggctggctgtcaaggtcacgcagctagacagcaccatcgaagaagtgtccctgcaagacattcatctgaagaaagcgttcaagtcggccatcgtgtttgaccaggaggtggtgtcccgcagtactatnncaagaggtggtgtctcgcagcactatgcccacagccatgctggagacctacaagcaatgtga

SEQ ID NO: 41 - DNA, 1544 bp, Bactericera cockerelli
atgtggaaatcagcagcaggttctgcaatagcaccagttgttccggacgaagatgatgactgggaaactgatcctgacttcatcaatgatgtaagtgaacaggagcagaggtggggctccaaaactataccaggttctggtagagatgctgggtctattgacatgaagcaactgcgggaagaagtggctatgtcagatgcatgctacaagcaaaagcagttagatggaggatcaaaagcttcttttggatatggaggaaaatttggtgttgagaaggataggatggatcagtcagctgtaggacatgactatgtcgcacagcttcatcaacacgagtctcagagtgattacaaaactgggtttggggggaagtttggtgtgcaaaatgatagagttgataaaagtgctctgacttgggagcacaaagaagtaatagaaaaacacacttctcaaaaagattatagttcaggatttggaggtaagtttggagtgcaaaaagatcgacaagataaatcagcagtaggatgggaccatcaagaaaagattgagaaacatgaatcacaaaaagattacgctaaaggatttggtggtaagtttggcatagaaagtgatagacaagataaatcagcagttggatgggaccatgtagagaaagtaaacaaacatcagagtcaaacagatgctaacaagggtgttgtaagttcttcaaaagtcaaagagctgatagctgccaattccaatacttccattaaagaaaatgtcaaaccaaaacctgacatcagtcatgtgaaaccatccaacctaagagcaaaatttgaaaacttggcaaaacaaacagaagaagaaagtagaaaaagaagtgaagaagaaaaagaaaaaagaaaactaaaggatcaaatagatctccaacaggctcaaaagttagaggaaagacgtctatcagaattgcaagtaaaagaagctgaagttgagagaaaaatgaatgcgcactcaaatgtgccttcttcaccaacaagtgattcaattccagtcaagtcaatactaaaacaatcatcaattgagaaaataactgttcaaaatagtaatgatgaagagaaagagaaacaaaaaatgattcaagaagaaattgagaagaaaaatgagcttgaaaaagaaagaataaagcaagaacaagaaattaaaaaaagaaaagataaagaagaaaaagatagacaagaaagagagcaacaagaaaaagaacaaaaggaaagagatgaaaatcaaaaattactccttaaaaaacagcaagaagaagatagactaaaagctgaggagcaagcaagactcttggaacaagagaggctaatggaagaattaagacttcaaggaaatgatgataacacagaggaggatttgggctatacagcaatagcactttatgattaccaagcatctgctgatgatgaaatttcttttgaccctgatgatattatcactaacattgaaatgatagatgaaggctggtggaggggcttgtgccatggacagtatgggttatttccagc

SEQ ID NO: 42 - DNA, 1506 bp, Bactericera cockerelli
atgctttctgaggccaccctcgagcacttgctcaagaactaccgctgggtcctcgtgatattcgtcctactgcctctctccctcctctacgacatctaccacagcgtaggacagctcatcacggagtactgcagggacaagagccaggaccatggcaagaaagtgaaccatgttcaaagtcaggttcgagcctggctggcaggggggcagacctcccccatgtgcaccgccagggctgggtggaagagcatgaccctgcgggagcccaagtacaaggcgaccatgttccctgtggacctgggacctctggattccatcctctctgttgacgaacacagtcatacggttctcgttgagccttacgtgaccatgggtcagctgacacgctacctcattcccaagggctggacaattcctgtggttattgagttggatgacgtcactgtaggcggcattgtgtctggacaaggcctggagtcgagctctcacaagcatggtcagtttcagaatacatgcgtctcctatgagttggtcctcagtgatgccagtgtggtccagtgtagcaaggaaaatgaccccgatcttttctacgctgtgccttggtcttatggaactctgggatttctgacggccgtcgagatacaacttattcccgttaaaaaatacgttcagctccagtatgtggctctcaagtccctgccggatctggaacatcacctgaagaaagaggcagaaaacaaaggcaacgactttgtggaagccattgtgttctccaaagaccagtctgtccttatgataggcaccttctgtgataccccggaaccaagcaaaattaaccgcctgggtcgctggtacaagccttggttctaccagcatgtgaggagttaccttagcaggaagaagtacgcggaggagtacatccctatccttgactactaccatcgcttcagcacatccttgttctgggaaatacaggacattgtccccttcgggaaccaccctctcttccgctacgccctcggctggctgatgccccccaaagtgtccctcctcaagctgacccagacccaaaccatcaagcagctgtacgacaagcatcacgtggtggaggactatctcctgcccctgggagaggtgagggcgtttctgcagcatatccatgaccaaatacaggtctaccctctctggatctgtcctttccttctcaaggatcttcccggccttgtacatccttccaagcctggggattgtctctatgtggacgtgggaatatatggagaaccaaaggcgcaggattatgacagcaagaaaaccattctggatgttgagaattatcttggcaaaattagaggatttcagatgctctacgctggttgctatgagtccagatctgagttccggcacaactatgaccacagcctttacgatagcgtcaggtccaggctggcctgtgagaaagcgtttcccgtcatatatgataaggtgaaccgtggtgtccgagattga

SEQ ID NO: 43 - DNA, 1185 bp, Bactericera cockerelli
ttgcaggacctgagtgaggtgggctacggacacctggactctgccaagaagcgtggcgcccacaaccactacacgcgcggctacgggaaatccaaggcgcacatcatcaacgaaatgcgacgggcaaggaaaatcggcaacctaccgcccgtctacccgaacggttggttcgcgttgatggaatcaagcgagctgcgacctcgggacgcgaaatacgtttcggcgctcggggagaatttcgccgtgttccgatcggaatcgggcgaggtgcatgtgctggatgcctactgcccgcatttgggcgccaacatggcgatcggtgggttcgtgcggggggactgcatcgagtgtcccttccatcagtggcagttcagcggacgcgacggccgctgtgtgaacattccttacagcggaaaagtgccggaggtggctcgtgtgaggcactggcagtcagtggaagtgaatgactttgtgttcgtgtggtaccacgcagaggaggaggacccatcctggcagccggaaccacttgacaaaatcacaagaggggactggcgctaccggggcagatcagagtacctcatcaactctcacatccaggagatcccagagaacggcggggacatcgcccatctgaacgccatccacgccccttccctcgtggcgggcagcaacctgcacgacctggagaccaccgccgcccagtccgcccgccacgtgtggcaggccacctgggagccgcatacggcggccggagaaacacacgtggccaccatgcggctgcggcatgacctgcggctactcgaccgcatcccgctgccgctcattggcatgaacgtcgaggcccgacaggtgggcccgggctacgtggaaatgatcatgaccacaagcataggtcgcctcgccatcctccagacagtgactccggtggaacctatgctgcagagagtgattcacaggatctacgccccgccccatctcttctggtacgccaacatcgttctctacggggagtgcatcatggtgagtcgagacatcatggtgtggaaccacaagacctacatagacaagcccctcctggtgaaggaagacaagaccctggcccgacacaggaggtggtacagtcagttctacaccgagaacagtccccgctacgagtccaagaaggatactctggattggtgatag

SEQ ID NO: 44 - DNA, 3183 bp, Bactericera cockerelli
atggcaacaccttatggtagccccaccccatctttgggaagaagaactccttctaaatctcaatcggaatcactgcctcctagtcaagatatcaagcccattactgatactctgaaagttcatttgcccaatggaacgttcaacaatgtcaaggttgtggatgcaatggatgtcaagggtcttattgatctggcaacatgcaaactcactgcctgtgatctttcctaccagtatgtatatgccgctaagatccatcatattccatctaaacaggacatctggctccacccagattcgaccatatccaatgtgattcacaactatgacaaagtctatcccatcaaagagtggaggtttgacgtgagaatacgacacattcccagtaatcttcacgatctcaatgagaaagacaaagttaccctctgtgttttatatgatcaggtaaaaaatgattatctcctgagtgacctatctaattgtttggattctgaagtggccatgcagttgggggtcctggaattaaggcatttcctcaaggatatgccccatgcctcgttggataaaaagtcgactcttgattatttagaacgagaagttggatttcacaagttcctaccaaagcacatagtccaaacttcaaagccaaagaccctccgcaaaacacttcagtcccattttaagaaaatcgctcatctctcagagaaagattgcataatgaggttcttcgagatcctcagatctcattacaagtttgaccaggaacttttccggtgtgcactgggttctggttggtcaattcctgtagatctagtggttggtcctgatgtcggaatctcttatgttactaatcgagcccaagagccatcaaaaatagctgacttctcaaagataaattctatccagaccatcttcacaaagacagaaagtgcagagaaagcaatgctccatctccaagtgaatggtacttcggaattactcatcataacctgtccgtcagtgagtgaggcacagtctttagcgcatcttattaacgggtattgtcgcctgattcataatgatacaagaagtctgtggatcaaaaatggttcaagaaaatattcaaagagtgaagagcataatatagatgacagtttacaatctgaggactattcagaacttgtagatgaggaaggagattactctactcctgctagtcgaaattacgagttgactcgtagccaagttgaagtgtgtgaaaaaataggggatggtcagtttggagatgtacatagaggcattttcaagccgcgtcctgataaagctgtgatagacgtagcagtgaaaacctgcaagggagattctgatttaacaacggcggaaaaattctgggaggaagcttatatcatgcaacagtttgaccaccctcacattatcaaactaataggtgtgtgttctgaaagtcccatctggattgttatggagctggctagactaggcgaactcagatcttatctccaattaaataagtctcgtctagatctagccacgctacttctgtacgcttttcaactttctacagcgctatcttaccttgagtcaaagaagtttgttcacagggatatcgcagcacgtaatgttctagtctcttctgacacttgtgtgaaactagctgattttggcttatctcggtgggtatctgatcaaagctactacaaggcgtccaaaggaaagcttcccatcaagtggatgtcgcccgagtcgatcaactttcgacgctttacgactgcaagtgacgtctggatgtttggcgtatgcatgtgggaaattctaatgctaggagtgaaacctttccaaggtgtaaaaaacagtgaggtgaccggcaagctcgacaacggagaaagactcgctcttccactcaactgtcccccgaggctgtacagtttgatgtcgcaatgctgggcctacgaaccttcaaaaagaccctcgtttaaacagatcaaacaggtgttgaatgaaatcttattagaagaacgacatcagcttcaagaaaatatgaaaagagaaaataggagggtctttggcatttcttggggcttgagtggctctgatgaccctcctccgcctcccaagccttcgcgatacccagacgtcatctccaccgaggctccgtcttcctgcgacagcataccacagacctacattgttgctcagaacccggaagtcctggtgcaacttctccgcgagaatgaaagccgcggtgtgaatccttctgcctacacaacgccagcttctgcgttcaacacattagcgagtgaatcatcagtattgtatggtgacagtttagtgctgagaagaggaaccaccggcagtgagtcaaatacagagtcagaagagattgagaggaggttgcgtcagcagcagttagattctgaagaagactccaggtggcttgccgaagaggaaatcaacctgaaaaagcgactgtcgatagcagccagtatatcagattctgactccttagatgggaaaggctcttgcacaccagctcaacacgcacatagttttaactccttggaaagatttcaaaatcccgatgaacgagtagttgtagtcaaaaaaatggagacaacttccactgcaggcattgatagaaccaatgacatagtgtacgaatgtaccacaaccgtagtgaaagccattatgtctctctctcaaagtgtacagcagaatcatactgagcaatatttagagttagtcaagcgagttggaacagaacttcggaaccttctcacgagtgtggataatctagtgataataatacctccatctgcgcacaaggagattgaggtagcgcacaaggtactcagcaaagatatgggtgacctggttgcctgtatgaagctagcacataactattcaaatacaagcctagacaacgtttatagaaaaaaaatgttggcggcggctcatgcgctggctatggatgctaagaatctcctcgatgtggtcgactcagtacgaaagcgacacccgacgacccagggtaatgccgccatttctagtacgaaccaccatccaaatgcagctagcgtttaccatagtaatattacatcattggacaaggcacccagtccaatgagtaacgatcaagaagaagacccaccaccccctgcccccgagtcttgcagaccaatctcgatgtag

SEQ ID NO: 45 - DNA, 579 bp, Bactericera cockerelli
atgcaagcaatcaagtgcgttgtggtaggagatggtgccgtgggtaagacctgtctcctcatcagctacaccaccaacgccttccccggcgagtacatccccaccgtattcgacaactattcggccaatgtgatggtggacgggaagcccatcaacctcggcctctgggatacggccggccaagaggactacgaccgacttcggcccctctcctacccgcagactgacgtgttccaaatctgtttctcgctcgtgaacccggcctcgttcgagaacgtgcgagccaagtggtaccccgaggtgcggcaccactgccccaacacgcccatcattctggtgggcaccaaactggacctgcgggacgacaaggagaccattgagaagctcaaggagaagaaactggcgcccatcacatacccccagggcctgtccatggcgaaggagatcggggccgtcaagtatctggaatgttcggccctcactcagaagggcctgaaaactgtgtttgacgaggccatccgcgcagtgctgtgtcccgtacctactgttcccaagaagaaacggtgcgcgatcctgtaa

SEQ ID NO: 46 - DNA, 576 bp, Bactericera cockerelli
atggcagaatacaaacttgtagttgtaggggctggtggtgtagggaaaagtgctctaactatccaactaattcaaaaccattttgtggatgaatatgacccaactattgaagattcctacagaaaacaggttgtcatagacggcgaaacagctttactagatattctagacactgcagggcaggaagaatatagtgcaatgagagatcaatacatgagaacaggggaaggttttctcctggtgtttgcagtaaatagtatgaaatcttttgaagacattggctcctatagagaacaaattaagcgtgtgaaagatgctgaagaagtacccatggtattggtaggtaacaaatgtgatttaagcacttgggctgttgacatgaatcaagcacaagaacttgcagaacaattcaacattcccttcatcaaaacttcagccaaaacacgtatgggagttgatgatgctttctacacacttgtccgagaaatcaagaaagataagatgctccgaggtaaagaaaagaagaagcgaggaatcagtggaaacaaactgaagcaatgttgtgtactactttaa

SEQ ID NO: 47 - DNA, 1082 bp, Bactericera cockerelli
atggtggggagacctatcattagagctgtcaacaaaattggtgatattgaagttaaggacctaatggtaggagatgaagccagtgctctgaggtcaatgttagaagtgaactacccgatggagaatggtatagtcagaaactgggaagacatgtgccacgtttgggactacactttcggccctcaaaagatgaatattgatcccaaagagtgcaaaattctcctcacagaacctcccatgaatcctataaagaatagagaaaaaatgattgaggtcatgttcgagaagtatggctttcactctacttacattgccattcaagccatgcttactctgtatgcccaaggcctgctctcaggagttgttgtggattccgtgatggagtcacacatatatgtcctgtgtatgaacagtttgcactgccccatctgacaaggaggttagacatcgcaggaagagatatcactcgctatctgatcaagctccttctcctcagaggctacgcgttcaaccactccgctgacttcgagactgtgaggatcatgaaggagaaactctgctacattggatacaacatcgagacggaacagaaactagccctcgagaccactgttctcgttgaaccttacacgcttcctgatggacgcaccataaaagttggaggtgaaagatttgaagccccggaaattctgttccaacctcacctaatcaatgtcgaaggacaaggcattgctgagttggtgttcaacacgatacaggctgccgacatcgacgtgaggactcagctatacaagcacatcgtcctgtctggaggctccaccatgtatcctggcctgccttctcgcctggagagagaaataaaacaactctacctggagcgggtactcaagaatgatatcgacaagttgtcgaaattcaagatccgcattgaggatccgccgcggaggaaagacatggtgttcatcggaggggcagtgctggcggaggtgatgaaggatagggacgccttctggatgagcaacgcggagtatcaggagaggggcgtgtctgttctgagcaaactgggcaaccgagccgagtga

SEQ ID NO: 48 - DNA, 1377 bp, Bactericera cockerelli
atgatacagaagtctccatctatacaaatcaggatgacttcactaggattatggagtctggaaggtaccccaggtatcttggaagtctataatcatgtagactccccaccagaagaccaaggagatgacacgagcagccacacgagagcggaggttgcctccaaactattggaaacacttgacaatttcaactatgatgaaacagaagagtcgtcggacgcctacatctccgactggctcaccggctcggaggcggctgtggctctgccctccatcttcgaagacctcgcctccctgccccccccgcctcccctgcccctagtgcccacgggccgccaagcagtctgctcctcaggcccgggcgggttctacgggccccgcggcgcatgcgcagtagtccccactgtccccgtgatcgacaagctcgggtacggagcgagtgcggcgctagtatggaactcgaataagaaggttggctacgaagcgaccgccttgtggacgccggcgggctccgatgactattccaccggctccctctactctccgcccgccgcgggcacccagtggcacctggagccggtgtaccagggcgtgtccaaccagctgacgccgccgcacagtccgcccaccatgtacgagaccagcccccgccgcccgctcgagtttgatgatctgccagcagacttgctgaaatcatctgccgccaaggagctctacgaagacctgcctgccgagtccgccctcaacagcaaggaagaaaacaacaacggccagctgatcatgtccctgctggcggagatggaccagaaggacattgacgagatcgtgcagtgctccgtggaacagaacttcggaatcgcgcctgattccgaacccaacttggaagcatccgccgcgaccaactacgtcgaatcgctgagtcccgaacactcgtgctcttcatccgaatccaacttcggctatcattccgagtccgaccgaagctcgtcgtgcagctccgaccccgactacaatccctactcgccgcccgaagtgacgcgaaaaacgcccgaaacaagccgaagtggcggtggcggacgaaaagcggcgaaaacgagcgaagccaaccgaagtgctcgagcgatcaaaccgtacgcccgcaaagcggccgtgcccgtggaagacaagaaactgcgcaagaaagagcagaacaagaacgcggccacgcgctatcgaatcaagaagaaggcggagatcgaagtcattctgggcgaggagtcggaactcagggagaagaacgaggagctgcagaagagtgtggaggatttgaatcgcgagatcaagttcatgaagaagttcatgcgcgactttttcaaaacgcaaggtgtgctcaagtga

SEQ ID NO: 49 - DNA, 981 bp, Bactericera cockerelli
atgaggaccagcgtagccctgtggcttttcctgtgttgtcagatgagcgggtgtagagaacacagctatgtgctggaggaagaggaagcaatggagaatgagctgagcagttctagagccgcccaaaacaagagcagggcgggcaagttgtactttgatgatctcttcgggatcaacctgggagacatcagtggcggagacgagaaggatagactcggcaactgcacgtgtcagtgcggggtaactaaccaggaggtccgaatcgtcggcgggcgccccactggcgtcaacaagtatccctgggtggcgcggctcgtgtatgatggccagttccattgcggcgcctcgctcatcaatgaggactacgttctgacggccgcccactgcgtgcgcagactcaaacgttccaagatccgcatcatcctgggtgactacgatcagttctcgaccacggaaacacccgaaccaacacaaatgcgagctgtctcttcaatcgtgcgtcatcggcacttcgacgtgaacagctacaaccacgacatcgccttgctcaagctacgcaagcccgtggcattcagcaagagtgtgcgtcccatatgtcttcctcctgacaatatcgacccgtccggcaagatgggcaccgtggtgggctgggggcggacctcggagggcggcagtttggcgtcccaggcactggaggtgcaggtgcccatcctgagtcctggccagtgcaagggcatgaagtacaagccgagccgcatcacggccaacatgctgtgtgcgggccgcggggagaccgactcgtgtcagggggacagtggaggtcccttgatcatcagtgaggtgggccgccacgagcttgtagggattgtgtcctggggtgtgggctgtggcaggcccggctaccctggagtctacacccgggtcaaccgctacctgccctgggtgaagcgcaatatgaaagacacctgcctgtgtgtcagc

SEQ ID NO: 50 - DNA, 1848 bp, Diaphorina citri
atgtcaactgcattaggaaaaatggctgatgaagatagggaaggaaggtttgggtttgtgtatgcagtctcaggtcctgtggtaactgcagaaaaaatgtcaggatctgctatgtacgaactggtacgagtgggatactttgaactggtgggagaaatcatcagattagagggtgacatggctactattcaagtgtacgaagaaacatcgggtgtgactgtcggtgaccctgtgctaaggacaggcaaacccttatctgttgagcttggtcctggtatcctgggaagtatatttgatggtattcagcgtccattgaaagacatcaatgagttatctcagagcatttacatccccaagggtgtcaacattcctgcattgaacagagatgttagttgggagttcaacccaatgaatctgaagattggaagtcatatcactggtggagatcagtatggtcttgttcatgagaatacacttgtcaagcacaagatgatcatgccccctaaagctaagggtactgtcacttacattgcacctgctggtaactataaagttgatgaagttgttcttgaaactgaatttgatggagagaagagtaaatacaccatggttcaagtatggcctgtccgtcagccccgccctgtcacagaaaagctacctgctaactatccactattgacaggtcaaagagttcttgattccctcttcccctgtgttcttggaggaaccactgccattccaggtgcctttggttgtggtaaaactgtgatttcccaggctttgtccaaatattccaactcagatgttattgtgtatgtaggatgtggagagcgaggtaatgaaatggcagaggtactgagagatttccctgagcttaccattgaagttgatggagttaccgaatcaatcatgaagcgtactacacttgtagccaacacatctaacatgcctgtagctgcccgagaggcttctatctacactggtatcacactgtctgagtacttcagagacatgggttacaatgtgtccatgatggctgactctacatcccgttgggctgaggctttgagagaaatttcaggacgtcttgctgagatgcctgctgacagtggttatcctgcctacctaggagccagactggcctcattctatgaacgtgctggcagagtcaaatgcttgggtaacccagacagagaaggttctgtgagtattgtgggtgctgtatctccccctggtggagatttctccgaccctgtcacttctgctactcttggtattgtccaagtgttctggggtcttgacaagaaacttgcacagaggaaacatttcccctctatcaactggctcatctcttacagtaaatacatgagagccttggatgacttctatgataaaaatcatccggagtttgtacctctgagaaccaaggtaaaggaaatccttcaagaagaagaagatttatcagaaattgtgcaactggttggtaaagcctccttggcagaatctgataaaatcaccttggaagttgccaagttgctgaaagatgatttccttcagcaaaacagttactcaccctatgacaggttctgtcccttctacaaaactgtgggaatgctgcgtaacatgattgctttctatgatatgtcccgccatgctgttgagtctactgctcagtcagaaaacaaaatcacatggtctgtcatcagagacagcatgagcaacattctgtaccaactttcctccatgaaattcaaagaccctgtcaaggatggagaagctaaaaccagagcagactttgatcaactgtatgaagacattcagcaagcattccgtaacttagaagactaa

SEQ ID NO: 51 - DNA, 1490 bp, Diaphorina citri
tgtcgataagctcgaagcaagctttacgggagaatgtgctagctgtgacgcgggactacatatcgcagccacgaataacatacaaaactgtgtctggtgtcaacggacctctggtcatcttagacgaggtgaaattccccaagtacgctgaaattgtgcagctccgtctgaatgatggatcttaccgtgccggacaagtgctggaagtcagtggctccaaggctgtggtccaggtgtttgaaggtacatctggtattgatgctaagaacacagtttgtgagttcactggagacatcttgagaactcctgtgtcagaggatatgttaggacgagtgttcaacggcagtggaaaacccattgataaaggacctcccatcctagccgaggactacttggacattgaaggtcaacccatcaacccgtacagcagaacctacccgcaggaaatgatacagactggtatctcagctatcgatgtgatgaactctattgctcgtggacagaagattcccatcttctctgctgctggtctgccccacaatgaaattgctgctcagatttgtagacaagctggtctggtaaagatgccgggtaaatctgtacttgatgattctgaagataactttgctattgtgtttgccgctatgggagtcaacatggaaactgcccgattcttcaaacaagatttcgaagaaaacggttccatggagaacgtgtgtctgttcttgaacttggccaacgaccctaccattgaacgtatcatcacacctcgacttgccctcaccacagcagagtttttggcgtaccagtgtgagaaacacgtgttggttatccttacggatatgtcctcttatgctgaagctttgcgtgaggtgtcagctgcccgtgaagaagtaccagggcgacgtgggttccccggatacatgtacaccaacttggctaccatctatgagcgtgctgggagagtggagggcaggaacggatcgatcactcagatccctatccttactatgcctaacgatgatatcacccatcctatacccgatttgaccgggtacattaccgagggtcaaatctacgtcgaccgacagctgcacaaccgacagatctatcctccaatcaacgtgctcccctctctatcccgtctgatgaagtctgctattggtgaagacatgactcgcaaggaccattcagatgtgtccaatcagctgtacgcttgttacgctattggtaaggatgtgcaggccatgaaggctgtagtaggagaagaagctttgactccagatgacttgctgtacttggagttcctcaccaagttcgaaaagaacttcgtgtcacaaggtaactacgagaaccgcacggtgtacgagtccctggacatcggctggcagctgctccgtatcttccccaaggagatgctcaagcgtattcccgcctccacgctcgccgaattctatccccgggattcacgccacactggcgccaagtaa

SEQ ID NO: 52 - DNA, 732 bp, Diaphorina citri
atgtcaggtaaagagagactacccatattcccttcccgaggagctcagtcactcatgaaatctcgtctgaagggagctcagaagggacacagtcttctcaagaagaaggctgatgctctacagatgaggttccgaatgattctcagcaagattattgagactaaaactctcatgggagaggtaatgaaagaagctgcgttctctctagcagaggccaagttcactactggagatttcaaccaggtagttctacaaaatgtcaccaaggcacagatcaaaatccgcactaagaaagacaatgtagcaggtgttactctacccgtgtttgagagctatcaagatggcacagacacttacgaactggctggtctagctaggggtggacagcagctggcaaagctgaagaaaaattaccagacagctataaaactcctcgtagaacttgcttcactacaaacatcatttgtaacattagatgatgtcatcaaaattactaatcggagagtaaatgccattgagcatgtcattattcctcgcattgaaaagacactagcatacattatttccgagctggatgagttggaaagagaagagttctaccgtttgaagaaaattcaagacaagaagaaggtgatcaaagcagcttctgaagcttttaggaagtctcgtaaatatgatgaagaacaggcattcaatatgctagaagaggaagatcaggacattttattctag

SEQ ID NO: 53 - DNA, 819 bp, Diaphorina citri
atggtccaaaatgtattacagagcaacaaagttctgaaagattttagtcttaaactttgcgacaaatctacattccggtcgtacctgaaacttccgaacgcagccgacattttccgaccgcccccgacacttctcggcatggcgttagacgacgcagcggtggaaaagcaaatcgaacgcatggtggcgtttatccaaaccgaggcggacgaaaagttggacgacatccggcgaaaaatcgaagaggactaccagatcgaaagagagcgagtaacgcgagacggaaaggcgagcgtggatgaagaatatgctaagaaataccgccaggtagagcttcggcacaggacggactgttccaatattaagagcgaaggacggatgaatgtcatgagggtgaaagaagacagtgttggaaagatcatcgaggaagcaaaaggcagactgtctgacattacagaggatcgcacaaagtacaccgagatactcgaaaaactcattttccaaggtgtcctcaagttactggaacccactgttctgattcgatgtaaagaaaacgacctgtgcattgtgaggcaactactgccgctagtagctagggactttgagaatattacgggatccaatcttacgctgctgatcgattctgaacagtacttaccacctgagatttcaggaggcgtagagcttatcacccccgatgagaagatcagagtgaacaatacgttggaaatgcgactagaactgatggccctacaagctctgcccgatatgagagatatattgtatggtcctaatccgaataggaagttcgacgattga

SEQ ID NO: 54 - DNA, 8469 bp, Diaphorina citri
atggcttgcgatcgttgtgttcaactttcatcggaagttgaaaaattgaccactcaaaataagaagattgtacaattgctgaatgtgttaaatgcctccaagaaacacaaagaacaactgatagaacataccttggattacatcaagtccaaagatgaagactacaagaaggaaaagatcaaaatcaaagaacttgcaatcaaagtcaagaacaccaccaaagaatcggttctcaggaatgtacccaagcagattgatgagtgcagtgcactctacatcaataaaactgctgagatacagaattacattagagccacagagattgccagagctgccaagcaagagttggaaacaaagagccttgaattggaaggaaacaaaatgtacgtgaagcaactgattgccaaagttgatgagctagaacataacaagaggactttgacggaatccttggcatcaactaaactgctcgtcacaaaagctttgatgagaaacaagaagatagagcttgagatgagaatgctcaatgatgctcactcatatcataacagagtggaaaccggagtgaagaaagctttgcgtagattggaatcatcagatccaaattccacaaagagctgtatgaggatcctgaataatctagttcatttctttgaaaaagaaccactctttgtgaaacgatctcggaaattgaagaatggtgcggataataagggcatagtcagtcctcacagtgtatcttctggttatagcagtggaactgcaagtccttggtctactccttacaatactcctcaatcctctgaggaacctaacactcagttcacatttaccttcacaccggacggggattgtgtgctctcccctcttccggctcagtcacagtcaaacccttcaacttcatcctcatcaacccccaccaccaggtcttccattacttcaccattggttcaaccatctccacctcattccagctatgaaccaagtcagaaacttagttctgagaaagtcgcaacctcttggtcaatgtcttgtgatagttcggatgatgaaattgttgatggagctgatgatcgggatagtgatgttgaaagtgatcgagagacctctataaaaaacacttgtaaattagaaaccaataaacacaaaagtaaagaccttggtgatttagcaagtgacaaagtcaaggaaactagggaattacaaagtgataaaggcaagtcgtcaccaaaaagaggtgaacttgacagtgatatggttgaaattggtgagcggagcaagaaaccatccagtgataatgtacaaagtacatttgatcttgttgagccaaatgacatgtgtgaaagtaacaaaatcgattgtgataatagtgatgaagggaagagcagtgatatccaggagataactatagcaagtgaagtagccagtcccaaacatgcttctgcagagttactacaccttcctgtccttgagaattttgaagatgaaaaacctataaaacaattacctggagttacaagtgcagaaaaggatcaagcccttccaaagcaaactgttcaagctaatcatagtccagaagctaaaaaacatttctttggttcgtataagcagagtttgttttcaccttcaaagacaccaccgggaaagaaggaaacaaaacaggagattgcaagggaaattgccaaagcggccaaacaaatagaaatgagtctctgtgaagaagagaattgtgtgaaggagaagaagaaacgtgtatcacgaagtgtgaagagaaggagctctaaaaagtctataggcgatgatgaaactgacgcagaaagtgctactgtggttgcaaaagaaaagaaaaatggaacgtctgaaatgagctcagaagatgacagcttggaaaagttgcgtgtgttgagaaaatctcgtaaatcaattgacaaagctctcaaggataatgtcggtagtactaagtcttctggaaaatcgccaagacgtagatcaactagaaacaaaagtacagaaaccagttctattcgttcagaaactagctgtgtcaattcagaaaacaatcttattgactctaccagcacagactttgaaatttccaacctctcagacagtgattgtgaaagtactgatcttgtagacaaagctgatgagaacaccgaagaaaatgtcaaggaacttttgaaaatcgacaaggaacgacacgaacacgtgttacagatattcaaagacattgaagtgatgcaaaatgttgaactcatttcgaactttgatcttctggcttcaccctgcaagaaacaaacgcgatccagatccaacagtctagttccaaataacgaagcagataaactagaaaatgatgtaattcctaatgcatttgattgtgataaagatatcagcaatacagaacttgagaaagagaaatatgaatcactcgaagctgtatataacgaaaccaacaaaaagctcgtgaaagattctgaagaaaatgcccaagatgatgaagatgattgtaattcacaagtagaagatgatgaatgcaaaacgccctctttgagaaggagtagaagaaactcaaaacggaatgcagaagagatatccagtcctgtaactaggaagtcacctcagacccaaaacaccgagtccaatttgaaaggtaccccctctgacaacaagaagtcacgaaaatcacgcaccatcgaagaagaaaggttaccaagaacgagatcaaaatccacgcacaaggcagtatctattcctgaagtagataatttgagacaaagaagctcaagtgacgttggaacaaagaagtcctctgaagaaacggaaataccacttgccgatagtgatgaaatacctagaagagtaacacgaagaagtctatccgtcatggagaacaaagaagatgttaaatattcagaaaaaacaagaagaggacgtaataagaaggcggacaatagtttattaggagaggcaatgtcagatggtgacaaggataagattagtttcgaagataagtgtgataaaaacagtaaagacaaaccgaattctagtgactataatggaaaagatcaagttgttccaagtgctagcaatgaaattgaaagtgttgatgatcttgatgaatctatggaaataaacgatattgatgcttctgttgaaacattatcagagataactgataaatctagtggaatagacacagagaaaaatctcgtgaaacaaatagaacctgagaaagaagaaatggtagacgacgacaaatcaagtaatcttgaaagaactctattcgaaagtcgtgtagaaattacagaaagcaataaagaagatgtagaatgcaatgacagcatggtacaagatgtgcgatgtttgaagagaactcgaattgccctatctaaagtcggtggtgatgccgtgtccaaagtatccagtgatacacttgatacagtatccaaagtcagtgatgaaaacaaggcgctgtctcctccagagaaggctgttagctatcctttggaatcgaaggaaacaccagataaatctgctcccaacactgctccatcgacagctcaacctatgtcacctcagaatgataaaaaattagaaacagatgccccgaaaatacctaaaccagtttcagttcttgaggaacaatcttcgaagatatctgatccaagtcctgttctgcagttaagtaccgggagaaagttcaagattggacgcttgaaagaggtatctaacactaccaatgaaacagcagtccctaggaaagaattggccgatagcagaaatattgagactagtgttgaatccagtaaggaatcgccaagtactcccagtagtggaaaagaacatgtggaactagcaaatgaaaaagttattgttggtaaagaagatcaaatccacaagatcagatcgaacaaaaacattttgaatactaaaaatgagaaagtacttgctaataaaaaggatacagtccccgaggatgtctctacaaatgaaaatggccaaaacattctaaaccatacagtcaataaagcttcgacaaagaatactatactacctattgtcaaaactgacaacttaatgcaacaaaacagaatcatccaaagttacaattgtaacttgaacctggtcaaaccagccacagtacagaaacctcctcatacaatcgtgaatagtaagaaagtcaatagaattctccgaagaatgggaatggagggattcaacaagaagtttccattcattgacgctctccctgctactccacctcagtgtagagaaaatggaactggattcaagaactacaatgggtataacaactctactattgcgaaggaagaagttgaagagatggaagaaacagattgtttggttatggagatggaagatgacttacaagatgttagggaaccaaaagatgcagatagtgaagtatctgaacaattgagctcgcaaactacatgtgaagataaagtacctgttccagatatgacacaaccacttgcagataaagaagaagtagtagaaaaatcgatcagcttgcctaggaaagagggagaagaattaaaccaaatgccagtacctattccgaatataacagatatgacacagccacttgtggagaaagaaggaggagtggaaaagtcacccaacagatctcaaagaaaggatgaaaaattaaacttcgtcagcaaaataatggaaaacagtcttcacaaagaagatcaaatccaaaacaaaggaattgttcctgagcaaagcacaagcttccctaatgtacacgtcccagacaaagagtctactagtcccaataaaatgaataatgacttgagtcctaacaaatcagatgacacaaccagcttgacatccacaacatctagtggattgaagtctccttcaaacctgttgacatccaatgaaacaccttgccaatctttgcagtcacctccctcaatatttcccaaatgtattcaaaaccgcttggcaaagatccgttccacacacaatttaccaatttataacataactccaatgagtaatttggctcacaatactcgacctggggagaaaaccaacatgaatttaggtgttcatggactcaagtcgaatgaaatgagccctccaagtctaaacaaagtagacattcaagatgccgtcaaaccagttcaagctagtcctacagatatttccaatcaagtgtcctataacaataatccagaaccgactcttgttggaaaccaaacttttagcttctatggacagaagagatcttcggacattactgctccttttgaaaaagaaagcacggtatttagaccaccaaataacttcagtaatactaacacggtttctagaccatcaaataatgtcacctctaatagtgacaatatggtatctagaccaccaaataacttgaacactagtttcgtcaacaatagccctcctaaggtcaacggacctttggagaattctgttcatcaggcgaatgctaccaaacccacggaaaagattgttcttccaacattatccttctacaacacacgcaattctccttcatatggtgctaacaaacgtaaatgtgaagaccctgaatatgtgagttctgatgaaagcaagaaacttagaactgagcttgaaaccagtggtacattgaagagcacggttaacacacctaaagacaaagatgaacccaagtcctatacactcattcaacgctcaattgaacatctacaatccgaaatgtccaaacccgatgtgacaccaaccaatcagatcaaaactaaaacggtgacgaaaaaactgaacctgctgtactacgccatgcaaccgaatgataagaacgtgttcgataggaaaatcaaagcgtttgtcaaagagctggaagattacccgctggatactttatccaaacgaatcatcgcgttgtacgataacaaacagctggtagacaatcccaaagcggtgacaaacattcaaaccaaagtgttctgtctgattatacagctaaacacaacacggcctgagttcctacccacgtttatgaagaccctgcatgagacactggtcctgcatagtgagaccacactcctatctatcctgtatgcattgacacaactctatgtaggcttgtgtcgctggcgtaatgatccagcatctgttatgacattcctctacgataccatctacttccataggaaacgctctctcaatatactagacgctttgatcacacgatggcctactgtgtatcctgtagccagtcgctgtgtcctagttaaagtaatgagttgtatcctgctcaaccagcagacatcaagccaggggagtttccttgatgaccagaatgtcttcgtgaagacaaagcagcagttgagaacaaagtacagatatcaggaacagttcacacttgccggtctactagcagagttgatgaatatgttgaagagtaaacactcctgctgctctaacccgtggcagcgaaaccctcgttccaattctcaccaccagggagcggaatcgtcgagtttcagcggttgtgtacagagggcgctggtgctgttggctaaacattggcgtccggcgccagacttgtacagtgttgtcgtggagaggaacttggcaacactgttcagaagtagtttggattgtggagcaatcagtgttgctgcctcctgcgctgaactcatgggtatgattgggaaagccatgccccgcgcctcctccctcctctgcatcgtctccctgctggatattctggaggtcggcctgcaacatgccgcctgccgctcaccctccccccttcgcacctctataggccgcgcgctaacctgcctcacggcccacaacacggagcgttgcgtggccatcctcacctcggtgccttggacagaggccgaggtcaatgaccttgtcaaggacgttgcagccaaggtcatcgcgtcttgtaagatccgagtgacaagcaccatgatttccgaatctctcctagaacacctgctcaagaagtaccgctggattttcgtcattttcttccttctgcccctgactttcttctacgatatctatcacttcatacgacagcaggtcaccgaatatttcaaggacaaatcagtatgccatgatctcaaagtaaaacatgttcaaggtcaggttcgtgagtggatcaaaaccgaccaatccataccgatgtgtacaggacgcgctgggtggaagtgtatgtcactgagagagcccaagtacaagtcgtccatgtttccagtggatctggaggcgatggataccatcctgagtgtggacgaggagaagaagacggtcaaagtagagccctatgtgaccatgggtcaattaacccgctatctaatacccagaggctggaccatccctgtcgtggtagaactcgatgacgttacagtaggaggcattgttctgggtcaaggtctggagtcaagttctcacaagtacggggtgtttcagaacacatgtgtctcctatgaactcgtactgagtgatgccagtctggttgaatgtagtgaggaggaagaccgggatttgtttcacgctataccttggtcctatggaacactgggatttctgacggctgttgaaattcaaatcattcctgtaaaaaaatatgttcagcttcaatatgtcgcgttgaaatctctgccagatctagagcatcatctgaagaaggaagcggaaaacaaggccaatgatttcgtggaagccatcttgttttccaaggaccagtccgtgctcatgataggtaccttcagtgattcgccagatccaagtaaagtgaacaacctaggccgctggtataaaccctggttctatgaacatgtaaggagctaccttaccaagaagaagttctctgaggaacatgtgcctatcctggattactaccatcggttcagcacgtctttattctgggaaatacaagatatcgtcccatttggtaaccatcctctatttcggtatctcctcggctggctgatgccccccaaagtggccctcctcaagctgacccaaacccagaccattaagaacctctatgataaacatcacgtagtacaggactacttggttcctatcgaggagcttagatcatgtgtacactactttcatgacaatattcagatttaccccctctggatttgccctttcctgctgaaagacctgcctggtttggttcatcctgctaaggcacaggatggtatgtatctagacttgggactgtatggagagcccaaagctaaagactaccacagcaagaacacaatcaccgccttggaaagctatctgggaaaaatcagagggttccaaatgctaggcgcgggtgtctaccaatcatattccgagtttcgacagaactatgaccatagcttatacgacagagtacgagccagactgggctgtgaaaagggttttcccgtcatctatgataaggtcaatagagttgcccgggactga

SEQ ID NO: 55 - DNA, 399 bp, Diaphorina citri
ttttccactgtaaggaatattcacgcagcgcccatcatgtccactgaactgccattgatgaaacggacactcaatacaatcccctctcacgaatcctccgatggcaatgttagctcccaaatgaggacagtacgcatccagcacatgcacttctcccctctccgatcggaacacagcaaagttttccccgagtgcggaaacatatttggcctgtttgggtttcagctccgaggattctagcagtgcaaaccacccgtttggatacacgggcgggaggttgcctattttgcgcgccttgcgaatgtcgttgatgagctgttttttcgttttgccgtacttgttgtagccagaccctcgtccgtccaagtaactgtagccaacttcactcagatcctgtaa

SEQ ID NO: 56 - DNA, 1305 bp, Diaphorina citri
atggcttcttcgggaatatggagtctgaaaggtaccccaggtatcttggaggtctataattatgtagactccacattagaagacaagggagatgaaggcagcaccaccaagagagcagaggttgcctccaaacttctagaaacacttgagaagttcaactacgatgaaacaacagaacccgcagattcttacatctccgactggctcggtggcgaagagaaggtcatcgaccttcccatctacgaggacttcccactgtcccaaccttccatcgttcaaccaggtagcgctaccgtcgttcaaccaaatagcgctagcatcgttcctcaaggtagcgccagtgtcgcgttccgtcaaggtcacgcgggcttcgctcaaggtcacggccaaggtcatgtgaaggtcggctacgaggcgagccccgtgtggcaggccgatgagtactcgagcctgtattcgcctccggccgcgcaatggccggtgtataacgagatggtggccgcggtgtcgagtcagctgaccccgccccacagtcccaacatgtacgagctgagccccaaacacgcggccgagtttaaagatctggccgatgacttgatgaaacctgcggccggcaaacttccgccctacccgtccgatatcggcctccaggccaccagcccctgtctcctcccccctcaacctgactacgaggagaatctgaaggacggaggtcagctcattctgtctctcctggccgagatgaatcccaaggacatcagtgagctggtccaagccaacgagcttattcaagaccaaggtcaaacaagcgagccctacaccgtccccgatctgagcgctccagcgagcagcggcgcaaacatgaactacagcgaggtcatccttagtccggaccactcgtgttcttccgactccaacttcgactacacctcggacacctcttccgacccggactatattccgtcggcgcgtgtcgggttccctcggaaaccgagagctcccgaagtagcttcggggaagatcggctccgctcggaaggaaacgaaacgggccaagccgtatgcgcgtaaggcggcgctacctattgaagacaagcgcctgcgcaagaaggagcagaataagaacgcggcgacgcgttataggattaagaagaaggcggagattgaggaggttctgggagaggagaaagagctgttggagaagaacgcccagctgcagaagagtgtggaagatctgagccgtgagatcaagtttatgaagaaattcatgcgggacttcttcaagaaacagggcgtgctcaaatga

SEQ ID NO: 57 - DNA, 443 bp, Bactericera cockerelli
gacgaaagcagcggcggctggtgcgagagaaccccggggtctttctggccgtctccaaaggagccaatttagccatcaacgaatgccaattccagttcaggaatcgaaggtggaactgttcgacgcggaatttcttgcgcgggaaaaatctcttcggaaaaattgtagatagaggatgcagggaaacggctttcatctacgccatcaccagcgcggccgtcacgcacacggtggcacggtcgtgcgccgaaggcagcatagaatcgtgcacgtgcgactacagtcaccagtccaaaggtcaacggccgaagaccacgctcaacaatgtcgcgggcgtgcgagattgggagtgggggggctgttcggataacatcggatttggattcaagttttcgcgcgagttcgtagacacgggcgaacgaggtcgaagtctaagggagaag

SEQ ID NO: 58 - DNA, 191 bp, Bactericera cockerelli
gtgacctcatgtgttgtgggcggggctatagaacgcaggaaatcaccgtggtggaacggtgcgcttgcgcgtttatctggtgctgtgaggtcaaatgtaaaacatgtagaacaaagaaaaccattcacacttgcttgtaaataatacgagtgagaagtattccggcctgggactagtcatccatgactttg

SEQ ID NO: 59 - DNA, 286 bp, Bactericera cockerelli
gccggtcagtatccgtacccaatcctgagtcctgatatgagccaggttgcagcgtcctggcactcccctagcatgtaccccacgatctcatcaggtggctccggattccggggtgcctacccatcctcattgcctattacaagtacaagtttacctagtgatttctatagattctcgcccaccagcttgatggcggcacaccccggactgagcccccactcccacgccgccattgttacccccgggcccaaacaagaactcctctcagaccacaaccataggtcac

SEQ ID NO: 60 - DNA, 235 bp, Bactericera cockerelli
ggtagttgatcaggttcaccacagcatgcttcagcatttgactgggctccgtcaagcgctgcacagcagtgggagctgccgtatcaaattgagttgatggaatctcaatgccttcttccagtgtctcgggaaacatagcagcccgaacccgctgactccgagtctggctcagctgagaattcatttcatccacttgatcctgggtgaacccctgtccaaagccttggtccatctc

SEQ ID NO: 61 - DNA, 230 bp, Bactericera cockerelli
gggttggatatggcttctgatatctccttggctacgtcttgctgttccgcaatgtcatccatcatatcatgcacctggttcacatccatgtgtttatgtgctgccttgagtgcatcagctgcattcttcatggtggtaagtaccgcagtgtttgtgttagcaccctccagagcttcccgttgcatctcaatagttgacagggtgccatcaatttgttgcaattgtttctc

SEQ ID NO: 62 - DNA, 178 bp, Bactericera cockerelli
gaccatgtcatcatcattcactgtcggcttggaggtctccagagatttcaagatgtggttcatggacacagggggttccaaaagtttgtcggacggcacatccatgaagttcatttccctggcgcccggggtggagggggagcaaggggtcaacaggtcgttcatcgtcttgctcggg

SEQ ID NO: 63 - DNA, 264 bp, Bactericera cockerelli
acttcagactggaaacgaggcactgaagaaagtgaatgagctcatcagcatagaagatgtagagaggattctcgatgagactcgggagagtattgagaagcaacgggagattgatgagatgctgcaaggtgttctaacaactgaggatgaggaggatgttgagaaggaatacgagaagatgatggcagactccttggtgcctcagccggaacccagagtccccatcgcggagccagaggagtttgtcagcttacctgaggttcc

SEQ ID NO: 64 - DNA, 310 bp, Bactericera cockerelli
catcgggtgtaactgttggtgaccctgtgttgaggacaggcaaacccttatctgtagagcttggtcctggtatcctgggtagtatttttgatggtatccaacgtccactgaaggacatttgtgagttgtctcagagcatttacatcccaaaaggagttaacattcctgccttgaacagagatgttagctgggagttcaatccaatgaacttaaagattggtagtcacatgaccggtggagatcagtatggtattgtacatgagaatacacttgttaaacataaaatgatcatgccacccaaagcaaaggg

SEQ ID NO: 65 - DNA, 240 bp, Bactericera cockerelli
cagctccgtttgaacgatggatcttaccgtgctggtcaagttctggaagtcagtggctctaaagctgtggtccaggtatttgaaggtacctctggaattgatgcgaagaacactgtctgtgagttcacgggggatatcttgagaacaccagtgtctgaagacatgttggggcgtgtgttcaacggaagtggaaagcctattgacaaaggaccccctatcctagctgaagactaccttgac

SEQ ID NO: 66 - DNA, 264 bp, Bactericera cockerelli
tcatgaagtcccgtctcaagggggctcagaagggacacagtttgcttaagaagaaagctgatgctctccagatgagattccgtatgatcctgagcaaaatcatcgagacaaaaaccctcatgggtgaagtcatgaaagaagctgccttctccttagcagaggcgaaattcacaacaggggatttcaatcaggtagtcctacaaaatgtaaccaaggcacaaatcaaaatacgcactaagaaagacaatgttgcgggtgttactc

SEQ ID NO: 67 - DNA, 272 bp, Bactericera cockerelli
gagccaagagctcaatacctcctgtggtattaacaggtaagaattgttcactgtcaagtttcaagttaacttccttaccagcaacatcttggtattgcttggccaccgtaggtaagacaccattgacaagttctttgtcagcttcccgggcacggatcagaacattgggctccaataattgcagcaggccttgaaccattaacttttcaatgagttgggtgtacttggggcggtccttggacacctcacccagtctgttcctggcctcttcc

SEQ ID NO: 68 - DNA, 247 bp, Bactericera cockerelli
ccttgaactgctggaggcaatccagaaacgcgaccatggccatgtcaaatttggtgtcccagaagaatttggcacccccgctagcaaacagggggagatttttgtgctcggcagtatcctcaatgtaagagtggttgccgtagggcactatgcggtaccgctggaaggtgaggttgagtttcctggccagggctgtgagtaagagggccgtctgtccccaggccgcgttaatttcgctccagtccac

SEQ ID NO: 69 - DNA, 264 bp, Bactericera cockerelli
gcgtaagattgtcgtaggcggtctgtgtcatctgttcgcggagagtttgctgtgcgtgttggtagagtggcgacggatcctccatatgagggaaagccctaggaatacggaagttattgctcatagaaccatacatttgctgatatcttattgccttctggatctggtgaatggtatacttggcatggatcaagcgcggattgctatcgtcggcgcacacatcatacacattggtccaggccgatttcgaaggcgtttgccaga

SEQ ID NO: 70 - DNA, 269 bp, Bactericera cockerelli
ctaacaacagagccagcattgaagtccacggaggagacaaactcagagtttactgtggacacaagaacttcaaggagctgaaatctcactcggacgactccctcttggttcaatgcaacagtgggaaatccttcaagctcctctctactcatctgcacaatgcccagtctgtccaatattctcaatctgacaaggacgtaccttccgacttctcaactggatcctcagatctccgtcgaactccgcactccagcgcttctctagaacca

SEQ ID NO: 71 - DNA, 239 bp, Bactericera cockerelli
gctcacgttggcgtaatagtaggtggcccactgccaggccgccatcaggaagtccccatcaggagccaggtggcctcgggacatgaacatcttgctgttgatgtacttctgggcctcggcctttgaaccgaggatattttcgaaaacgcgaacctgttgtgccaagttataagccttgttcggtgggagccctttgaacattgatttgtctcccactcggaagttgggtctcttgctct

SEQ ID NO: 72 - DNA, 258 bp, Bactericera cockerelli
agaactttgaggacgcccaggagttggaggattggctgccgcccgacttcacggaccggcccgccctcatcagtctggtggcggactggaagtaccagaagtggctgcaactgctcaaccagatctggaagcagctgggcaagaagatgaacatcgatgtgctggtcaacgccgaccggcactcactcatctatgttgagaacggcttcttcattccaggaggacgctttctggagctgtactactgggacacctact

SEQ ID NO: 73 - DNA, 245 bp, Bactericera cockerelli
ggtcacggctaaatacacacctctggattggtggcagacgtcaatcgtgtatcaggtgtatccgcgatcattcaaagatgtcaacggagacggtattggagacttgaaaggtatcatgcacaagataggctacttgaagaacctgggagttggcgccatctggatttcgcccatctacaagtctccgatggccgactttggctacgacatctcggactttcgggacatcgaacctgtgttcggca

SEQ ID NO: 74 - DNA, 239 bp, Bactericera cockerelli
acttcgacaaccgtctggtgtcacatctcgcggaggagttcaagagaaagtacaagaaagacatgagtggcaacccccgggcgctgagacgcctgcgcacagcagctgagagagccaagcgcacgctttcctcaagcacggaggccagtctcgagattgacgcgctgtacgacggcgtggatttctacacaaagatctcccgcgcgcggttcgaagagttgtgcgcggatctattccgc

SEQ ID NO: 75 - DNA, 253 bp, Bactericera cockerelli
gaaagcgctgcaatggctaagaggtgacgacgcagacatcagtagagagttcgcagagattgagaagatgaacaatgacggcaatgagggggatgacgagagttccacagggtgcagtgaggtgttcaaagcaatgtacatgaggccactcctcatcagcataggactcatgttcttccagcagatgagtggcatcaacgcagtcatcttctacacggtgaaaattttcaaggacgctggcagtaccattgac

SEQ ID NO: 76 - DNA, 223 bp, Bactericera cockerelli
tgcttatgtacaacagtgctgacgagggtttcgacagcacttatcccaccaatgtggtggtccggagcaatggtagctgcgtgtacattccaccgggcatcttcaagagcacgtgcaagatcgacatcacgtggttcccgttcgatgaccagcggtgcgaaatgaagttcggcagctggacgtacgatggattcaaggttgatcttcgccacatggatgagaa

SEQ ID NO: 77 - DNA, 300 bp, Bactericera cockerelli
tctgagttgtgtcagcctgctggagcagcccgccggcacgccgcccaatatcgtcctggtcgccttcacgttcggtctcgtcatcttcacgtccgtccaggctctgggtcacgtgagcggcggacacttcaaccccgccgtgacggtgggcatgctcgccacgggcaatgtgagcgtcatccgcggcgtgttctacgtggtggcgcagtgcctgggtgccatcgccggcagcctcatcctcaagtccctcacaccggtcgacttccagggcaacctgggcatgaccacgctcaacaagca

SEQ ID NO: 78 - DNA, 273 bp, Bactericera cockerelli
agtcaccaatgctgtgtcctggaggtccgagggcatcaagtacaggaagaacgaggtgttcctggatgtcattgagagcgtgaacctcctggccaactcgaatggcaatgtcctacgcagtgaaatagtaggtgctatcaagatgcgggtgtacctgtcaggtatgccagaactccggctgggactcaatgataaggttctgttcgagagtacaggacgaggcaagtccaagtctgtggagctggaggatgtcaagttccaccagtgtgtcag

SEQ ID NO: 79 - DNA, 260 bp, Bactericera cockerelli
tctgacaagttcatctgcgtgcgagagaagatcgcggattccgcgcaggtggtcatcatcgacatgaacgaccccaccacccccatccggagacccatcagtgccgactccgccatcatgaacccagccagcaaggtgatagctctcaagggcaaggctggcaacgacaataatcccaacgcgcccaagacgctgcagatcttcaacatcgagatgaagtcgaagatgaaggctcaccccatgaccgacgacgtggtctt

SEQ ID NO: 80 - DNA, 232 bp, Bactericera cockerelli
ccaagcaagagctagaagaatggtatcaccatcatgcagaactcattgctaaaacgaaagctgccaataggaatgcagaaaagcagtttgtggcggaggcagacgacatagagcccgggacagagtgggagcgcatcgccaagctctgtgacttcaaccccaaggtggggagaaccaacaaagatgtgtcgcgcatgagatctataatcctacagctgaagcagactccgct

SEQ ID NO: 81 - DNA, 273 bp, Bactericera cockerelli
acgggtcatgctctagctctctaccgaaatgatgaatatattagtgatcttgccattcatactataaaaagaatcaagctttacagtgactcactagcacgctatggcaagtctccatacttgtaccccatgtatggactgggagaactgcctcaaagttttgcccgtctaagtgcaatctatggtggaacctacatgttggataaacctgtagatgaaatagtgtttgagaatggtaaagtagttggtgtacgatctggcagtgaaacagct

SEQ ID NO: 82 - DNA, 156 bp, Bactericera cockerelli
tctgaacttgacgacagagcggatgcacttcaacaaggagcttcacagtttgaacagcaggcgggaaaactgaaaaggaagttttggctgcaaaatttaaaaatgatgattattatgggtgttattggcctagtcatagttgccatcatagtgggt

SEQ ID NO: 83 - DNA, 200 bp, Bactericera cockerelli
ttcgatttcgaccgattttccaagcacgatcaaatcggggaggtaaaagtggctctttgccagatcgatctggcccaaaccattgaagagtggcgggaactgcagagtgtagaaggagaaggaggacaggataacaagttgggagacatctgcttctctctgcgctacgtccccaccgctggaaaactcaccgtggtgat

SEQ ID NO: 84 - DNA, 235 bp, Bactericera cockerelli
ttgggtagattcctcgccttgaggatgacgatggtgaagcggttagcggccggctgccagcagagagacagcaggatctcaccccggccctgcgactttattcgcaggctgcggggctggatgtcgcgacagaaggagagggagtggccggtggtctcgaaggactgcagagagtagaagacctcgcccacaatgtcgtcccgggagtagcggtcgaagctgaacaccacgaagt

SEQ ID NO: 85 - DNA, 212 bp, Bactericera cockerelli
aaggacctgtacgccaacactgtcctgtccggtggtaccaccatgtaccccggtatcgccgacagaatgcagaaggaaatcactgccctggctccttccaccatcaagatcaagatcatcgctccccccgagagaaagtactccgtatggatcggtggttccatcctggcctctctgtccaccttccagcagatgtggatctccaaacagga

SEQ ID NO: 86 - DNA, 246 bp, Bactericera cockerelli
agaatctgggacactgtgaacaaggagcatattctaaagaatgaatttcatcctattggaggacctatcaaagatattgcttggtcacctgataaccagcgtatggttgttgtgggtgaaggaagagaaagatttggccatgtttttatggcagaaactggtacatcagtgggagaaatttctggccagtcaaagcccatcaactcttgtgatttcaaaccttctcgtcctttccgagtgatcact

SEQ ID NO: 87 - DNA, 225 bp, Bactericera cockerelli
atctcggagatcgtggacacggtgattcagaactgcccgatagacgtgcgtcgcccgctctaccacaacatagtgctcagtggaggctccaccatgttccgggactttgggcggaggctgcagagagatatcaagcgagtggtggacgcgaggctgaaactgagcgaaaccttgagtggtggatacattaagcccacccccatagacgtgcaggtgatcacacac

SEQ ID NO: 88 - DNA, 209 bp, Bactericera cockerelli
ttgtaaccagcgtggctacagctgggggagtggtttgctttcactttgcttccgaaatacttggtctggaccaaagttattactgcagtggccgatgtgctatcttgaattcagggcagagttgttctgaacatggcagtgacttcatcatccgagagcttcgagaacaatcattctctcctgtggaacgttctccccgtgattgtctt

SEQ ID NO: 89 - DNA, 263 bp, Bactericera cockerelli
aaccattgaccaggctacaagagcggctaatgaagaagctgggtccgaacgcttttccttttttcttcgagctacctccgtcctgccccgcctcagtcacactacagcccgcccctggggacacaggcaaaccctgcggagttgactacgaactgaaagcttttgtgggagatacagctgaagataaaatacacaagagaaattcagtgaggctagcaataagaaaaatcatgtatgctccaagtaagcaaggagaacagcct

SEQ ID NO: 90 - DNA, 162 bp, Bactericera cockerelli
ttcaggcgtaacggtgtccgacatttgcaagaccacctacgaggagatcaagaaggacaagaagcaccgctacgtggtgttcttcattcgcgacgagaagcagatcgacgtggagtacattggcgaccgcaacgccacctacgactcgttcctggaggatct

SEQ ID NO: 91 - DNA, 278 bp, Bactericera cockerelli
agagttcctggcctcaagatctggagaatagagaaattcgaaccagtgcccgtaccagaaaagagctatggcaaattctactccggtgattcatacattgttttaaataccaaggaagaaaagggaaacaagaagaaaaccttctcatacgacatccactactggctgggcaaggaaacttctcaggatgaatctggagcggccgctatcctgactgtggacctggacgatagtctggggggaggtcctgtgcagcacagggaagtggaggaacatga

SEQ ID NO: 92 - DNA, 192 bp, Bactericera cockerelli
agagctgagcaaaatgaagcaggaacccgtgaagcctgaggagggccgtgccatggctcaaaaaatcaatgcatttgcctacttggagtgttccgccaaaagtaaagaaggtgtgcgtgaagtgtttgaaacagcaacacgagctgctcttcaagttaaaaagaagaagaagggccgctgtagactcttgta

SEQ ID NO: 93 - DNA, 194 bp, Bactericera cockerelli
aatcaagtgtgtagttgtgggagatggagctgtgggtaaaacttgtctgcttatttcatacacaacaaacaaattcccctcagagtatgttcccactgtgtttgacaattatgctgtcactgtcatgattggaggagaaccatacactttaggtctgtttgacacagcgggtcaggaagactatgacaggctgc

SEQ ID NO: 94 - DNA, 244 bp, Bactericera cockerelli
aacaagaaactggccgaagctctggtggcggccgcaggacaggtggagtccctgacgccgcagctcgtgaatgcgggccgcatccgcatgacctacccgcagagcaaggccgcagacgagcactttgagaatctgcggaaccagtatgcgggctctgtgggtcggctgcgggacctgtgtgacgagaccattgacccggccgagtttgtcaagtactcggaggagcaaatgaagaagcacacca

SEQ ID NO: 95 - DNA, 229 bp, Bactericera cockerelli
agacggccctaacgataacgagtggcacaagaggtgtttcggcattctgtgccttgtcaaggataatccccgcaaatcctatttcttccgactctactgtttaacgaggagacaactggtttgggaacatgagctctataaaggcatgagctacatggccccacagaacttcctgcacacattcgaagccgaggattgtatcgtggccttcaactttgccaatgaggaa

SEQ ID NO: 96 - DNA, 142 bp, Bactericera cockerelli
tctgcccaagcgagtgatagaacctgtccacgtagcccgaggcaccatccctgatgagctgtccgctagtctaccttcggaacttgaggccgccaccaatggaaccctggctaacacggttcgtcaactgtccagcctcagt

SEQ ID NO: 97 - DNA, 277 bp, Bactericera cockerelli
aggttctgcaatagcaccagttgttccggacgaagatgatgactgggaaactgatcctgacttcatcaatgatgtaagtgaacaggagcagaggtggggctccaaaactataccaggttctggtagagatgctgggtctattgacatgaagcaactgcgggaagaagtggctatgtcagatgcatgctacaagcaaaagcagttagatggaggatcaaaagcttcttttggatatggaggaaaatttggtgttgagaaggataggatggatcagtca

SEQ ID NO: 98 - DNA, 272 bp, Bactericera cockerelli
agtactgcagggacaagagccaggaccatggcaagaaagtgaaccatgttcaaagtcaggttcgagcctggctggcaggggggcagacctcccccatgtgcaccgccagggctgggtggaagagcatgaccctgcgggagcccaagtacaaggcgaccatgttccctgtggacctgggacctctggattccatcctctctgttgacgaacacagtcatacggttctcgttgagccttacgtgaccatgggtcagctgacacgctacctcatt

SEQ ID NO: 99 - DNA, 182 bp, Bactericera cockerelli
atggaatcaagcgagctgcgacctcgggacgcgaaatacgtttcggcgctcggggagaatttcgccgtgttccgatcggaatcgggcgaggtgcatgtgctggatgcctactgcccgcatttgggcgccaacatggcgatcggtgggttcgtgcggggggactgcatcgagtgtcccttcca

SEQ ID NO: 100 - DNA, 277 bp, Bactericera cockerelli
ttctgaagtggccatgcagttgggggtcctggaattaaggcatttcctcaaggatatgccccatgcctcgttggataaaaagtcgactcttgattatttagaacgagaagttggatttcacaagttcctaccaaagcacatagtccaaacttcaaagccaaagaccctccgcaaaacacttcagtcccattttaagaaaatcgctcatctctcagagaaagattgcataatgaggttcttcgagatcctcagatctcattacaagtttgaccaggaa

SEQ ID NO: 101 - DNA, 233 bp, Bactericera cockerelli
ttccaaatctgtttctcgctcgtgaacccggcctcgttcgagaacgtgcgagccaagtggtaccccgaggtgcggcaccactgccccaacacgcccatcattctggtgggcaccaaactggacctgcgggacgacaaggagaccattgagaagctcaaggagaagaaactggcgcccatcacatacccccagggcctgtccatggcgaaggagatcggggccgtcaagtatct

SEQ ID NO: 102 - DNA, 208 bp, Bactericera cockerelli
caaatgtgatttaagcacttgggctgttgacatgaatcaagcacaagaacttgcagaacaattcaacattcccttcatcaaaacttcagccaaaacacgtatgggagttgatgatgctttctacacacttgtccgagaaatcaagaaagataagatgctccgaggtaaagaaaagaagaagcgaggaatcagtggaaacaaactgaag

SEQ ID NO: 103 - DNA, 191 bp, Bactericera cockerelli
tgatggacgcaccataaaagttggaggtgaaagatttgaagccccggaaattctgttccaacctcacctaatcaatgtcgaaggacaaggcattgctgagttggtgttcaacacgatacaggctgccgacatcgacgtgaggactcagctatacaagcacatcgtcctgtctggaggctccaccatgtatc

SEQ ID NO: 104 - DNA, 191 bp, Bactericera cockerelli
tcgagcgatcaaaccgtacgcccgcaaagcggccgtgcccgtggaagacaagaaactgcgcaagaaagagcagaacaagaacgcggccacgcgctatcgaatcaagaagaaggcggagatcgaagtcattctgggcgaggagtcggaactcagggagaagaacgaggagctgcagaagagtgtggaggatt

SEQ ID NO: 105 - DNA, 200 bp, Bactericera cockerelli
tgcgcagactcaaacgttccaagatccgcatcatcctgggtgactacgatcagttctcgaccacggaaacacccgaaccaacacaaatgcgagctgtctcttcaatcgtgcgtcatcggcacttcgacgtgaacagctacaaccacgacatcgccttgctcaagctacgcaagcccgtggcattcagcaagagtgtgcgt

SEQ ID NO: 106 - DNA, 153 bp, Diaphorina citri
ttatctcagagcatttacatccccaagggtgtcaacattcctgcattgaacagagatgttagttgggagttcaacccaatgaatctgaagattggaagtcatatcactggtggagatcagtatggtcttgttcatgagaatacacttgtcaag

SEQ ID NO: 107 - DNA, 130 bp, Diaphorina citri
tcgataagctcgaagcaagctttacgggagaatgtgctagctgtgacgcgggactacatatcgcagccacgaataacatacaaaactgtgtctggtgtcaacggacctctggtcatcttagacgaggtga

SEQ ID NO: 108 - DNA, 140 bp, Diaphorina citri
tctcgtctgaagggagctcagaagggacacagtcttctcaagaagaaggctgatgctctacagatgaggttccgaatgattctcagcaagattattgagactaaaactctcatgggagaggtaatgaaagaagctgcgtt

SEQ ID NO: 109 - DNA, 149 bp, Diaphorina citri
aagcaaatcgaacgcatggtggcgtttatccaaaccgaggcggacgaaaagttggacgacatccggcgaaaaatcgaagaggactaccagatcgaaagagagcgagtaacgcgagacggaaaggcgagcgtggatgaagaatatgctaa

SEQ ID NO: 110 - DNA, 142 bp, Diaphorina citri
acagaggatcgcacaaagtacaccgagatactcgaaaaactcattttccaaggtgtcctcaagttactggaacccactgttctgattcgatgtaaagaaaacgacctgtgcattgtgaggcaactactgccgctagtagcta

SEQ ID NO: 111 - DNA, 154 bp, Diaphorina citri
tgggtggaagtgtatgtcactgagagagcccaagtacaagtcgtccatgtttccagtggatctggaggcgatggataccatcctgagtgtggacgaggagaagaagacggtcaaagtagagccctatgtgaccatgggtcaattaacccgctat

SEQ ID NO: 112 - DNA, 151 bp, Diaphorina citri
acggacactcaatacaatcccctctcacgaatcctccgatggcaatgttagctcccaaatgaggacagtacgcatccagcacatgcacttctcccctctccgatcggaacacagcaaagttttccccgagtgcggaaacatatttggcctg

SEQ ID NO: 113 - DNA, 157 bp, Diaphorina citri
tacctattgaagacaagcgcctgcgcaagaaggagcagaataagaacgcggcgacgcgttataggattaagaagaaggcggagattgaggaggttctgggagaggagaaagagctgttggagaagaacgcccagctgcagaagagtgtggaagatct

(19) United States
(12) Patent Application Publication
(10) Pub. No.: US 2024/0043864 A1
(43) Pub. Date: Feb. 8, 2024

(54) COMPOSITIONS AND METHODS FOR CONTROLLING PSYLLIDS

(71) Applicant: Arizona Board of Regents on Behalf of the University of Arizona, Tucson, AZ (US)

(72) Inventors: Judith K. Brown, Tucson, AZ (US); Mosharrof Hossain Mondal, Tucson, AZ (US); Jorge Rafael Paredes Montero, Tucson, AZ (US)

(21) Appl. No.: 18/269,021

(22) PCT Filed: Dec. 22, 2021
(86) PCT No.: PCT/US2021/064845

§ 371 Date: Jun. 22, 2023

Related U.S. Application Data
(60) Provisional application No. 63/130,152, filed on Dec. 23, 2020.

(51) Int. Cl. C12N 15/82; C12N 15/113

(52) U.S. Cl. CPC C12N 15/8286; C12N 15/113; C12N 2310/14

(57) ABSTRACT

The present invention relates to compositions and methods for controlling psyllid infestation of plants. In particular, the present invention provides vectors comprising sequences designed to control psyllids by RNA interference (RNAi) and transgenic plants transformed with such vectors.

### CROSS-REFERENCE TO RELATED APPLICATIONS

The present application claims priority to U.S. Provisional Patent Application No. 63/130,152, filed Dec. 23, 2020, which is hereby incorporated by reference in its entirety.

### STATEMENT REGARDING FEDERALLY SPONSORED RESEARCH OR DEVELOPMENT

This invention was made with government support under Grant No. 2012-51181-20086, awarded by USDA/NIFA. The government has certain rights in the invention.

### FIELD OF THE INVENTION

The present invention relates to compositions and methods for controlling psyllid infestations of plants and infection of plants by Ca. Liberibacter pathogen inoculation by the psyllid vector(s). In particular, the present invention provides dsRNA molecules delivered and/or dsRNA molecules cloned into plasmid vectors comprising sequences designed to control psyllids by RNA interference (RNAi) delivered by non-transgenic routes, and by expression in transgenic plants transformed with such plasmid vectors/constructs.

### BACKGROUND OF THE INVENTION

RNA interference (RNAi) is a potentially powerful gene-silencing tool for analysis and knockdown of gene function. The mechanism of RNAi in animals was first identified in the free-living nematode Caenorhabditis elegans, in which the expression of unc22 gene was suppressed via the RNAi pathway (Fire et al. 1998). During this process, long double-stranded RNA is processed into 21-23 nucleotide siRNAs by Dicer, a member of the RNase family (Bernstein et al. 2001). The DCR-2/R2D2 complex binds to siRNAs and enhances sequence-specific messenger RNA degradation mediated by the RNA-initiated silencing complex (Liu et al. 2003). This pathway recently has shown promise as the basis of a novel control strategy for plant-parasitic nematodes, with numerous independent studies demonstrating suppression of target nematode populations following soaking nematodes in dsRNA solutions (Urwin et al. 2002; Bakhetia et al. 2005; Huang et al. 2006; Alkharouf et al. 2007) and, more importantly, using in planta transgenic systems expressing dsRNA fragments of nematode genes (Huang et al. 2006; Steeves et al. 2006; Yadav et al. 2006; Sindhu et al. 2009). Yadav et al. (2006) reported that RNAi was induced by using dsRNA fragments of two genes encoding an integrase and a splicing factor in the plant-parasitic nematode M. incognita, leading to protection against nematode infection in tobacco. The expression of root-knot nematode parasitism gene 16D10 dsRNA in transgenic Arabidopsis resulted in resistance against four major root knot nematode species (Huang et al. 2006), while Sindhu et al. (2009) obtained reductions in H. schachtii females ranging from 23 to 64% in transgenic Arabidopsis lines expressing RNAi constructs of four parasitism genes. Bioassay data indicated transgenic plants had up to a 68% reduction in eggs g⁻¹ root tissue. The effects of plant-derived dsRNA molecules appeared to continue into the next generation.

The most critical obstacle confronting the US citrus industry is the inability to control the citrus greening disease, caused by Ca. Liberibacter asiaticus (CLas), which spread rapidly through Florida beginning in 2006, after the 2002 introduction of the Asian citrus psyllid (ACP; Diaphorina citri) vector. Since the establishment of ACP in the US, it has ravaged the Florida (FL) citrus industry, spread into Texas (TX) and other southern U.S. states, and has been recently been identified in California (CA). As a result, for AZ, the ACP populations dispersing from Mexico and CA to Arizona (AZ) pose a threat to commercial lemons and other citrus varieties, HLB-free nursery program sustainability, and urban citrus trees, despite high vigilance and quarantine measures. Further, Ca. Liberibacter solanacearum (CLso) is a recently emergent, economically-important bacterial pathogen of solanaceous crops, including eggplant, pepper, tomatillo, and tomato (green-veining disease) (Brown et al., 2010) and potato (zebra chip disease) of importance in the U.S. and elsewhere in the American Tropics where it is endemic, and in other locales where it has been accidentally introduced. Other Ca. Liberibacter spp/variants infect carrot, celery, and other crop plants, resulting in crop loss. In all known instances, a psyllid vector transmits the fastidious Liberibacter (bacterial) pathogen. See the world wide web at onlinelibrary.wiley.com/doi/10.1111/epp.12043/.

Novel approaches for psyllid vector management are needed to protect susceptible plants of economic importance from psyllid infestations and Liberibacter infection, to abate two of the most dire diseases, zebra chip of potato and citrus greening.

### SUMMARY OF THE INVENTION

The present invention relates to compositions and methods for controlling psyllid infestation of plants and abating Ca. Liberibacter transmission to plants. In particular, the present invention provides dsRNA molecules and cloned dsRNA(s) (in plasmid vectors for delivery by transgenic or other means, i.e., injection, topical application, phloem, foliar or root uptake) comprising sequences designed to control psyllids by non-transgenic RNA interference (RNAi) and transgenic plants transformed with such plasmid constructs.

In some preferred embodiments, the present invention provides double-stranded ribonucleic acid (dsRNA) comprising a sense region with at least 80% sequence identity to a sequence comprising at least 15 consecutive nucleotides up to the entire length of an RNA sequence encoded by a sequence selected from the group consisting of SEQ ID NOs: 1-113, and an antisense region comprising a second sequence complementary entirely to the sense region.

In some preferred embodiments, the dsRNA comprises a sense region with at least 90% sequence identity to a sequence comprising at least 15 consecutive nucleotides up to the entire length of an RNA sequence encoded by a sequence selected from the group consisting of SEQ ID NOs: 1-113, and an antisense region comprising a second sequence complementary entirely to the sense region. In some preferred embodiments, the dsRNA comprises a sense region with at least 95% sequence identity to a sequence comprising at least 15 consecutive nucleotides up to the entire length of an RNA sequence encoded by a sequence selected from the group consisting of SEQ ID NOs: 1-113, and an antisense region comprising a second sequence complementary entirely to the sense region. In some preferred embodiments, the dsRNA comprises a sense region with at least 99% sequence identity to a sequence comprising at least 15 consecutive nucleotides up to the entire length of an RNA sequence encoded by a sequence selected from the group consisting of SEQ ID NOs: 1-113, and an antisense region comprising a second sequence complementary entirely to the sense region. In some preferred embodiments, the dsRNA comprises a sense region with 100% sequence identity to a sequence comprising at least 15 consecutive nucleotides up to the entire length of an RNA sequence encoded by a sequence selected from the group consisting of SEQ ID NOs: 1-113, and an antisense region comprising a second sequence complementary entirely to the sense region. In some preferred embodiments, the dsRNA sequence is encoded by a selected from the group consisting of SEQ ID NOs:1 to 49. In some preferred embodiments, the dsRNA sequence is encoded by a selected from the group consisting of SEQ ID NOs:50 to 56. In some preferred embodiments, the dsRNA sequence is encoded by a selected from the group consisting of SEQ ID NOs:57 to 113. In some preferred embodiments, the dsRNA sequence is encoded by a selected from the group consisting of SEQ ID NOs:57 to 105. In some preferred embodiments, the dsRNA sequence is encoded by a selected from the group consisting of SEQ ID NOs:106 to 113. In some preferred embodiments, the sense region comprises at least 21 consecutive nucleotides up to the entire length of a sequence selected from the group consisting of SEQ ID NOs: 1-113.

In some preferred embodiments, the present invention provides a plant cell comprising a dsRNA sequence as described above. In some preferred embodiments, the plant cell is a tree cell.

In some preferred embodiments, the present invention provides a transgenic plant, transgenic plant cell, or transgenic seed comprising a dsRNA sequence as described above.

In some preferred embodiments, the present invention provides a bacterial or yeast host cell comprising a dsRNA sequence as described above.

In some preferred embodiments, the present invention provides a DNA molecule comprising a promoter functional in a host cell and a DNA encoding a dsRNA comprising a first region and a second region, wherein the first region comprises a sense region with at least 80% sequence identity to a sequence comprising at least 15 consecutive nucleotides up to the entire length of a sequence selected from the group consisting of SEQ ID NOs: 1-113, and a second region complementary entirely to the sense region. In some preferred embodiments, the host cell is a bacterial cell, a yeast cell or a plant cell.

In some preferred embodiments, the present invention provides a host cell comprising the DNA molecule described in the preceding paragraph. In some preferred embodiments, the host cell is a plant cell. In some preferred embodiments, the plant cell is a tree cell. In some preferred embodiments, the present invention provides a transgenic plant cell, transgenic plant or transgenic seed comprising the DNA molecule as in the preceding paragraph.

In some preferred embodiments, the present invention provides a DNA molecule comprising convergent promoters functional in a host cell flanking a DNA segment with at least 80% sequence identity to a sequence comprising at least 15 consecutive nucleotides up to the entire length of a sequence selected from the group consisting of SEQ ID NOs: 1-113. In some preferred embodiments, upon expression of the DNA molecule in a host cell a dsRNA is produced. In some preferred embodiments, the host cell is a bacterial cell, a yeast cell or a plant cell.

In some preferred embodiments, the present invention provides a host cell comprising the DNA molecule described in the preceding paragraph. In some preferred embodiments, the host cell is a plant cell. In some preferred embodiments, the plant cell is a tree cell. In some preferred embodiments, the present invention provides a transgenic plant cell, transgenic plant or transgenic seed comprising the DNA molecule described in the preceding paragraph.

In some preferred embodiments, the present invention provides a method of controlling psyllids comprising, planting or growing a transgenic plant expressing a dsRNA as described above and allowing one or more psyllids to ingest an effective amount of the dsRNA, thereby controlling the one or more psyllids, and/or interfering with Ca. Liberibacter transmission that results in abatement by any mode of interference resulting from dsRNA activity. In some preferred embodiments, the psyllids are Bactericera cockerelli. In some preferred embodiments, the psyllids are Diaphorina citri.

In some preferred embodiments, the present invention provides a method of controlling psyllids comprising applying the dsRNA of any one of claims 1 to 11 to a plant on which one or more psyllids feed and allowing the one or more psyllids to ingest an effective amount of the dsRNA, thereby controlling the one or more psyllids. In some preferred embodiments, the dsRNA is present in a transgenic bacterial cell.

In some preferred embodiments, the present invention provides a method of controlling citrus greening disease in citrus plants comprising planting or growing a transgenic citrus plant expressing a dsRNA as described above and allowing one or more psyllids of the species Diaphorina citri to ingest an effective amount of the dsRNA, thereby controlling the one or more psyllids and spread of Ca. Liberibacter asiaticus.

In some preferred embodiments, the present invention provides a method of controlling citrus greening disease in citrus plants comprising applying a dsRNA as described above to a citrus plant on which one or more psyllids of the species Diaphorina citri feed and allowing the one or more psyllids to ingest an effective amount of the dsRNA, thereby controlling the one or more psyllids and spread of Ca. Liberibacter asiaticus. In some preferred embodiments, the dsRNA is present in a transgenic bacterial cell and/or is delivered as a topically applied composition or is delivered via a viral vector.

In some preferred embodiments, the present invention provides a method of controlling disease in plants comprising planting or growing a transgenic plant expressing a dsRNA as described above and allowing one or more psyllids of the species Bactericera cockerelli to ingest an effective amount of the dsRNA, thereby controlling the one or more psyllids and spread of Ca. Liberibacter solanaceearum.

In some preferred embodiments, the present invention provides a method of controlling citrus greening disease in citrus plants comprising applying a dsRNA as described above to a citrus plant on which one or more psyllids of the species Bactericera cockerelli feed and allowing the one or more psyllids to ingest an effective amount of the dsRNA, thereby controlling the one or more psyllids and spread of Ca. Liberibacter solanaceearum. In some preferred embodiments, the dsRNA is present in a transgenic bacterial cell and/or is delivered as a topically applied composition or is delivered via a viral vector.

### Definitions

To facilitate an understanding of the present invention, a number of terms and phrases as used herein are defined below:

The term “plant” is used in it broadest sense. It includes, but is not limited to, any species of woody, ornamental or decorative, crop or cereal, fruit or vegetable plant, and photosynthetic green algae. It also refers to a plurality of plant cells that are largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

The term “crop” or “crop plant” is used in its broadest sense. The term includes, but is not limited to, any species of plant or algae edible by humans or used as a feed for animals or used, or consumed by humans, or any plant or algae used in industry or commerce.

The term plant cell “compartments or organelles” is used in its broadest sense. The term includes but is not limited to, the endoplasmic reticulum, Golgi apparatus, trans Golgi network, plastids including chloroplasts, proplastids, and leucoplasts, sarcoplasmic reticulum, glyoxysomes, mitochondrial, chloroplast, and nuclear membranes, and the like.

The term “host cell” refers to any cell capable of replicating and/or transcribing and/or translating a heterologous gene.

The term “heterologous,” when used in reference to DNA sequences or genes, means a DNA sequence encoding a protein, polypeptide, RNA, or a portion of any thereof, whose exact amino acid sequence is not normally found in the host cell, but is introduced by standard gene transfer techniques.

As used herein, “dsRNA” refers to double-stranded RNA that comprises a sense and an antisense portion of a selected target gene (or sequences with high sequence identity thereto so that gene silencing can occur), as well as any smaller double-stranded RNAs formed therefrom by RNAse or dicer activity. Such dsRNA can include portions of single-stranded RNA, but preferably contain at least 19 nucleotides double-stranded RNA. In some embodiments of the invention, a dsRNA comprises a hairpin RNA which contains a loop or spacer sequence between the sense and antisense sequences of the gene targeted, while in other embodiments of the invention the dsRNA is produced by expression from convergent promoters.

The term “RNA interference” or “RNAi” refers to the silencing or decreasing gene expression by miRNAs or siRNAs, or piRNAs. It is the process of sequence-specific, posttranscriptional and transcriptional gene silencing in animals and plants, initiated by iRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “interfering RNA (iRNA)” refers to a double-stranded RNA molecule that mediates RNA interference (RNAi). At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the RNAi antisense strand. RNAi may also contain additional sequences; non-limiting examples of such sequences include linking sequences, or loops, as well as stem and other folded structures.

siRNAs generally comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA or DNA (including endogenous elements) genomes.

As used herein, the term “loop sequence” refers to a nucleic acid sequence that is placed between two nucleic sequences that are complementary (to each other) and which forms a loops when the complementary nucleic acid sequences anneal or base pair to one another.

“Insecticidal activity” of a dsRNA, as used herein, refers to the capacity to obtain mortality in insects when such dsRNA is fed to insects, preferably by expression in a recombinant host such as a plant, which mortality is significantly higher than a negative control (using a non-insect dsRNA or buffer). “Insect-control” of a dsRNA, as used herein, refers to the capacity to inhibit the insect development, fertility, inhibition of pheromone production, or growth in such a manner that the insect population provides less damage to a plant, produces fewer offspring, are less fit or are more susceptible to predator attack, or that insects are even deterred from feeding on such plant.

The term “psyllid target RNA” as used herein refers to a coding or non-coding RNA that is expressed in a psyllid.

The term “double stranded psyllid RNA sequence” refers to an iRNA that is specific for a psyllid target RNA.

The term “inhibits the proliferation of psyllids” refers to a reduction in psyllid parasitism of a host organism. A variety of assays may be used to measure proliferation.

As used herein, the term “orally active to prevent the proliferation of psyllids” refers to a double stranded psyllid RNA sequence that inhibits the proliferation of psyllids when orally ingested by the psyllids.

The terms “protein” and “polypeptide” refer to compounds comprising amino acids joined via peptide bonds and are used interchangeably.

As used herein, “amino acid sequence” refers to an amino acid sequence of a protein molecule. “Amino acid sequence” and like terms, such as “polypeptide” or “protein,” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′- and 3′-end for a distance of about 1 kbp on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′- and 3′-end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation. Other non-coding sequences may also be present, including, but not limited to piRNAs. PIWI-interacting RNAs (piRNAs) are single-stranded, 23-36 nucleotide (nt) RNAs that act as guides for an animal-specific class of Argonaute proteins, the PIWI proteins. The first piRNAs were derived from the Suppressor of Stellate locus in Drosophila melanogaster testes in 2001.

The term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant or animal gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous genes are distinguished from endogenous plant or animal genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that rely on interactions such as base pairing between nucleic acids.

The term “homology” when used in relation to nucleic acids refers to a degree of complementarity. There may be partial homology or complete homology (i.e., identity). “Sequence identity” refers to a measure of relatedness between two or more nucleic acids, and is given as a percentage with reference to the total comparison length. The identity calculation takes into account those nucleotide residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of base pairing of the completely complementary sequence to the target sequence may be examined using a hybridization assay (Southern or Northern blot, solution hybridization, and analogous methods) under conditions of low stringency. A substantially homologous sequence or probe will compete for and inhibit the binding (i.e., the hybridization) of a sequence that is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target. In a preferred embodiment, a homolog has a greater than 60% sequence identity, and more preferable greater than 75% sequence identity, and still more preferably greater than 90% sequence identity, with a reference sequence.

When used in reference to a double-stranded nucleic acid sequence such as a cDNA or genomic clone, the term “substantially homologous” refers to any oligonucleotide or other probe which can base pair to either or both strands of the double-stranded nucleic acid sequence under conditions of low stringency as described infra.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue (e.g., seed tissue) in the relative absence of expression of the same nucleotide sequence of interest in a different type of tissue (e.g., leave tissue). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Exemplary constitutive plant promoters include, but are not limited to SD Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098), and ubi3 (see e.g., Garbarino and Belknap (1994) Plant Mol. Biol. 24:119-127) promoters. Such promoters have been used successfully to direct the expression of heterologous (non-self) nucleic acid sequences in transformed plant cells, tissues, and/or organs.

In contrast, a “regulatable” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

The term “vector” when used in relation to a nucleic acid construct refers to nucleic acid molecules that transfer DNA segment(s) from one cell to another. The term “vehicle” is sometimes used interchangeably with “vector.”

The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “transfection”, “transformation”, “transfected” and “transformed” are used interchangeably and refer to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (e.g., nopaline, agropine, octopine etc.) by the infected cell. Thus, Agrobacterium strains which induce production of nopaline (e.g., strain LBA4301, C58, A208, GV3101) are referred to as “nopaline-type” Agrobacteria; Agrobacterium strains which cause production of octopine (e.g., strain LBA4404, Achy, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “microwounding” when made in reference to plant tissue refers to the introduction of microscopic wounds in that tissue. Microwounding may be achieved by, for example, particle bombardment as described herein.

The term “transgenic” when used in reference to a plant or fruit or seed (i.e., a “transgenic plant” or “transgenic fruit” or a “transgenic seed”) refers to a plant or fruit or seed that contains at least one heterologous gene in one or more of its cells. The term “transgenic plant material” refers broadly to a plant, a plant structure, a plant tissue, a plant seed or a plant cell that contains at least one heterologous gene in one or more of its cells.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “overexpression” refers to the production of a gene product in transgenic organisms that exceeds levels of production in normal or non-transformed organisms. The term “cosuppression” refers to the expression of a foreign gene that has substantial homology to an endogenous gene resulting in the suppression of expression of both the foreign and the endogenous gene. The term “altered levels” refers to the production of gene product(s) in transgenic organisms in amounts or proportions that differ from that of normal or non-transformed organisms.

The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature.

The term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. The term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

### DESCRIPTION OF THE INVENTION

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; and Kaufman et al., eds., “Handbook of Molecular and Cellular Methods in Biology and Medicine”, CRC Press, Boca Raton, 1995.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

The present invention relates to compositions and methods for controlling psyllid infestation of plants. In some particularly preferred embodiments, the present invention provides methods of controlling citrus greening disease in citrus plants (e.g., citrus trees). Citrus greening disease is caused by Ca. Liberibacter asiaticus (CLas) which is spread by the Asian citrus psyllid (ACP) vector (Diaphorina citri). It is contemplated that by controlling the vector, infection of citrus plants by CLas can be controlled or reduced. Accordingly, the present invention provides vectors comprising sequences designed to control psyllids by RNA interference (RNAi) and transgenic plants transformed with such vectors. The compositions and methods of the present invention can be used to inhibit the growth and reproduction of a number of psyllid species, including, but not limited to Diaphorina citri (Asian citrus psyllid, ACP) and Bactericera cockerelli (potato psyllid, PoP). In other preferred embodiment, the present invention contemplates transmission abatement or interference with ‘transmission’ by knockdown of psyllid genes encoding proteins required by Liberibacter for invasion, multiplication, exocytosis, translocation in blood, or entry into the salivary glands, after which the psyllid vector becomes Liberibacter-transmission competent.

The present inventors have conducted rigorous in silico mining of transcriptome and proteome databases, and gene identifications based on the KEGG pathway and network databases, and carried out in vitro protein-protein interactions screens using yeast-2-hybrid and co-immunoprecipitation to inform biologically interesting targets for the control of psyllids. The most promising have been advanced to in vivo dsRNA knockdown, based on oral ingestion in sucrose feeding chambers and in planta assays (UV laser delivery; cut-stem uptake; root uptake; phloem injection delivery), validated by qPCR, and mortality or transmission bioassays in a tomato PoP model for the spread of Ca. Liberibacter solanacearum (CLso). Advantages of the PoP-CLso ‘fast-track study system’ are ease of PoP rearing, high CLso titer in rapidly growing tomato plants and in psyllids reared on them, symptom development in 10-20 d, and qPCR detection of CLso in newly developing leaves within several days, compared to ACP-CLas, which can require weeks to months. Conveniently, PoP and ACP share >60% homologous contigs, (Brown et al., unpubl.), making it straightforward to locate PoP and ACP homologs in transcriptome libraries for dsRNA design. In this way, we can rapidly screen CLso/CLas effector interactors (˜60 tested) and eliminate those with low knockdown potential, moving top-ranking candidates to ACP-CLas screening.

RNAi offers a highly target-specific, non-toxic, biopesticide solution for reducing ACP population size and suppressing transmission efficiency, to seedlings and older uninfected. Studies have shown dsRNA is safe to use, and that dsRNA exposed to environmental degradation loses activity in 2d or less. Thus far, there have been no negative documented consequences to the ‘greater environment, post-dsRNA exposure’.

The present invention provides dsRNA targets identified and tested to varying extents for knockdown and phenotype (mortality, development, fecundity, transmission interference) in the potato psyllid (PoP), and homologous targets for selected Asian citrus psyllid genes, mined from the ACP genome sequence version 3.

### I. RNAi Systems, Constructs and Vectors

RNAi refers to the introduction of homologous double stranded RNA (dsRNA) to target a specific gene product, resulting in post transcriptional silencing (PTS) of that gene. This phenomena was first reported in Caenorhabditis elegans by Guo and Kemphues (Par-1, A gene required for establishing polarity in C. elegans embryos, encodes a putative Ser/Thr kinase that is asymmetrically distributed, 1995, Cell, 81 (4) 611-620) and subsequently Fire et al. (Potent and specific genetic interference by double-stranded RNA in Caenorhabditis elegans, 1998, Nature 391: 806-811) discovered that it is the presence of dsRNA, formed from the base-pairing/annealing of sense and antisense strands present in the in vitro RNA preps, that is responsible for producing the interfering activity. The present invention contemplates the use of RNA interference (RNAi) to downregulate the expression of genes needed for pest viability and reproduction, thus reducing pest infestation of plants. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs.

Carthew has reported (Curr. Opin. Cell Biol. 13(2):244-248 (2001) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

Silencing RNA can be derived from exogenous or intracellular origins, depending on the organism and cell type. RNA can also be introduced artificially using siRNA or plasmid-based short hairpin RNA (shRNA) systems. RNAs transcribed from the genome may be retained in the nucleus (as with piRNAs) to carry out silencing or may be exported (as with miRNAs). In the cytoplasm, dsRNA is processed by the endonuclease Dicer and loaded onto an Argonaute protein, and after the strand selection process, the newly formed RISC is equipped to silence target genes by one of several mechanisms. Although the mechanisms used to control gene expression by RISC are quite diverse, two central themes are common to all. First, at its core, every RISC contains a member of the Argonaute protein family that binds to the small regulatory RNA. Second, in every RISC, the small regulatory RNA functions as a guide that leads RISC to its target through Watson-Crick base pairing with cognate RNA transcripts. The role of the Argonaute protein is to bind the small RNA and position it in a conformation that facilitates target recognition. Argonaute proteins can either cleave target RNAs directly or recruit other gene-silencing proteins to identified targets. Here, we review how Argonaute proteins use small RNAs to recognize target transcripts. We also examine how recruitment of different types of Argonaute and Argonaute-associated proteins produce distinct RISCs, which then dictate the mechanism of gene regulation.

In preferred embodiments, the dsRNA used to initiate RNAi, may be isolated from native source or produced by known means, e.g., transcribed from DNA. The promoters and vectors described in more detail below are suitable for producing dsRNA. RNA is synthesized either in vivo or in vitro. In some embodiments, endogenous RNA polymerase of the cell may mediate transcription in vivo, or cloned RNA polymerase can be used for transcription in vivo or in vitro. In other embodiments, the RNA is provided transcription from a transgene in vivo or an expression construct. In some embodiments, the RNA strands are polyadenylated; in other embodiments, the RNA strands are capable of being translated into a polypeptide by a cell's translational apparatus. In still other embodiments, the RNA is chemically or enzymatically synthesized by manual or automated reactions. In further embodiments, the RNA is synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. In some embodiments, the RNA is dried for storage or dissolved in an aqueous solution. In other embodiments, the solution contains buffers or salts to promote annealing, and/or stabilization of the duplex strands.

In some embodiments, the dsRNA is transcribed from the vectors as two separate stands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. In some embodiments, a DNA duplex provided at each end with a promoter sequence can directly generate RNAs of defined length, and which can join in pairs to form a dsRNA. See, e.g., U.S. Pat. No. 5,795,715, incorporated herein by reference. RNA duplex formation may be initiated either inside or outside the cell.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 15, 19, 21, 25, 50, 100, 200, 300 or 400 bases up to the full length of the RNA molecule. As such there is no upper limit on the length of the dsRNA that can be used. For example, the dsRNA can range from about 21 base pairs (bp) of the gene to the full length of the gene or more.

Preferably, the dsRNAs to be used in this invention target at least one psyllid gene portion of at least 19 consecutive nucleotides occurring in identical sequence or with high sequence identity in the targeted psyllid (e.g., ACP or PoP). In one embodiment of this invention, such dsRNAs do not silence genes of a plant host, or of other non-target animals, such as ACP or PoP predators or animals such as other arthropods, spiders, reptiles, amphibians, birds, or mammals. Levels of homology between sequences of interest can be analyzed in available genome/transcriptome databases, e.g., by a BLAST search (see also www.ncbi.nlm.nih.gov/BLAST) or by hybridization with existing DNA libraries of representative non-target organisms. In one embodiment of this invention, the dsRNA or siRNA of the invention corresponds to an exon in a target gene.

As used herein, nucleotide sequences of RNA molecules can be identified by reference to DNA nucleotide sequences of the sequence listing. However, the person skilled in the art will understand whether RNA or DNA is meant depending on the context. Furthermore, the nucleotide sequence is identical between the types of polynucleotides except that the T-base is replaced by uracil (U) in RNA molecules.

It will be appreciated that the longer the total length of the first (sense) nucleotide sequence in the dsRNA of the invention is, the less stringent the requirements for sequence identity between the total sense nucleotide sequence and the corresponding sequence in the target gene becomes. The total first nucleotide sequence can have a sequence identity of at least about 75% with the corresponding target sequence, but higher sequence identity can also be used such as at least about 80%, at least about 85%, at least about 90%, at least about 95%, at least about 99% or about 100%. The first nucleotide sequence can also be identical to the corresponding part of the target gene. However, it is advised that the first nucleotide sequence includes a sequence of 19 or 20, or about 19 or about 20 consecutive nucleotides, or even of about 50 consecutive nucleotides, or about consecutive 100 nucleotides, or about 150 consecutive nucleotides with only one mismatch, preferably with 100% sequence identity, to the corresponding part of the target gene. For calculating the sequence identity and designing the corresponding first nucleotide sequence, the number of gaps should be minimized, particularly for the shorter sense sequences.

The length of the second (antisense) nucleotide sequence in the dsRNA of the invention is largely determined by the length of the first (sense) nucleotide sequence, and may correspond to the length of the latter sequence. However, it is possible to use an antisense sequence which differs in length by about 10% without any difficulties. Similarly, the nucleotide sequence of the antisense region is largely determined by the nucleotide sequence of the sense region, and may be identical to the complement of the nucleotide sequence of the sense region. Particularly with longer antisense regions, it is however possible to use antisense sequences with lower sequence identity to the complement of the sense nucleotide sequence, such as at least about 75% sequence identity, or least about 80%, or at least about 85%, more particularly with at least about 90% sequence identity, or at least about 95% sequence to the complement of the sense nucleotide sequence. Nevertheless, it is advised that the antisense nucleotide sequence always includes a sequence of 19 or 20, about 19 or about 20 consecutive nucleotides, although longer stretches of consecutive nucleotides such as about 50 nucleotide, or about 100 nucleotides, or about 150 nucleotides with no more than one mismatch, preferably with 100% sequence identity, to the complement of a corresponding part of the sense nucleotide sequence can also be used. Again, the number of gaps should be minimized, particularly for the shorter (19 to 50 nucleotides) antisense sequences.

In one embodiment of the invention, the DNA molecules according to the invention can comprise a DNA region encoding a spacer between the DNA region encoding the first and second nucleotide sequences. As indicated in WO 99/53050 the spacer may contain an intron to enhance gene silencing. A particularly preferred intron functional in cells of plants is the pdk intron (Flaveria trinervia pyruvate orthophosphate dikinase intron 2; see WO99/53050), the delta 12 desaturase intron from Arabidopsis (Smith et al., Nature, (2000) 407:319-20) or the intron of the rolA gene (Magrelli et al., Science (1994) 266:1986-1988; Spena and Langenkemper, Genet Res, (1997) 69:11-15).

In one embodiment of the invention, a dsRNA molecule may further comprise one or more regions having at least 94%, at least 95%, at least 96%, at least 97%, at least 98%, or at least 99%, sequence identity to regions of at least 19 consecutive nucleotides from the sense nucleotide sequence of the target gene, different from the at least 19 consecutive nucleotides as defined in the first region, and one or more regions having at least 94%, at least 95%, at least 96%, at least 97%, at least 98%, or at least 99%, sequence identity to at least 19 consecutive nucleotides from the complement of the sense nucleotide sequence of the target gene, different from the at least 19 consecutive nucleotides as defined in the second region, wherein these additional regions can base-pair amongst themselves.

Preferred sequences are listed in Table 1. The sequences are provided in the accompanying SEQ ID listing. SEQ ID NOs: 1-49 are the full length DNA sequences for selected PoP genes identified as described above. SEQ ID NOs:50-56 are the full length DNA sequences for selected ACP genes identified as described above. SEQ ID NOs:57-105 are preferred PoP sequences for use in dsRNA expression cassettes. SEQ ID NOs:106-113 are preferred ACP sequences for use in dsRNA expression cassettes.

TABLE 1

| | | | | Selected |
|---|---|---|---|---|
| | | | Full length | dsRNA |
| Organism | Code | Gene | Sequence | region |
| PoP | BcAN_14913 | Wnt-1 | SEQ ID | SEQ ID |
| | | | NO: 1 | NO: 57 |
| | | | | SEQ ID |
| | | | | NO: 58 |
| PoP | BcAN_18498 | Pangolin | SEQ ID | SEQ ID |
| | | | NO: 2 | NO: 59 |
| PoP | BcAN_00838 | Armadillo | SEQ ID | SEQ ID |
| | | | NO: 3 | NO: 60 |
| PoP | BcAN_23675 | Snf7 | SEQ ID | SEQ ID |
| | | | NO: 4 | NO: 61 |
| PoP | BcAN_05948 | Vps-4 | SEQ ID | SEQ ID |
| | | | NO: 5 | NO: 62 |
| PoP | BcAN_03041 | Vps-20 | SEQ ID | SEQ ID |
| | | | NO: 6 | NO: 63 |
| PoP | BcAN_00119 | v-ATPase A | SEQ ID | SEQ ID |
| | | | NO: 7 | NO: 64 |
| PoP | BcAN_02379 | v-ATPase B | SEQ ID | SEQ ID |
| | | | NO: 8 | NO: 65 |
| PoP | BcAN_10854 | v-ATPase D | SEQ ID | SEQ ID |
| | | | NO: 9 | NO: 66 |
| PoP | BcAN_02354 | v-ATPase E | SEQ ID | SEQ ID |
| | | | NO: 10 | NO: 67 |
| PoP | BcAN_10025 | ATG6 | SEQ ID | SEQ ID |
| | | | NO: 11 | NO: 68 |
| PoP | BcAN_03357 | dsRNAse1 | SEQ ID | SEQ ID |
| | | | NO: 12 | NO: 69 |
| PoP | BcAN_05172 | dsRNAse2 | SEQ ID | SEQ ID |
| | | | NO: 13 | NO: 70 |
| PoP | BcAN_15309 | dsRNAse3 | SEQ ID | SEQ ID |
| | | | NO: 14 | NO: 71 |
| PoP | BcGS_00182 | Trehalase | SEQ ID | SEQ ID |
| | | | NO: 15 | NO: 72 |
| PoP | BcGS_01180 | Maltase | SEQ ID | SEQ ID |
| | | | NO: 16 | NO: 73 |
| | BcGS_02877 | Heat Shock Protein 70 | SEQ ID | SEQ ID |
| | | (Hsp70) | NO: 17 | NO: 74 |
| PoP | BcGS_05866 | Trehalose | SEQ ID | SEQ ID |
| | | transporter 1 | NO: 18 | NO: 75 |
| | | (Tret1) | | |
| PoP | BcAN_19429 | Nicotinic | SEQ ID | SEQ ID |
| | | acetylcholine | NO: 19 | NO: 76 |
| | | receptor | | |
| PoP | BcGS_33684 | Aquaporin (AQP) | SEQ ID | SEQ ID |
| | | | NO: 20 | NO: 77 |
| PoP | BcAN_11253 | AP-1 | SEQ ID | SEQ ID |
| | | | NO: 21 | NO: 78 |
| PoP | BCAN_12335 | Clathrin heavy | SEQ ID | SEQ ID |
| | | chain | NO: 22 | NO: 79 |
| PoP | BcGS_03275 | Clathrin light | SEQ ID | SEQ ID |
| | | chain | NO: 23 | NO: 80 |
| PoP | BcAN_01232 | Rab GDP | SEQ ID | SEQ ID |
| | | | NO: 24 | NO: 81 |
| PoP | BcAN_01844 | Vesicle- | SEQ ID | SEQ ID |
| | | associated | NO: 25 | NO: 82 |
| | | membrane protein | | |
| | | 2/ | | |
| | | synaptobrevin- | | |
| | | binding protein | | |
| PoP | BcGS_02956 | Synaptotagmin-1 | SEQ ID | SEQ ID |
| | | | NO: 26 | NO: 83 |
| PoP | BcAN_10074 | Synaptotagmin- | SEQ ID | SEQ ID |
| | | 11 | NO: 27 | NO: 84 |
| PoP | BcAN_01478 | Muscle Actin | SEQ ID | SEQ ID |
| | | | NO: 28 | NO: 85 |
| PoP | BcAN_09262 | Actin- | SEQ ID | SEQ ID |
| | | interacting | NO: 29 | NO: 86 |
| | | protein 1 | | |
| PoP | BcGS_22823 | Actin-related | SEQ ID | SEQ ID |
| | | protein 3 | NO: 30 | NO: 87 |
| PoP | BcGS_10813 | BcGS 10813 | SEQ ID | NA |
| | | Probable actin- | NO: 31 | |
| | | related protein | | |
| | | 2/3 complex | | |
| | | subunit 3 | | |
| PoP | BcGS_01438 | Probable actin- | SEQ ID | SEQ ID |
| | | related protein | NO: 32 | NO: 88 |
| | | 2/3 complex | | |
| | | subunit 2 | | |
| PoP | BcAN_00891 | Beta-arrestin | SEQ ID | SEQ ID |
| | | | NO: 33 | NO: 89 |
| PoP | NA | Colifin/actin/ | SEQ ID | SEQ ID |
| | | depolymerization | NO: 34 | NO: 90 |
| | | factor | | |
| PoP | BcGS_00195 | Gelsolin | SEQ ID | SEQ ID |
| | | | NO: 35 | NO: 91 |
| PoP | BcGS_08234 | Ras-like GTP- | SEQ ID | SEQ ID |
| | | binding protein | NO: 36 | NO: 92 |
| | | Rho1 | | |
| PoP | BcGS_01214 | Cdc42 homolog | SEQ ID | SEQ ID |
| | | | NO: 37 | NO: 93 |
| PoP | BcAN_12177 | Vinculin | SEQ ID | SEQ ID |
| | | | NO: 38 | NO: 94 |
| PoP | BcGS_17389 | Wiskott-Aldrich | SEQ ID | SEQ ID |
| | | syndrome protein | NO: 39 | NO: 95 |
| | | (WASP) | | |
| PoP | BcGS_06785 | Wiskott-Aldrich | SEQ ID | SEQ ID |
| | | syndrome protein | NO: 40 | NO: 96 |
| | | (WASP) member 3 | | |
| PoP | BcGS_05363 | Cortactin | SEQ ID | SEQ ID |
| | | | NO: 41 | NO: 97 |
| PoP | BcGS_00175 | Delta-24 sterol | SEQ ID | SEQ ID |
| | | reductase | NO: 42 | NO: 98 |
| PoP | | C-7 cholesterol | SEQ ID | SEQ ID |
| | | desaturase From | NO: 43 | NO: 99 |
| | | PoP Genome | | |
| PoP | BcGS_04315 | Focal Adhesion | SEQ ID | SEQ ID |
| | | Kinase 1 | NO: 44 | NO: 100 |
| PoP | BcAN_01048 | RAC | SEQ ID | SEQ ID |
| | | | NO: 45 | NO: 101 |
| PoP | BcAN_01865 | GTPase H Ras | SEQ ID | SEQ ID |
| | | | NO: 46 | NO: 102 |
| PoP | BcAN_09831 | Actin-related | SEQ ID | SEQ ID |
| | | protein 2 | NO: 47 | NO: 103 |
| | | (Arp2/3) | | |
| PoP | BcAN_01762 | Crc, | SEQ ID | SEQ ID |
| | | Cryptocephal | NO: 48 | NO: 104 |
| PoP | BcAN_12524 | CLIPB-serine | SEQ ID | SEQ ID |
| | | protease 5-RA | NO: 49 | NO: 105 |
| ACP | Dcitr06g | ACP v-ATPase-A | SEQ ID | SEQ ID |
| | 09110.1.1 | | NO: 50 | NO: 106 |
| ACP | Dcitr09g | ACP v-ATPase-B | SEQ ID | SEQ ID |
| | 08730.1.1 | | NO: 51 | NO: 107 |
| ACP | Dcitr09g | ACP v-ATPase D | SEQ ID | SEQ ID |
| | 02100.1.1 | | NO: 52 | NO: 108 |
| ACP | Dcitr09g | ACP v-ATPase E | SEQ ID | SEQ ID |
| | 02500.1.1 | | NO: 53 | NO: 109 |
| | | | | SEQ ID |
| | | | | NO: 110 |
| ACP | Dcitr04g | Delta-24 sterol | SEQ ID | SEQ ID |
| | 03460.1.1 | reductase | NO: 54 | NO: 111 |
| ACP | DC3Osc03 | ACP C-7 | SEQ ID | SEQ ID |
| | 29905893- | cholesterol | NO: 55 | NO: 112 |
| | 29905495 | desaturase | | |
| ACP | Dcitr12g | ACP Crc | SEQ ID | SEQ ID |
| | 05570.1.1 | | NO: 56 | NO: 113 |

In particular embodiments, a dsRNA molecule of the present invention comprises a first (sense) strand that is 80%-100% identical to an RNA sequence encoded by any SEQ ID NOs. 1-113. For example, a dsRNA molecule that has 90%, 91%, 92%, 93%, 94%, 95%, 96%, 97%, 98%, 99%, 100% sequence identity to an RNA sequence encoded by any of SEQ ID NOs. 1-113. One of skill in the art will recognize that these whole number percentages encompass any portion or fraction of a percentage between 80% and 100%.

In some embodiments, the dsRNA constructs of the present invention comprise a first exogenous nucleic acid sequence having a sense sequence linked to its complementary antisense sequence and encoding a double stranded RNA (e.g., any of SEQ ID NOs:1-113 or portions thereof) that inhibits expression of a target RNA molecule. In some embodiments, the exogenous nucleic acid sequence is operably linked to a promoter suitable for use in a desired host cell such a plant cell, bacteria, or yeast. In some embodiments, the constructs comprise sense and antisense sequence corresponding to a portion of the target pest sequence joined by a linker so that a dsRNA is formed upon expression of the construct

In other embodiments, the RNA construct of the present invention comprises a sequence encoding an RNA molecule (e.g., any of SEQ ID NOs:1-113 or portions thereof) that inhibits expression of a target RNA molecule and that is flanked by convergent promoters. Suitable convergent promoter systems are described, for example, in He et al., J. Exper. Botany (2020) 71(9):2670-2677; Wu et al., Front. Plant Sci. (2017) 21 (doi.org/10.3389/fpls.2017.01454); and Zhang et al., Science (2015) 347(6225):991-994.

### II. Transgenic Plants

In some embodiments, the present invention provides transgenic plants that express the dsRNA constructs and systems described above. It is contemplated that pests (e.g., psyllids) feeding on the transgenic plants ingest the dsRNA molecules, which in turn decrease the abundance of target RNA within the pest species. This results in decreased pest infestation and decreased plant damage. In some particularly preferred embodiments, the result is a decrease or reduction in citrus greening disease.

A heterologous gene encoding an dsRNA of the present invention, which includes variants, includes any suitable sequence that encodes a double stranded molecule specific for a pest target RNA molecule. Preferably, the heterologous gene is provided within an expression vector such that transformation with the vector results in expression of the double stranded RNA molecule; suitable vectors are described below.

In yet other embodiments of the present invention, a transgenic plant comprises a heterologous gene encoding a dsRNA of the present invention operably linked to an inducible promoter (or convergent promoters as appropriate), and is grown either in the presence of the an inducing agent, or is grown and then exposed to an inducing agent. In still other embodiments of the present invention, a transgenic plant comprises a heterologous gene encoding an dsRNA of the present invention and/or a sense or antisense sequence operably linked to a promoter (or convergent promoters as appropriate), which is either tissue specific or developmentally specific, and is grown to the point at which the tissue is developed or the developmental stage at which the developmentally-specific promoter is activated. Such promoters include seed and root specific promoters. In still other embodiments of the present invention, the transgenic plant comprises a dsRNA of the present invention and/or a sense or antisense sequence operably linked to constitutive promoter (or convergent promoters as appropriate). In further embodiments, the transgenic plants of the present invention express at least one ds RNA molecule at a level sufficient to reduce the proliferation of psyllids as compared to the proliferation of psyllids (e.g., PoP or ACP) observed in a nontransgenic plant.

1. Plants

The methods of the present invention are not limited to any particular plant. Indeed, a variety of plants are contemplated, including but not limited to potatoes, tomatoes, and citrus plants such as lime, lemon, orange and grapefruit trees, can be transformed with heterologous genes, including commercial cultivars. In cases where that is not possible, non-commercial cultivars of plants can be transformed, and the trait for expression of the dsRNA of the present invention moved to commercial cultivars by breeding techniques well-known in the art.

2. Vectors

The methods of the present invention contemplate the use of at least one heterologous gene encoding a dsRNA. Heterologous genes intended for expression in plants are first assembled in expression cassettes comprising a promoter. Methods which are well known to those skilled in the art may be used to construct expression vectors containing a heterologous gene and appropriate transcriptional and translational control elements. These methods include in vitro recombinant DNA techniques, synthetic techniques, and in vivo genetic recombination. Such techniques are widely described in the art (See e.g., Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y).

In general, these vectors comprise a nucleic acid sequence of the invention encoding a ds RNA operably linked to a promoter (or convergent promoters as appropriate) and other regulatory sequences (e.g., enhancers, polyadenylation signals, etc.) required for expression in a plant.

Promoters include but are not limited to constitutive promoters, tissue-, organ- and developmentally-specific promoters, and inducible promoters. Examples of promoters include but are not limited to: constitutive promoter 35S of cauliflower mosaic virus; a wound-inducible promoter from tomato, leucine amino peptidase (“LAP,” Chao et al. (1999) Plant Physiol 120: 979-992); a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (U.S. Pat. No. 5,187,267); a tetracycline-inducible promoter (U.S. Pat. No. 5,057,422); and seed-specific promoters, such as those for seed storage proteins (e.g., phaseolin, napin, oleosin, and a promoter for soybean beta conglycin (Beachy et al. (1985) EMBO J. 4: 3047-3053)). In some preferred embodiments, the promoter is a phaseolin promoter. All references cited herein are incorporated in their entirety.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its correct polyadenylation. Appropriate transcriptional terminators and those which are known to function in plants include, but are not limited to, the CaMV 35S terminator, the tml terminator, the pea rbcS E9 terminator, and the nopaline and octopine synthase terminator (See e.g., Odell et al. (1985) Nature 313:810; Rosenberg et al. (1987) Gene, 56:125; Guerineau et al. (1991) Mol. Gen. Genet., 262:141; Proudfoot (1991) Cell, 64:671; Sanfacon et al. Genes Dev., 5:141; Mogen et al. (1990) Plant Cell, 2:1261; Munroe et al. (1990) Gene, 91:151; Ballad et al. (1989) Nucleic Acids Res. 17:7891; Joshi et al. (1987) Nucleic Acid Res., 15:9627).

In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize Adhl gene have been found to significantly enhance the expression of the wild-type gene under its cognate promoter when introduced into maize cells (Calais et al. (1987) Genes Develop. 1: 1183). Intron sequences have been routinely incorporated into plant transformation vectors, typically within the non-translated leader.

In some embodiments of the present invention, the construct for expression of the nucleic acid sequence of interest also includes a regulator such as a nuclear localization signal (Calderone et al. (1984) Cell 39:499; Lassoer et al. (1991) Plant Molecular Biology 17:229), a plant translational consensus sequence (Joshi (1987) Nucleic Acids Research 15:6643), an intron (Luehrsen and Walbot (1991) Mol. Gen. Genet. 225:81), and the like, operably linked to the nucleic acid sequence encoding the RNAi gene and/or an antisense or sense sequence.

In preparing a construct comprising a nucleic acid sequence encoding a dsRNA of the present invention, various DNA fragments can be manipulated, so as to provide for the DNA sequences in the desired orientation (e.g., sense or antisense) orientation. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like is preferably employed, where insertions, deletions or substitutions (e.g., transitions and transversions) are involved.

Numerous transformation vectors including but not limited to plasmid and viral vectors are available for plant transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics (Messing and Vierra (1982) Gene 19: 259; Bevan et al. (1983) Nature 304:184), the bar gene which confers resistance to the herbicide phosphinothricin (White et al. (1990) Nucl Acids Res. 18:1062; Spencer et al. (1990) Theor. Appl. Genet. 79:625), the hph gene which confers resistance to the antibiotic hygromycin (Blochlinger and Diggelmann (1984) Mol. Cell. Biol. 4:2929), and the dhfr gene, which confers resistance to methotrexate (Bourouis et al. (1983) EMBO J., 2:1099).

In some preferred embodiments, the vector is adapted for use in an Agrobacterium mediated transfection process (See e.g., U.S. Pat. Nos. 5,981,839; 6,051,757; 5,981,840; and 4,940,838; all of which are incorporated herein by reference). Construction of recombinant Ti and Ri plasmids in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The second system is called the “binary” system in which two plasmids are used; the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PAL4404. Some of these vectors are commercially available.

In other embodiments of the invention, the nucleic acid sequence of interest is targeted to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (U.S. Pat. No. 5,501,967). One of skill in the art knows that homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

In some embodiments of the present invention the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278).

3. Transformation Techniques

Once a nucleic acid sequence encoding a dsRNA of the present invention is operatively linked to an appropriate promoter(s) and inserted into a suitable vector for the particular transformation technique utilized (e.g., one of the vectors described above), the recombinant DNA described above can be introduced into the plant cell in a number of art-recognized ways. Those skilled in the art will appreciate that the choice of method might depend on the type of plant targeted for transformation. In some embodiments, the vector is maintained episomally. In other embodiments, the vector is integrated into the genome.

In some embodiments, the vector is introduced through ballistic particle acceleration using devices (e.g., available from Agracetus, Inc., Madison, Wis. and Dupont, Inc., Wilmington, Del). (See e.g., U.S. Pat. No. 4,945,050; and McCabe et al. (1988) Biotechnology 6:923). See also, Weissinger et al. (1988) Annual Rev. Genet. 22:421; Sanford et al. (1987) Particulate Science and Technology, 5:27 (onion); Svab et al. (1990) Proc. Natl. Acad. Sci. USA, 87:8526 (tobacco chloroplast); Christou et al. (1988) Plant Physiol., 87:671 (soybean); McCabe et al. (1988) Bio/Technology 6:923 (soybean); Klein et al. (1988) Proc. Natl. Acad. Sci. USA, 85:4305 (maize); Klein et al. (1988) Bio/Technology, 6:559 (maize); Klein et al. (1988) Plant Physiol., 91:4404 (maize); Fromm et al. (1990) Bio/Technology, 8:833; and Gordon-Kamm et al. (1990) Plant Cell, 2:603 (maize); Koziel et al. (1993) Biotechnology, 11:194 (maize); Hill et al. (1995) Euphytica, 85:119 and Koziel et al. (1996) Annals of the New York Academy of Sciences 792:164; Shimamoto et al. (1989) Nature 338: 274 (rice); Christou et al. (1991) Biotechnology, 9:957 (rice); Dana et al. (1990) Bio/Technology 8:736 (rice); European Patent Application EP 0 332 581 (orchardgrass and other Pooideae); Vasil et al. (1993) Biotechnology, 11: 1553 (wheat); Weeks et al. (1993) Plant Physiol., 102: 1077 (wheat); Wan et al. (1994) Plant Physiol. 104: 37 (barley); Jahne et al. (1994) Theor. Appl. Genet. 89:525 (barley); Knudsen and Muller (1991) Planta, 185:330 (barley); Umbeck et al. (1987) Bio/Technology 5: 263 (cotton); Casas et al. (1993) Proc. Natl. Acad. Sci. USA 90:11212 (sorghum); Somers et al. (1992) Bio/Technology 10:1589 (oat); Torbert et al. (1995) Plant Cell Reports, 14:635 (oat); Weeks et al. (1993) Plant Physiol., 102:1077 (wheat); Chang et al., WO 94/13822 (wheat) and Nehra et al. (1994) The Plant Journal, 5:285 (wheat).

In other embodiments, direct transformation in the plastid genome is used to introduce the vector into the plant cell (See e.g., U.S. Pat. Nos. 5,451,513; 5,545,817; 5,545,818; PCT application WO 95/16783). The basic technique for chloroplast transformation involves introducing regions of cloned plastid DNA flanking a selectable marker together with the nucleic acid encoding the RNA sequences of interest into a suitable target tissue (e.g., using biolistics or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al. (1990) PNAS, 87:8526; Staub and Maliga, (1992) Plant Cell, 4:39). The presence of cloning sites between these markers allowed creation of a plastid targeting vector introduction of foreign DNA molecules (Staub and Maliga (1993) EMBO J., 12:601). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga (1993) PNAS, 90:913). Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of the RNAs encoded by the DNA molecule.

In other embodiments, vectors useful in the practice of the present invention are microinjected directly into plant cells by use of micropipettes to mechanically transfer the recombinant DNA (Crossway (1985) Mol. Gen. Genet, 202:179). In still other embodiments, the vector is transferred into the plant cell by using polyethylene glycol (Krens et al. (1982) Nature, 296:72; Crossway et al. (1986) BioTechniques, 4:320); fusion of protoplasts with other entities, either minicells, cells, lysosomes or other fusible lipid-surfaced bodies (Fraley et al. (1982) Proc. Natl. Acad. Sci., USA, 79:1859); protoplast transformation (EP 0 292 435); direct gene transfer (Paszkowski et al. (1984) EMBO J., 3:2717; Hayashimoto et al. (1990) Plant Physiol. 93:857).

In still further embodiments, the vector may also be introduced into the plant cells by electroporation (Fromm, et al. (1985) Proc. Natl Acad. Sci. USA 82:5824; Riggs et al. (1986) Proc. Natl. Acad. Sci. USA 83:5602). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In addition to direct transformation, in some embodiments, the vectors comprising a nucleic acid sequence encoding a dsRNA of the present invention are transferred using Agrobacterium-mediated transformation (Hinchee et al. (1988) Biotechnology, 6:915; Ishida et al. (1996) Nature Biotechnology 14:745). Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Heterologous genetic sequences (e.g., nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome (Schell (1987) Science, 237: 1176). Species which are susceptible infection by Agrobacterium may be transformed in vitro. Alternatively, plants may be transformed in vivo, such as by transformation of a whole plant by Agrobacteria infiltration of adult plants, as in a “floral dip” method (Bechtold N, Ellis J, Pelletier G (1993) Cr. Acad. Sci. III—Vie 316: 1194-1199). In other preferred embodiments, transformation via Rhizobium rhizogenes is utilized. See, e.g., U.S. Ser. No. 15/353,645 and Irigoyene al. Nature Comm. (2020) 11:5802, each incorporated herein by reference in its entirety.

In still other embodiments, virus-vector transfection delivery of dsRNA is utilized. In some preferred embodiments, the dsRNA is delivered with a Citrus tristeza virus vector. See e.g., Hajeri et al., J. Biotech. (2014) 42-49. In other preferred embodiments, the dsRNA is delivered via an Independent-mobile RNA (iRNA) expression vector.

4. Regeneration

After selecting for transformed plant material that can express the heterologous gene encoding a dsRNA of the present invention, whole plants are regenerated. Plant regeneration from cultured protoplasts is described in Evans et al. (1983) Handbook of Plant Cell Cultures, Vol. 1: (MacMillan Publishing Co. New York); and Vasil I. R. (ed.), Cell Culture and Somatic Cell Genetics of Plants, Acad. Press, Orlando, Vol. I (1984), and Vol. III (1986). It is known that many plants can be regenerated from cultured cells or tissues, including but not limited to all major species of sugarcane, sugar beet, cotton, fruit and other trees, legumes and vegetables, and monocots (e.g., the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos germinate and form mature plants. The culture media will generally contain various amino acids and hormones, such as auxin and cytokinins. Shoots and roots normally develop simultaneously. Efficient regeneration will depend on the medium, on the genotype, and on the history of the culture. The reproducibility of regeneration depends on the control of these variables.

5. Generation of Trans Genic Lines

Transgenic lines are established from transgenic plants by tissue culture propagation. The presence of nucleic acid sequences encoding a dsRNA of the present invention (including mutants or variants thereof) may be transferred to related varieties by traditional plant breeding techniques.

6. Other Delivery Methods

Although plant delivery of a dsRNA is an embodiment of this invention, in accordance with this invention, application of the dsRNA of the invention can be done in several ways, and need not be by way of a plant expressing a dsRNA. Any method of delivery of dsRNA not contained in a plant cell is included herein, e.g., in vitro or in vivo produced dsRNA applied to an insect diet or feed, or microbially- or yeast-expressed dsRNA. The dsRNA can be applied (e.g., typically applied) on plants on which target psyllids feed by spraying or injecting or by passive uptake following cuticle disruption a solution of eg., microbial organisms, yeast spores, cells, or cells or via composistion comprising the dsRNA molecules, for example dsRNA molecules suspended in inert molecules such as nanoparticles or claynanosheets comprising the dsRNA of the invention. The dsRNA species of the present invention can be applied on plants by spraying a culture, culture extract, culture supernatant, or a combination thereof. In some preferred embodiments, the sprayed material comprises a microbe-expressed dsRNA or a suspension of dsRNA or other dsRNA composition as described above. Thus, the present invention includes microbes comprising genetic elements allowing for the expression of any of the dsRNA species described herein as well as delivery compositions comprising the dsRNA molecules in a suitable carrier alone or in combination with molecule ssuch as nanoparticles,

In particular embodiments, the present invention provides a composition having an inhibitory nucleic acid specific for an mRNA or fragment thereof represented by one or more of SEQ ID NOs. 1-113 or a fragment or homologue thereof. Typically, dsRNAs of the present invention are provided to a target insect pest in an amount sufficient to inhibit production of the polypeptide encoded by one or more of the full-length genes targeted by SEQ ID NOs. 1-113 or homologues and alleles thereof. For example when a target psyllid is feeding on a plant or cell expressing, or containing, or coated with an inhibitory nucleic acid, the insect ingests a sufficient level of dsRNA of SEQ ID NOs. 1-113 or a portion thereof to result in a phenotypic effect. In particular embodiments, a combination of two or more dsRNAs of SEQ ID NOs. 1-113 are combined in a single insecticidal composition, for example a combination of dsRNA comprising SEQ ID NO. 106 and SEQ ID NO. 111. In addition to an inhibitory nucleic acid, an insecticidal composition of the present invention can contain one or more phagostimulants, pesticides, fungicides, or combinations thereof. The composition can be formulated to be coated to be coated on a plant, plant part, or seed. In certain aspects the inhibitory nucleic acid is combined with one or more excipients, buffering agents, carriers, etc. excipients, buffering agents, and carriers are well known in the art.

Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulo se phthalate, noncrystalline cellulose, magnesium aluminum silicate, triethanolamine, polyvinyl alcohol, polyvinylpyrrolidone, sugars and starches.

The coating can be formulated as a spray or dip so that the inhibitory nucleic acids remain on the plant material and remain able to inhibit target protein expression in a target psyllid as the plant matures and develops. For example, the seed of a plant can be coated with a composition comprising an amount of one or more of the disclosed inhibitory nucleic acids effective to inhibit or reduce psyllid infection or citrus greening disease in the plant in combination with an excipient.

### REFERENCES

Brown, J. K., Rehman, M., Rogan, D., Martin, R. R., and Idris, A. M. 2010. First report of “Candidatus Liberibacter psyllaurous” (syn “Ca. L. solanacearum”) associated with the ‘tomato vein-greening’ and ‘tomato psyllid yellows’ diseases in commercial greenhouses in Arizona. Plant Dis. 94:376.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, plant biology, biochemistry, or related fields are intended to be within the scope of the following claims.

1. A double-stranded ribonucleic acid (dsRNA) comprising a sense region with at least 80% sequence identity to a sequence comprising at least 15 consecutive nucleotides up to the entire length of an RNA sequence encoded by a sequence selected from the group consisting of SEQ ID NOs: 1-113, and an antisense region comprising a second sequence complementary entirely to the sense region.

2. The double stranded nucleic acid sequence of claim 1, wherein the dsRNA comprises a sense region with at least 90% sequence identity to a sequence comprising at least consecutive nucleotides up to the entire length of an RNA sequence encoded by a sequence selected from the group consisting of SEQ ID NOs: 1-113, and an antisense region comprising a second sequence complementary entirely to the sense region.

3. The double stranded nucleic acid sequence of claim 1, wherein the dsRNA comprises a sense region with at least 95% sequence identity to a sequence comprising at least consecutive nucleotides up to the entire length of an RNA sequence encoded by a sequence selected from the group consisting of SEQ ID NOs: 1-113, and an antisense region comprising a second sequence complementary entirely to the sense region.

4. The double stranded nucleic acid sequence of claim 1, wherein the dsRNA comprises a sense region with at least 99% sequence identity to a sequence comprising at least consecutive nucleotides up to the entire length of an RNA sequence encoded by a sequence selected from the group consisting of SEQ ID NOs: 1-113, and an antisense region comprising a second sequence complementary entirely to the sense region.

5. The double stranded nucleic acid sequence of claim 1, wherein the dsRNA comprises a sense region with 100% sequence identity to a sequence comprising at least 15 consecutive nucleotides up to the entire length of an RNA sequence encoded by a sequence selected from the group consisting of SEQ ID NOs: 1-113, and an antisense region comprising a second sequence complementary entirely to the sense region.

6. The double stranded nucleic acid sequence of claim 1, wherein the dsRNA sequence is encoded by a selected from the group consisting of SEQ ID NOs:1 to 49.

7. The double stranded nucleic acid sequence of claim 1, wherein the dsRNA sequence is encoded by a selected from the group consisting of SEQ ID NOs:50 to 56.

8. The double stranded nucleic acid sequence of claim 1, wherein the dsRNA sequence is encoded by a selected from the group consisting of SEQ ID NOs:57 to 113.

9. The double stranded nucleic acid sequence of claim 1, wherein the dsRNA sequence is encoded by a selected from the group consisting of SEQ ID NOs:57 to 105.

10. The double stranded nucleic acid sequence of claim 1, wherein the dsRNA sequence is encoded by a selected from the group consisting of SEQ ID NOs:106 to 113.

11. The double stranded nucleic acid sequence of claim 1, wherein the sense region comprises at least 21 consecutive nucleotides up to the entire length of a sequence selected from the group consisting of SEQ ID NOs: 1-113.

12-13. (canceled)

14. A transgenic plant, transgenic plant cell, or transgenic seed comprising the dsRNA sequence of claim 1.

15. (canceled)

16. A DNA molecule comprising a promoter functional in a host cell and a DNA encoding a dsRNA comprising a first region and a second region, wherein the first region comprises a sense region with at least 80% sequence identity to a sequence comprising at least consecutive nucleotides up to the entire length of a sequence selected from the group consisting of SEQ ID NOs: 1-113, and a second region complementary entirely to the sense region.

17-21. (canceled)

22. A DNA molecule comprising convergent promoters functional in a host cell flanking a DNA segment with at least 80% sequence identity to a sequence comprising at least consecutive nucleotides up to the entire length of a sequence selected from the group consisting of SEQ ID NOs: 1-113.

23-28. (canceled)

29. A method of controlling psyllids comprising, planting or growing a transgenic plant expressing the dsRNA of claim 1 and allowing one or more psyllids to ingest an effective amount of the dsRNA, thereby controlling the one or more psyllids, and/or interfering with Ca. Liberibacter transmission that results in abatement by any mode of interference resulting from dsRNA activity.

30. The method of claim 29, wherein the psyllids are Bactericera cockerelli.

31. The method of claim 29, wherein the psyllids are Diaphorina citri.

32-39. (canceled)
